(19) United States
(12) Patent Application Publication
(10) Pub. No.: US 2022/0411829 A1
(43) Pub. Date: Dec. 29, 2022

(54) METHODS AND COMPOSITIONS FOR PRODUCING ETHYLENE FROM RECOMBINANT MICROORGANISMS

(71) Applicant: CEMVITA FACTORY, INC., Houston, TX (US)

(72) Inventors: Tahereh KARIMI, Houston, TX (US); Truong Huu NGUYEN, Houston, TX (US); Miguel Eugenio CUEVA, Houston, TX (US)

(21) Appl. No.: 17/756,400

(22) PCT Filed: Dec. 2, 2020
(86) PCT No.: PCT/US2020/062938

§ 371 Date: May 24, 2022

Related U.S. Application Data
(60) Provisional application No. 62/942,895, filed on Dec. 3, 2019.

(51) Int. Cl. C12P 5/02; C12N 1/20; C12N 15/52

(52) U.S. Cl. CPC C12P 5/026; C12N 1/20; C12N 15/52; C12Y 203/03001; C12Y 101/01041; C12Y 207/09002; C12N 2500/02

(57) ABSTRACT

The present disclosure relates to recombinant microorganisms having an improved ethylene producing ability, methods of producing the same, and methods of producing ethylene. A benefit of the recombinant microorganisms and the methods disclosed herein can include increased production of ethylene from microbial cultures. An additional benefit can be the use of carbon dioxide to produce bio-ethylene useful as a feedstock for the production of plastics, textiles, and chemical materials, and for use in other applications. Another benefit of the methods and systems disclosed herein can include reduction of excess carbon dioxide from the environment.

### CROSS-REFERENCE

This application claims the benefit of U.S. Provisional Application No. 62/942,895, filed Dec. 3, 2019 which is incorporated herein by reference.

### TECHNICAL FIELD

The present disclosure relates to recombinant microorganisms having an improved ethylene producing ability, methods of producing the same, and methods of harvesting ethylene from such recombinant organisms. A benefit of the recombinant microorganisms and the methods disclosed herein can include increased production of ethylene from microbial cultures. An additional benefit can be the use of carbon dioxide to produce bio-ethylene useful as a feedstock for the production of plastics, textiles, and chemical materials, and for use in other applications. Another benefit of the methods and systems disclosed herein can include reduction of excess carbon dioxide from the environment.

### BACKGROUND

The increased demand for power worldwide has led to an excess of carbon dioxide from burning fossil fuels such as oil and gas, contributing substantially to what many are calling a global warming crisis. Industry is so desperate to prevent carbon dioxide from entering the atmosphere that they have resorted to sequestering carbon dioxide from exhaust streams and the atmosphere. They then store the carbon dioxide in subterranean environments. However, all current known methods just remove carbon dioxide from the atmosphere by storing it under ground. They do not actually convert the carbon dioxide back into any other useful material.

The limited supply of petroleum and its harmful effects on the environment have prompted developments in renewable sources of fuels and chemicals. Ethylene is the most widely produced organic compound in the world, useful in a broad spectrum of industries including plastics, solvents, and textiles. Ethylene is currently produced by steam cracking fossil fuels or dehydrogenating ethane. With millions of metric tons of ethylene being produced each year, however, more than enough carbon dioxide is produced by such processes to greatly contribute to the global carbon footprint. Producing ethylene through renewable methods would accordingly help to meet the huge demand from the energy and chemical industries, while also helping to protect the environment.

Since ethylene is a potentially renewable feedstock, there has been a great deal of interest in developing technologies to produce ethylene from renewable sources, such as carbon dioxide and biomass. Bio-ethylene is currently produced using ethanol derived from corn or sugar cane. A variety of microbes, including bacteria and fungi, naturally produce ethylene in small amounts. Heterologous expression of an ethylene producing enzyme has been demonstrated in several microbial species, where the hosts have been able to utilize a variety of carbon sources, including lignocellulose and carbon dioxide.

Based on modern history, it is fair to say that excess carbon dioxide in the atmosphere will not be reduced until it becomes profitable to reduce it. There remains a need for improvements in microbial bio-ethylene systems and processes, in order to produce ethylene at a commercial scale. There remains a need to produce hydrocarbons through more efficient renewable technologies. There remains a need to remove excess carbon dioxide from the atmosphere. There remains a need for improved methods to produce ethylene from a renewable feedstock for industrial and commercial applications.

### SUMMARY

Embodiments herein are directed to a recombinant microorganism having an improved ethylene producing ability, wherein the recombinant microorganism expresses at least one ethylene forming enzyme (EFE) protein having an amino acid sequence at least 95% identical to SEQ ID NO: 1 (see attached Appendix) by expressing a non-native EFE expressing nucleotide sequence, wherein an amount of EFE protein produced by the recombinant microorganism is greater than that produced relative to a control microorganism lacking the non-native EFE expressing nucleotide sequence. In an embodiment, the recombinant microorganism also expresses at least one alpha-ketoglutarate permease (AKGP) protein having an amino acid sequence at least 95% identical to SEQ ID NO: 2 (see attached Appendix) by expressing a non-native AKGP expressing nucleotide sequence, wherein an amount of AKGP protein produced by the recombinant microorganism is greater than that produced relative to a control microorganism lacking the non-native AKGP expressing nucleotide sequence. In an embodiment, the amount of EFE protein produced by the recombinant microorganism is from about 5% to about 200% or more greater than that produced relative to the control microorganism lacking the non-native EFE expressing nucleotide sequence.

In various embodiments, the recombinant microorganism includes a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, or a plant cell.

In an embodiment, the non-native EFE expressing nucleotide sequence is inserted into a bacterial vector plasmid, a high copy number bacterial vector plasmid, a bacterial vector plasmid having an inducible promoter, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 3 (see attached Appendix), and the non-native EFE expressing nucleotide sequence is inserted into a vector plasmid of a Chlamydomonas sp. bacterium.

In an embodiment, a non-native EFE expressing nucleotide sequence and a non-native AKGP expressing nucleotide sequence are inserted into a bacterial vector plasmid, a high copy number bacterial vector plasmid, a bacterial vector plasmid having an inducible promoter, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 4 (see attached Appendix), and the non-native EFE expressing nucleotide sequence and the AKGP expressing nucleotide sequence are inserted into a vector plasmid of an Escherichia sp. bacterium.

In an embodiment, the recombinant microorganism further includes a non-native AKGP expressing nucleotide sequence, wherein the non-native EFE expressing nucleotide sequence and non-native AKGP expressing nucleotide sequence express a combined amino acid sequence at least 95% identical to SEQ ID NO. 5 (see attached Appendix), and the non-native EFE expressing nucleotide sequence and non-native AKGP expressing nucleotide sequence are inserted into a bacterial plasmid of a Synechococcus sp. bacterium. In another embodiment, the recombinant microorganism further includes a non-native AKGP expressing nucleotide sequence, wherein the non-native EFE expressing nucleotide sequence and non-native AKGP expressing nucleotide sequence express a combined amino acid sequence at least 95% identical to SEQ ID NO. 6 (see attached Appendix), and the non-native EFE expressing nucleotide sequence and non-native AKGP expressing nucleotide sequence are inserted into a bacterial plasmid of a Synechococcus sp. bacterium.

In certain embodiments, the recombinant microorganism expresses at least one phosphoenolpyruvate synthase (PEP) protein having an amino acid sequence at least 95% identical to SEQ ID NO. 15 by expressing a non-native PEP expressing nucleotide sequence, wherein an amount of PEP protein produced by the recombinant microorganism is greater than that produced relative to a control microorganism lacking the non-native PEP expressing nucleotide sequence, and wherein an amount of AKG produced by the recombinant microorganism is greater than that produced relative to the control microorganism. In certain such embodiments, the recombinant microorganism includes a microorganism selected from the group consisting of a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.

In certain embodiments, the recombinant microorganism expresses at least one phosphoenolpyruvate synthase (PEP) protein having an amino acid sequence at least 95% identical to SEQ ID NO. 15 by expressing a non-native PEP expressing nucleotide sequence, wherein an amount of PEP protein produced by the recombinant microorganism is greater than that produced relative to a control microorganism lacking the non-native PEP expressing nucleotide sequence, and wherein an amount of AKG produced by the recombinant microorganism is greater than that produced relative to the control microorganism.

In certain embodiments, the recombinant microorganism expresses at least one citrate synthase protein having an amino acid sequence at least 95% identical to SEQ ID NO. 17 by expressing a non-native citrate synthase expressing nucleotide sequence, wherein an amount of citrate synthase protein produced by the recombinant microorganism is greater than that produced relative to a control microorganism lacking the non-native citrate synthase expressing nucleotide sequence.

In certain embodiments, the recombinant microorganism expresses at least one isocitrate dehydrogenase (IDH) protein having an amino acid sequence at least 95% identical to SEQ ID NO. 20 by expressing a non-native IDH expressing nucleotide sequence, wherein an amount of IDH protein produced by the recombinant microorganism is greater than that produced relative to a control microorganism lacking the non-native IDH expressing nucleotide sequence, and wherein an amount of AKG produced by the recombinant microorganism is greater than that produced relative to the control microorganism.

In certain embodiments, the recombinant microorganism contains a deletion in a glucose-1-phosphate adenylyltransferase expressing nucleotide sequence, wherein an amount of glucose-1-phosphate adenylyltransferase protein produced by the recombinant microorganism is less than that produced relative to a control microorganism lacking the deletion.

In certain embodiments, the recombinant microorganism expresses at least one sucrose permease protein having an amino acid sequence at least 95% identical to SEQ ID NO. 24 by expressing a non-native sucrose permease expressing nucleotide sequence,

wherein an amount of sucrose permease protein produced by the recombinant microorganism is greater than that produced relative to a control microorganism lacking the non-native sucrose permease expressing nucleotide sequence.

In certain embodiments, the recombinant microorganism expresses at least one sucrose permease protein having an amino acid sequence at least 95% identical to SEQ ID NO. 24 by expressing a non-native sucrose permease expressing nucleotide sequence,

wherein an amount of sucrose permease protein produced by the recombinant microorganism is greater than that produced relative to a control microorganism lacking the non-native sucrose permease expressing nucleotide sequence.

In certain embodiments, the recombinant microorganism expresses at least one sucrose permease protein having an amino acid sequence at least 95% identical to SEQ ID NO. 24 by expressing a non-native sucrose permease expressing nucleotide sequence,

wherein an amount of sucrose permease protein produced by the recombinant microorganism is greater than that produced relative to a control microorganism lacking the non-native sucrose permease expressing nucleotide sequence.

In certain embodiments, the recombinant microorganism expresses at least one protein selected from the group consisting of a sucrose phosphate synthase protein having an amino acid sequence at least 95% identical to SEQ ID NO. 26, a sucrose-6-phosphatase protein having an amino acid sequence at least 95% identical to SEQ ID NO. 28, a glycogen phosphorylase protein having an amino acid sequence at least 95% identical to SEQ ID NO. 30, and a UTP-glucose-1-phosphate uridylyltransferase protein having an amino acid sequence at least 95% identical to SEQ ID NO. 32, by expressing a non-native nucleotide sequence encoding the at least one protein, wherein an amount of the at least one protein produced by the recombinant microorganism is greater than that produced relative to a control microorganism lacking the non-native nucleotide sequence encoding the at least one protein, wherein an amount of sucrose produced by the recombinant microorganism is greater than that produced relative to the control microorganism.

In certain embodiments, the recombinant microorganism contains at least one deletion in at least one nucleotide sequence, wherein the at least one nucleotide sequence encodes at least one protein selected from an invertase protein having an amino acid sequence at least 95% identical to SEQ ID NO. 34, a glucosylglycerol-phosphate synthase protein having an amino acid sequence at least 95% identical to SEQ ID NO. 36, and a glycogen synthase protein having an amino acid sequence at least 95% identical to SEQ ID NO. 38, wherein an amount of the at least one protein produced by the recombinant microorganism is less than that produced relative to a control microorganism lacking the at least one deletion.

Embodiments herein are directed to methods of producing a recombinant microorganism having an improved ethylene producing ability. In an embodiment, the method includes producing the recombinant microorganism by inserting a non-native EFE expressing nucleotide sequence or a combined non-native EFE expressing nucleotide sequence and non-native AKGP expressing nucleotide sequence into a bacterial plasmid of a microorganism, wherein the non-native EFE expressing nucleotide sequence has a nucleotide sequence at least 95% identical to SEQ ID NO. 3 or SEQ ID NO. 4. In another embodiment, the combined non-native EFE expressing nucleotide sequence and non-native AKGP expressing nucleotide sequence expresses an amino acid sequence at least 95% identical to SEQ ID NO. 5 or SEQ ID NO. 6. In another embodiment, the combined non-native EFE expressing nucleotide sequence and non-native AKGP expressing nucleotide sequence has a nucleotide sequence at least 95% identical to SEQ ID NO. 7 (See Appendix).

In various embodied methods, the microorganism includes a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, or a plant cell.

In an embodiment of methods herein, the non-native EFE expressing nucleotide sequence has a nucleotide sequence at least 95% identical to SEQ ID NO. 3 and the microorganism is a Chlamydomonas sp. bacterium. In another embodiment, the non-native EFE expressing nucleotide sequence has a nucleotide sequence at least 95% identical to SEQ ID NO. 4 and the microorganism is an Escherichia sp. bacterium. In another embodiment, the combined non-native EFE expressing nucleotide sequence and non-native AKGP expressing nucleotide sequence expresses an amino acid sequence at least 95% identical to SEQ ID NO. 5 or SEQ ID NO. 6, and the microorganism is a Synechococcus sp. bacterium. In another embodiment, the combined non-native EFE expressing nucleotide sequence and non-native AKGP expressing nucleotide sequence has a nucleotide sequence at least 95% identical to SEQ ID NO. 7 and the microorganism is Synechococcus sp. bacterium.

Methods of producing ethylene are embodied herein. An embodiment of such a method includes providing a recombinant microorganism having an improved ethylene producing ability, wherein the recombinant microorganism expresses at least one ethylene forming enzyme (EFE) protein having an amino acid sequence at least 95% identical to SEQ ID NO: 1 by expressing a non-native EFE expressing nucleotide sequence, wherein an amount of EFE protein produced by the recombinant microorganism is greater than that produced relative to a control microorganism lacking the non-native EFE expressing nucleotide sequence; culturing the recombinant microorganism in a bioreactor culture vessel under conditions sufficient to produce ethylene in the bioreactor culture vessel; and harvesting ethylene from the bioreactor culture vessel.

In an embodiment of methods of producing ethylene herein, the recombinant microorganism contains a non-native EFE expressing nucleotide sequence or a combined non-native EFE expressing nucleotide sequence and non-native AKGP expressing nucleotide sequence inserted into a bacterial plasmid of the microorganism, wherein the non-native EFE expressing nucleotide sequence has a nucleotide sequence at least 95% identical to SEQ ID NO. 3 or SEQ ID NO. 4. In another embodiment, the combined non-native EFE expressing nucleotide sequence and non-native AKGP expressing nucleotide sequence has a nucleotide sequence at least 95% identical to SEQ ID NO. 7. In another embodiment, the combined non-native EFE expressing nucleotide sequence and non-native AKGP expressing nucleotide sequence expresses an amino acid sequence at least 95% identical to SEQ ID NO. 5 or SEQ ID NO. 6.

In various embodiments of producing ethylene herein, the recombinant microorganism includes a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, or a plant cell.

An embodiment of a method of producing ethylene further includes increasing an amount of ethylene production by adding at least one activator to a culture containing the recombinant microorganism located within the bioreactor culture vessel. In an embodiment, such a method includes adding CO₂ to a culture atmosphere contained within the bioreactor culture vessel at rate of between about 100 ml/minute and about 500 ml/minute. In an embodiment, such a method further includes decreasing an amount of ethylene production by removing at least one molecular switch from the cell culture containing the recombinant microorganism located within the bioreactor culture vessel. In an embodiment, such a method further includes controlling the amount of ethylene produced from the microbial culture by increasing or decreasing the concentration of at least one nutrient or the amount of at least one stimulus when culturing the recombinant microorganism. In an embodiment, the concentration of at least one nutrient and the amount of at least one stimulus are at a ratio of from about 0.5-1.5 gr./liter to about 0.1 mM in the microbial culture. In an embodiment, such a method further includes removing the amount of ethylene produced from the microbial culture by condensing the ethylene from a gaseous to a liquid state, or wherein the amount of ethylene recovered is from about 0.5 ml to about 10 ml/liter/h.

Embodiments herein are directed to a recombinant microorganism having an improved alpha-ketoglutarate (AKG) producing ability. In certain embodiments, the recombinant microorganism expresses at least one phosphoenolpyruvate synthase (PEP) protein having an amino acid sequence at least 95% identical to SEQ ID NO. 15 by expressing a non-native PEP expressing nucleotide sequence, and wherein an amount of PEP protein produced by the recombinant microorganism is greater than that produced relative to a control microorganism lacking the non-native PEP expressing nucleotide sequence.

In certain embodiments, the recombinant microorganism expresses at least one isocitrate dehydrogenase (IDH) protein having an amino acid sequence at least 95% identical to SEQ ID NO. 20 by expressing a non-native IDH expressing nucleotide sequence, and wherein an amount of IDH protein produced by the recombinant microorganism is greater than that produced relative to a control microorganism lacking the non-native IDH expressing nucleotide sequence; wherein an amount of AKG produced by the recombinant microorganism is greater than that produced relative to the control microorganism.

In certain embodiments, the recombinant microorganism expresses at least one citrate synthase protein having an amino acid sequence at least 95% identical to SEQ ID NO. 17 by expressing a non-native citrate synthase expressing nucleotide sequence, wherein an amount of citrate synthase protein produced by the recombinant microorganism is greater than that produced relative to a control microorganism lacking the non-native citrate synthase expressing nucleotide sequence.

In certain embodiments, the recombinant microorganism contains a deletion in a glucose-1-phosphate adenylyltransferase expressing nucleotide sequence, wherein an amount of glucose-1-phosphate adenylyltransferase protein produced by the recombinant microorganism is less than that produced relative to a control microorganism lacking the deletion.

In certain embodiments, the recombinant microorganism includes a microorganism selected from the group consisting of a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 is a flow chart depicting an embodiment of a method of producing ethylene herein.

FIG. 2 is an illustration of a vector plasmid for expression of an ethylene forming enzyme (EFE) protein according to embodiments herein.

FIG. 3A is a photograph of an SDS-PAGE gel showing expression of an EFE protein according to embodiments herein.

FIG. 3B is a photograph of a Western blot showing expression of an EFE protein according to embodiments herein.

FIG. 4A is a graph showing the growth rate of E. coli BL 21 PUC19 EFE over time according to embodiments herein.

FIG. 4B is a graph showing ethylene yield over time for an E. coli BL 21 PUC19 EFE culture according to embodiments herein.

FIG. 5A is a photograph showing growth of bacterial colonies according to embodiments herein.

FIG. 5B is a photograph showing growth of bacterial colonies according to embodiments herein.

FIG. 6 is a photograph of a Southern blot showing the results of a cloning experiment for AKG and sucrose production according to embodiments herein.

FIG. 7A is a photograph of a Southern blot showing the results of a cloning experiment for sucrose production according to embodiments herein.

FIG. 7B is a photograph of a flask bacterial culture according to embodiments herein.

FIG. 8A is a photograph of a Southern blot showing the results of a cloning experiment for ethylene production according to embodiments herein.

FIG. 8B is a photograph of a Southern blot showing the results of a cloning experiment for ethylene production according to embodiments herein.

FIG. 9A is a photograph of a Southern blot showing the results of a cloning experiment for AKG production according to embodiments herein.

FIG. 9B is a photograph of a flask bacterial culture according to embodiments here.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one nutrient” means one nutrient, more than one nutrient, or any combination thereof.

Unless otherwise noted, the term “about” refers to ±10% of the non-percentage number that is described, rounded to the nearest whole integer. For example, about 100 ml/minute, would include 90 to 110 ml/minute. Unless otherwise noted, the term “about” refers to ±5% of a percentage number. For example, about 95% would include 90 to 100%. When the term “about” is discussed in terms of a range, then the term refers to the appropriate amount less than the lower limit and more than the upper limit. For example, from about 100 to about 500 ml/minute would include from 90 to 550 ml/minute.

Unless otherwise noted, measurable properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any composition of matter, method or system of any embodiment herein.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by various methods, known to those skilled in the art. In an embodiment, sequence identity is determined by comparing the whole length of the sequences as identified herein.

Exemplary methods to determine identity are designed to give the largest match between the sequences tested. Methods to determine identity and similarity are codified in publicly available computer programs. Exemplary computer program methods to determine identity and similarity between two sequences include e.g. the BestFit, BLASTP (Protein Basic Local Alignment Search Tool), BLASTN (Nucleotide Basic Local Alignment Search Tool), and FASTA (Altschul, S. F. et al., J. Mol. Biol. 215:403-410 (1990), publicly available from NCBI and other sources (BLAST® Manual, Altschul, S., et al., NCBI NLM NIH Bethesda, Md. 20894). A most exemplary algorithm used is EMBOSS (European Molecular Biology Open Software Suite). Exemplary parameters for amino acid sequences comparison using EMBOSS are gap open 10.0, gap extend 0.5, Blosum matrix. Exemplary parameters for nucleic acid sequences comparison using EMBOSS are gap open 10.0, gap extend 0.5, DNA full matrix (DNA identity matrix). In embodiments, it is possible to compare the DNA/protein sequences among different species to determine the homology of sequences using online data such as Gene bank, KEG, BLAST and Ensemble.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Unless otherwise noted, the term “adapted” or “codon adapted” refers to “codon optimization” of polynucleotides as disclosed herein, the sequence of which may be native or non-native, or may be adapted for expression in other microorganisms. Codon optimization adapts the codon usage for an encoded polypeptide towards the codon bias of the organism in which the polypeptide is to be expressed. Codon optimization generally helps to increase the production level of the encoded polypeptide in the host cell.

Carbon dioxide emissions resulting from the use of fossil fuels continue to rise on a global scale. Reduction of atmospheric carbon dioxide levels is a key to mitigating or reversing climate change. Carbon capture and storage (CCS) is a prominent technology for removal of industrial carbon dioxide from the atmosphere; it has been estimated that over 20 trillion tons of carbon dioxide captured from refining and other industrial processes can be transported and stored in various types of subterranean environments or storage tanks. Although CCS is a cost effective and affordable way to reduce carbon dioxide emissions compared to other currently available methods, the problem remains that the carbon dioxide is merely being stored underground until it escapes. Therefore, CCS methods do not provide a sustainable solution to reduce excess carbon dioxide in the atmosphere. Also, there is little financial incentive for industries to pump carbon dioxide into subterranean environments, unless they are forced to by environmental regulations, or they are paid to do it as part of their business model. Arguably, global warming is a crisis because it is more lucrative to produce carbon dioxide than to dispose of carbon dioxide.

There remains a need to remove excess carbon dioxide from the atmosphere in more efficient and sustainable ways. There remains a need for technologies that can harness the over-abundance of carbon dioxide to make useful products, and for other applications that are beneficial to industry and the environment.

The challenges of the limited supply of petroleum, and the harmful effects of petroleum operations on the environment, have prompted a growing emphasis on maximizing output from existing resources, and in developing renewable sources of fuels and chemicals that can minimize environmental impacts. Since ethylene is a potentially renewable feedstock, there has been a great deal of interest in developing technologies to produce ethylene from renewable sources, such as carbon dioxide and biomass. Ethylene is the most widely produced organic compound in the world, useful in a broad spectrum of industries including plastics, solvents, and textiles. Ethylene is currently produced by steam cracking fossil fuels or dehydrogenating ethane. With millions of metric tons of ethylene being produced each year, however, more than enough carbon dioxide is produced by such processes to greatly contribute to the global carbon footprint. Producing ethylene through renewable methods would accordingly help to meet the huge demand from the energy and chemical industries, while also helping to protect the environment.

Conventional methods have been developed to produce bio-ethylene using ethanol derived from corn or sugar cane. However, the production of bio-ethylene from biomass (e.g. corn and sugar cane) is a time-consuming and cost-ineffective process, requiring land, transportation, and digestion of biomass. For example, there are massive inefficiencies associated with the growing and transportation of corn and sugar cane, which by itself causes CO₂ emission. A variety of microbes, including bacteria and fungi, naturally produce ethylene in small amounts. Such microbes make use of an ethylene-forming enzyme (EFE). A type of ethylene pathway, such as is found in Pseudomonas syringae and Penicillium digitatum, uses alpha-ketoglutarate (AKG) and arginine as substrates in a reaction catalyzed by an ethylene-forming enzyme. Ethylene-forming enzymes provide a promising target, because expression of a single gene can be sufficient for ethylene production. Techniques making use of heterologous expression of an EFE have been demonstrated in several microbial species, where the microbial hosts have been able to utilize a variety of carbon sources in the Calvin cycle, including lignocellulose and carbon dioxide. Plus, recent developments in cost-effective high throughput genetic sequencing technologies have led to an increased understanding of microbial gene expression. However, the currently available technologies do not produce industrially relevant quantities of ethylene through microbial activity. There remains a need for improvements in microbial bio-ethylene production that can produce ethylene at a commercial scale. There remains a need for methods to produce ethylene useful for industrial and other applications using carbon dioxide feedstocks.

Embodiments of the present disclosure can provide a benefit not only of removing carbon dioxide from the environment along with the benefit of producing a valuable organic compound capable of being sold commercially. Embodiments of the present disclosure can thus provide a renewable alternative to conventional carbon dioxide storage, by using recombinant microbial technology to convert the carbon dioxide into ethylene as a useful organic compound. One benefit of the embodiments of the present disclosure is that the methods can make it economically profitable for an oil or natural gas company to remove carbon dioxide from the environment. An oil company, or a contractor thereof, could instead of pumping carbon dioxide into a subterranean environment or leaving the sequestered carbon dioxide underground, use the carbon dioxide as a carbon source for a culture of recombinant microorganisms to convert the carbon dioxide to ethylene in a cost-effective way. Also, much the carbon dioxide generated by transportation can be avoided because the process can be practiced on-site or would be expected to consumer more carbon dioxide than it produces.

The most effective methods for protecting the environment are those methods that people actually use. The more profitable those methods are; the more likely people are to use them. One of the benefits of the methods disclosed herein is the cost-effectiveness of using a bioreactor system. Embodiments of the present disclosure can provide a benefit of engineering a photosynthetic ethylene producing microorganism, by adapting the relevant metabolic signaling pathways to produce ethylene on an industrial scale. Such embodiments can make it profitable to remove carbon dioxide from the atmosphere and to passively generate valuable organic compounds while the microbes do the work—on a scale previously unimaginable.

What would happen to the global warming crisis if it became more profitable, or just as profitable, to convert carbon dioxide into valuable organic compounds as it did to generate the carbon dioxide in the first place? The presently disclosed methods might transform energy producers from global warming companies to global cooling companies.

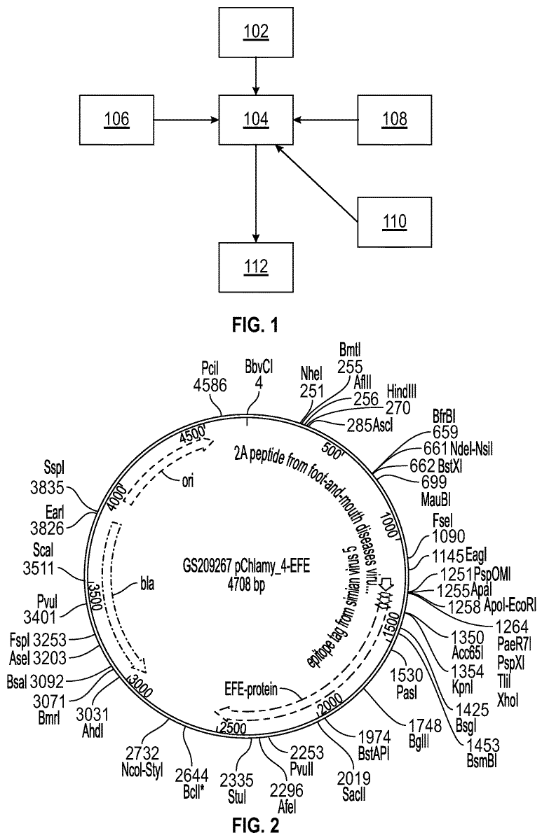

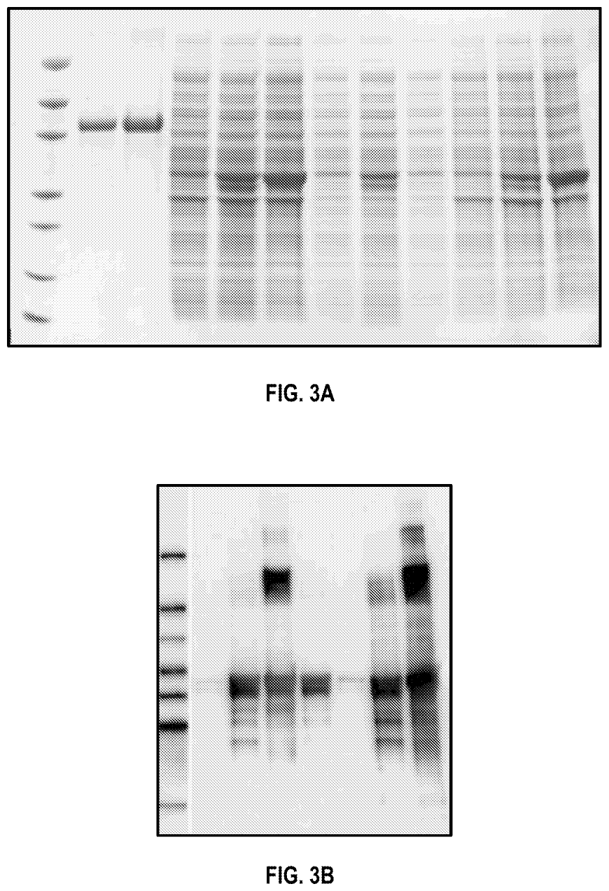

The present disclosure relates to recombinant microorganisms having an improved ethylene producing ability. The present disclosure relates to methods of producing ethylene, including providing a recombinant microorganism having an improved ethylene producing ability according to various embodiments herein. As a general overview of a method disclosed herein, referring to FIG. 1, the method includes providing a recombinant microorganism expressing at least one EFE protein according to embodiments disclosed herein 102; culturing the recombinant microorganism in a bioreactor culture vessel under conditions sufficient to produce ethylene in the bioreactor culture vessel 104; increasing an amount of ethylene production by adding at least one activator to the culture within the bioreactor culture vessel, or adding carbon dioxide to a culture atmosphere within the bioreactor culture vessel 106; decreasing an amount of ethylene production by removing at least one molecular switch from the cell culture 108; controlling an amount of ethylene produced from the microbial culture by increasing or decreasing the concentration of at least one nutrient or the amount of at least one stimulus when culturing the recombinant microorganism 110; and removing an amount of ethylene produced from the microbial culture by condensing the ethylene from a gaseous to a liquid state 112. As an illustration of a vector plasmid for expression of an EFE protein according to embodiments herein, referring to FIG. 2, a non-native EFE expressing nucleotide sequence is inserted into the vector plasmid of a Chlamydomonas sp. bacterium. As an illustration of a recombinant microorganism having an improved ethylene producing ability herein, referring to the illustration of an SDS-PAGE gel in FIG. 3A and the illustration of a Western blot in FIG. 3B, an EFE protein is expressed from a vector plasmid of an Escherichia sp. bacterium having a non-native EFE expressing nucleotide sequence inserted into the vector plasmid, as shown by the arrows.

Embodiments of Recombinant Microorganisms

The present disclosure relates to a recombinant microorganism having an improved ethylene producing ability. In such embodiments, the recombinant microorganism expresses at least one ethylene forming enzyme (EFE) protein having an amino acid sequence at least 95% identical to SEQ ID NO: 1 by expressing a non-native EFE expressing nucleotide sequence. In some embodiments, the non-native EFE expressing nucleotide sequence encodes an EFE of Pseudomonas savastanoi. In an embodiment, the EFE protein has an amino acid sequence at least 80% or at least 90% identical to SEQ ID NO: 1. In an embodiment, the EFE protein has an amino acid sequence at least 98% identical to SEQ ID NO: 1. In various embodiments, an amount of EFE protein produced by the recombinant microorganism is greater than that produced relative to a control microorganism lacking the non-native EFE expressing nucleotide sequence. In some embodiments, the amount of EFE protein produced by the recombinant microorganism is from about 5% to about 200% or more greater than that produced relative to the control microorganism lacking the non-native EFE expressing nucleotide sequence. In some embodiments, the amount of EFE protein produced by the recombinant microorganism is from about 50% to about 150% or more greater than that produced relative to the control microorganism lacking the non-native EFE expressing nucleotide sequence. In some embodiments, the amount of EFE protein produced by the recombinant microorganism is from about 75% to about 100% or more greater than that produced relative to the control microorganism lacking the non-native EFE expressing nucleotide sequence.

In an embodiment, the recombinant microorganism also expresses at least one alpha-ketoglutarate permease (AKGP) protein by expressing a non-native AKGP expressing nucleotide sequence. In an embodiment, the AKGP protein has an amino acid sequence at least 95% identical to SEQ ID NO: 2. In an embodiment, the AKGP protein has an amino acid sequence at least 80% or at least 90% identical to SEQ ID NO: 2. In an embodiment, the AKGP protein has an amino acid sequence at least 98% identical to SEQ ID NO: 2. In an embodiment, the original sequence for SEQ ID NO: 2 was from AKGP from Pseudomonas syringe, but sequence innovation was performed to improve the expression of this sequence in Synechococcus elongatus. In various embodiments, an amount of AKGP protein produced by the recombinant microorganism is greater than that produced relative to a control microorganism lacking the non-native AKGP expressing nucleotide sequence. In some embodiments, the amount of AKGP protein produced by the recombinant microorganism is from about 5% to about 200% or more greater than that produced relative to the control microorganism lacking the non-native AKGP expressing nucleotide sequence. In some embodiments, the amount of AKGP protein produced by the recombinant microorganism is from about 50% to about 150% or more greater than that produced relative to the control microorganism lacking the non-native AKGP expressing nucleotide sequence. In some embodiments, the amount of AKGP protein produced by the recombinant microorganism is from about 75% to about 100% or more greater than that produced relative to the control microorganism lacking the non-native AKGP expressing nucleotide sequence.

In various embodiments, the recombinant microorganism includes a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, or a plant cell. In some embodiments, the recombinant microorganism can include Saccharomyces cerevisiae, Pseudomonas putida, Trichoderma viride, Trichoderma reesei, and tobacco.

In an embodiment, the non-native EFE expressing nucleotide sequence is inserted into a bacterial vector plasmid, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 3, and the non-native EFE expressing nucleotide sequence is inserted into a vector plasmid of a Chlamydomonas sp. bacterium. In an embodiment, the non-native EFE expressing nucleotide sequence has a nucleotide sequence at least 90% identical to SEQ ID NO. 3. In an embodiment, the non-native EFE expressing nucleotide sequence has a nucleotide sequence at least 98% identical to SEQ ID NO. 3. In an embodiment, the non-native EFE expressing nucleotide sequence is codon adapted for expression in a Chlamydomonas sp. bacterium.

In an embodiment, a non-native EFE expressing nucleotide sequence and a non-native AKGP expressing nucleotide sequence are inserted into a bacterial vector plasmid, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 4, and the non-native EFE expressing nucleotide sequence and the AKGP expressing nucleotide sequence are inserted into a vector plasmid of an Escherichia sp. bacterium. In an embodiment, the non-native EFE expressing nucleotide sequence has a nucleotide sequence at least 90% identical to SEQ ID NO. 4. In an embodiment, the non-native EFE expressing nucleotide sequence has a nucleotide sequence at least 98% identical to SEQ ID NO. 4. In an embodiment, the non-native EFE expressing nucleotide sequence is codon adapted for expression in an Escherichia sp. bacterium, or in an Escherichia coli bacterium.

In an embodiment, the recombinant microorganism further includes a non-native AKGP expressing nucleotide sequence. In some such embodiments, the non-native EFE expressing nucleotide sequence and non-native AKGP expressing nucleotide sequence express a combined amino acid sequence at least 95% identical to SEQ ID NO. 5. In some embodiments, the non-native expressing nucleotide sequence and non-native AKGP expression nucleotide sequence express a combined amino acid sequence at least 95% identical to SEQ ID NO. 6. In such embodiments, the combined amino acid sequence can include one or more purification tags; in an embodiment, the purification tag includes a histidine tag. In an embodiment, the purification tag includes a His-TEV sequence; in an embodiment, the His-TEV sequence includes SEQ ID NO. 10. In other embodiments, the non-native EFE expressing nucleotide sequence and non-native AKGP expressing nucleotide sequence express a combined amino acid sequence at least 90% identical to SEQ ID NO. 5 or SEQ ID NO. 6. In other embodiments, the non-native EFE expressing nucleotide sequence and non-native AKGP expressing nucleotide sequence express a combined amino acid sequence at least 98% identical to SEQ ID NO. 5 or SEQ ID NO. 6. In some embodiments, the non-native EFE expressing nucleotide sequence and non-native AKGP expressing nucleotide sequence are inserted into a bacterial plasmid of a Synechococcus sp. bacterium. In an embodiment, the non-native EFE expressing nucleotide sequence and non-native AKGP expressing nucleotide sequence include a nucleotide sequence at least 95% identical to SEQ ID NO. 7. In an embodiment, the non-native EFE expressing nucleotide sequence and non-native AKGP expressing nucleotide sequence include a nucleotide sequence at least 90% identical to SEQ ID NO. 7. In an embodiment, the non-native EFE expressing nucleotide sequence and non-native AKGP expressing nucleotide sequence include a nucleotide sequence at least 98% identical to SEQ ID NO. 7. In an embodiment, the non-native EFE expressing nucleotide sequence and the non-native AKGP expressing nucleotide sequence are codon adapted for expression in a Cyanobacteria. In an embodiment, the non-native EFE expressing nucleotide sequence and the non-native AKGP expressing nucleotide sequence are codon adapted for expression in a Synechococcus sp. bacterium.

Embodiments of Methods of Producing a Recombinant Microorganism

Embodiments herein are directed to methods of producing a recombinant microorganism having an improved ethylene producing ability. In an embodiment, the method includes producing a recombinant microorganism by inserting a non-native EFE expressing nucleotide sequence into a bacterial plasmid of a microorganism. In some embodiments, the non-native EFE expressing nucleotide sequence encodes an EFE of Pseudomonas savastanoi. In an embodiment, the non-native EFE expressing nucleotide sequence has a nucleotide sequence at least 95% identical to SEQ ID NO. 3. In an embodiment, the non-native EFE expressing nucleotide sequence has a nucleotide sequence at least 90% identical to SEQ ID NO. 3. In an embodiment, the non-native EFE expressing nucleotide sequence has a nucleotide sequence at least 98% identical to SEQ ID NO. 3. In an embodiment, the non-native EFE expressing nucleotide sequence is codon adapted for expression in a Chlamydomonas sp. bacterium. In an embodiment, the Chlamydomonas sp. bacterium includes Chlamydomonas reinhardtii.

In an embodiment, the non-native EFE expressing nucleotide sequence has a nucleotide sequence at least 95% identical to SEQ ID NO. 4. In an embodiment, the non-native EFE expressing nucleotide sequence has a nucleotide sequence at least 90% identical to SEQ ID NO. 4. In an embodiment, the non-native EFE expressing nucleotide sequence has a nucleotide sequence at least 98% identical to SEQ ID NO. 4. In an embodiment, the non-native EFE expressing nucleotide sequence is codon adapted for expression in an Escherichia sp. bacterium. In an embodiment, the Escherichia sp. bacterium includes E. coli. In an embodiment, the non-native EFE expressing nucleotide sequence includes an N-terminal NdeI cloning site (SEQ ID NO. 8 (See Appendix)). In an embodiment, the non-native EFE expressing nucleotide sequence includes one or more purification tags; in an embodiment, the purification tag includes a histidine tag. In an embodiment, the purification tag includes a histidine tag at the C-terminal end, followed by a stop codon and a HindIII cloning site (SEQ ID NO. 9 (See Appendix)).

In an embodiment, the method includes producing a recombinant microorganism by inserting a combined non-native EFE expressing nucleotide sequence and non-native AKGP expressing nucleotide sequence into a bacterial plasmid of a microorganism. In one such embodiment, the combined non-native EFE expressing nucleotide sequence and non-native AKGP expressing nucleotide sequence expresses an amino acid sequence at least 95% identical to SEQ ID NO. 5. In some embodiments, the non-native EFE expressing nucleotide sequence and non-native AKGP expression nucleotide sequence express a combined amino acid sequence at least 95% identical to SEQ ID NO. 6. In such embodiments, the combined amino acid sequence can include one or more purification tags; in an embodiment, the purification tag includes a histidine tag. In an embodiment, the purification tag includes a His-TEV sequence; in an embodiment, the His-TEV sequence includes SEQ ID NO. 10 (See Appendix). In other embodiments, the non-native EFE expressing nucleotide sequence and non-native AKGP expressing nucleotide sequence express a combined amino acid sequence at least 90% identical to SEQ ID NO. 5 or SEQ ID NO. 6. In other embodiments, the non-native EFE expressing nucleotide sequence and non-native AKGP expressing nucleotide sequence express a combined amino acid sequence at least 98% identical to SEQ ID NO. 5 or SEQ ID NO. 6. In some embodiments, the non-native EFE expressing nucleotide sequence and non-native AKGP expressing nucleotide sequence are inserted into a bacterial plasmid of a Synechococcus sp. bacterium. In an embodiment, the non-native EFE expressing nucleotide sequence and non-native AKGP expressing nucleotide sequence include a nucleotide sequence at least 95% identical to SEQ ID NO. 7. In an embodiment, the non-native EFE expressing nucleotide sequence and non-native AKGP expressing nucleotide sequence include a nucleotide sequence at least 90% identical to SEQ ID NO. 7. In an embodiment, the non-native EFE expressing nucleotide sequence and non-native AKGP expressing nucleotide sequence include a nucleotide sequence at least 98% identical to SEQ ID NO. 7. In an embodiment, the non-native EFE expressing nucleotide sequence and the non-native AKGP expressing nucleotide sequence are codon adapted for expression in a Cyanobacteria. In an embodiment, the non-native EFE expressing nucleotide sequence and the non-native AKGP expressing nucleotide sequence are codon adapted for expression in a Synechococcus sp. bacterium.

In various embodied methods, the microorganism includes a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, or a plant cell. In some embodiments, the recombinant microorganism can include Saccharomyces cerevisiae, Pseudomonas putida, Trichoderma viride, Trichoderma reesei, and tobacco.

In embodiments of methods herein, the non-native EFE expressing nucleotide sequence is inserted into a bacterial vector plasmid, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 3, and the non-native EFE expressing nucleotide sequence is inserted into a vector plasmid of a Chlamydomonas sp. bacterium. In an embodiment, the non-native EFE expressing nucleotide sequence has a nucleotide sequence at least 90% identical to SEQ ID NO. 3. In an embodiment, the non-native EFE expressing nucleotide sequence has a nucleotide sequence at least 98% identical to SEQ ID NO. 3. In an embodiment, the non-native EFE expressing nucleotide sequence is codon adapted for expression in a Chlamydomonas sp. bacterium.

In an embodiment, a non-native EFE expressing nucleotide sequence and a non-native AKGP expressing nucleotide sequence are inserted into a bacterial vector plasmid, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 4, and the non-native EFE expressing nucleotide sequence and the AKGP expressing nucleotide sequence are inserted into a vector plasmid of an Escherichia sp. bacterium. In an embodiment, the non-native EFE expressing nucleotide sequence has a nucleotide sequence at least 90% identical to SEQ ID NO. 4. In an embodiment, the non-native EFE expressing nucleotide sequence has a nucleotide sequence at least 98% identical to SEQ ID NO. 4. In an embodiment, the non-native EFE expressing nucleotide sequence is codon adapted for expression in an Escherichia sp. bacterium, or in an Escherichia coli bacterium.

In an embodiment, the recombinant microorganism further includes a non-native AKGP expressing nucleotide sequence. In some such embodiments, the non-native EFE expressing nucleotide sequence and non-native AKGP expressing nucleotide sequence express a combined amino acid sequence at least 95% identical to SEQ ID NO. 5. In some embodiments, the non-native expressing nucleotide sequence and non-native AKGP expression nucleotide sequence express a combined amino acid sequence at least 95% identical to SEQ ID NO. 6. In such embodiments, the combined amino acid sequence can include one or more purification tags; in an embodiment, the purification tag includes a histidine tag. In an embodiment, the purification tag includes a His-TEV sequence; in an embodiment, the His-TEV sequence includes SEQ ID NO. 10. In other embodiments, the non-native EFE expressing nucleotide sequence and non-native AKGP expressing nucleotide sequence express a combined amino acid sequence at least 90% identical to SEQ ID NO. 5 or SEQ ID NO. 6. In other embodiments, the non-native EFE expressing nucleotide sequence and non-native AKGP expressing nucleotide sequence express a combined amino acid sequence at least 98% identical to SEQ ID NO. 5 or SEQ ID NO. 6. In some embodiments, the non-native EFE expressing nucleotide sequence and non-native AKGP expressing nucleotide sequence are inserted into a bacterial plasmid of a Synechococcus sp. bacterium. In an embodiment, the non-native EFE expressing nucleotide sequence and non-native AKGP expressing nucleotide sequence include a nucleotide sequence at least 95% identical to SEQ ID NO. 7. In an embodiment, the non-native EFE expressing nucleotide sequence and non-native AKGP expressing nucleotide sequence include a nucleotide sequence at least 90% identical to SEQ ID NO. 7. In an embodiment, the non-native EFE expressing nucleotide sequence and non-native AKGP expressing nucleotide sequence include a nucleotide sequence at least 98% identical to SEQ ID NO. 7. In an embodiment, the non-native EFE expressing nucleotide sequence and the non-native AKGP expressing nucleotide sequence are codon adapted for expression in a Cyanobacteria. In an embodiment, the non-native EFE expressing nucleotide sequence and the non-native AKGP expressing nucleotide sequence are codon adapted for expression in a Synechococcus sp. bacterium.

Embodiments of Methods of Producing Ethylene

Methods of producing ethylene are embodied herein. An embodiment of such a method includes providing a recombinant microorganism having an improved ethylene producing ability. In an embodiment, the recombinant microorganism expresses at least one ethylene forming enzyme (EFE) protein having an amino acid sequence at least 95% identical to SEQ ID NO: 1 by expressing a non-native EFE expressing nucleotide sequence, wherein an amount of EFE protein produced by the recombinant microorganism is greater than that produced relative to a control microorganism lacking the non-native EFE expressing nucleotide sequence; culturing the recombinant microorganism in a bioreactor culture vessel under conditions sufficient to produce ethylene in the bioreactor culture vessel; and harvesting ethylene from the bioreactor culture vessel.

In some embodiments of methods of producing ethylene, the non-native EFE expressing nucleotide sequence encodes an EFE of Pseudomonas savastanoi. In an embodiment, the EFE protein has an amino acid sequence at least 80% or at least 90% identical to SEQ ID NO: 1. In an embodiment, the EFE protein has an amino acid sequence at least 98% identical to SEQ ID NO: 1. In various embodiments, an amount of EFE protein produced by the recombinant microorganism is greater than that produced relative to a control microorganism lacking the non-native EFE expressing nucleotide sequence. In some embodiments, the amount of EFE protein produced by the recombinant microorganism is from about 5% to about 200% or more greater than that produced relative to the control microorganism lacking the non-native EFE expressing nucleotide sequence. In some embodiments, the amount of EFE protein produced by the recombinant microorganism is from about 50% to about 150% or more greater than that produced relative to the control microorganism lacking the non-native EFE expressing nucleotide sequence. In some embodiments, the amount of EFE protein produced by the recombinant microorganism is from about 75% to about 100% or more greater than that produced relative to the control microorganism lacking the non-native EFE expressing nucleotide sequence.

In an embodiment of methods of producing ethylene, the non-native EFE expressing nucleotide sequence has a nucleotide sequence at least 95% identical to SEQ ID NO. 3. In an embodiment, the non-native EFE expressing nucleotide sequence has a nucleotide sequence at least 80% or at least 90% identical to SEQ ID NO. 3. In an embodiment, the non-native EFE expressing nucleotide sequence has a nucleotide sequence at least 98% identical to SEQ ID NO. 3. In an embodiment, the non-native EFE expressing nucleotide sequence is codon adapted for expression in a Chlamydomonas sp. bacterium. In an embodiment, the Chlamydomonas sp. bacterium includes Chlamydomonas reinhardtii.

In an embodiment of methods herein, the non-native EFE expressing nucleotide sequence has a nucleotide sequence at least 95% identical to SEQ ID NO. 4. In an embodiment, the non-native EFE expressing nucleotide sequence has a nucleotide sequence at least 90% identical to SEQ ID NO. 4. In an embodiment, the non-native EFE expressing nucleotide sequence has a nucleotide sequence at least 98% identical to SEQ ID NO. 4. In an embodiment, the non-native EFE expressing nucleotide sequence is codon adapted for expression in an Escherichia sp. bacterium. In an embodiment, the Escherichia sp. bacterium includes E. coli. In an embodiment, the non-native EFE expressing nucleotide sequence includes an N-terminal NdeI cloning site (SEQ ID NO. 8). In an embodiment, the non-native EFE expressing nucleotide sequence includes one or more purification tags; in an embodiment, the purification tag includes a histidine tag. In an embodiment, the purification tag includes a histidine tag at the C-terminal end, followed by a stop codon and a HindIII cloning site (SEQ ID NO. 9).

In an embodiment, the method of producing ethylene includes producing a recombinant microorganism by inserting a combined non-native EFE expressing nucleotide sequence and non-native AKGP expressing nucleotide sequence into a bacterial plasmid of a microorganism. In one such embodiment, the combined non-native EFE expressing nucleotide sequence and non-native AKGP expressing nucleotide sequence expresses an amino acid sequence at least 95% identical to SEQ ID NO. 5. In some embodiments, the non-native EFE expressing nucleotide sequence and non-native AKGP expression nucleotide sequence express a combined amino acid sequence at least 95% identical to SEQ ID NO. 6. In such embodiments, the combined amino acid sequence can include one or more purification tags; in an embodiment, the purification tag includes a histidine tag. In an embodiment, the purification tag includes a His-TEV sequence; in an embodiment, the His-TEV sequence includes SEQ ID NO. 10. In other embodiments, the non-native EFE expressing nucleotide sequence and non-native AKGP expressing nucleotide sequence express a combined amino acid sequence at least 90% identical to SEQ ID NO. 5 or SEQ ID NO. 6. In other embodiments, the non-native EFE expressing nucleotide sequence and non-native AKGP expressing nucleotide sequence express a combined amino acid sequence at least 98% identical to SEQ ID NO. 5 or SEQ ID NO. 6. In some embodiments, the non-native EFE expressing nucleotide sequence and non-native AKGP expressing nucleotide sequence are inserted into a bacterial plasmid of a Synechococcus sp. bacterium. In an embodiment, the non-native EFE expressing nucleotide sequence and non-native AKGP expressing nucleotide sequence include a nucleotide sequence at least 95% identical to SEQ ID NO. 7. In an embodiment, the non-native EFE expressing nucleotide sequence and non-native AKGP expressing nucleotide sequence include a nucleotide sequence at least 90% identical to SEQ ID NO. 7. In an embodiment, the non-native EFE expressing nucleotide sequence and non-native AKGP expressing nucleotide sequence include a nucleotide sequence at least 98% identical to SEQ ID NO. 7. In an embodiment, the non-native EFE expressing nucleotide sequence and the non-native AKGP expressing nucleotide sequence are codon adapted for expression in a Cyanobacteria. In an embodiment, the non-native EFE expressing nucleotide sequence and the non-native AKGP expressing nucleotide sequence are codon adapted for expression in a Synechococcus sp. bacterium.

In various embodied methods, the microorganism includes a Cyanobacteria, a Synechococcus, Synechococcus elongatus, Synechococcus leopoliensis, Synechocystis, Anabaena, a Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, or a plant cell. In some embodiments, the recombinant microorganism can include Saccharomyces cerevisiae, Pseudomonas putida, Trichoderma viride, Trichoderma reesei, and tobacco.

In embodiments of methods herein, the non-native EFE expressing nucleotide sequence is inserted into a bacterial vector plasmid, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 3, and the non-native EFE expressing nucleotide sequence is inserted into a vector plasmid of a Chlamydomonas sp. bacterium. In an embodiment, the non-native EFE expressing nucleotide sequence has a nucleotide sequence at least 90% identical to SEQ ID NO. 3. In an embodiment, the non-native EFE expressing nucleotide sequence has a nucleotide sequence at least 98% identical to SEQ ID NO. 3. In an embodiment, the non-native EFE expressing nucleotide sequence is codon adapted for expression in a Chlamydomonas sp. bacterium.

In an embodiment, a non-native EFE expressing nucleotide sequence and a non-native AKGP expressing nucleotide sequence are inserted into a bacterial vector plasmid, a nucleotide guide of a homologous recombination system, a CRISPR CAS system, a phage display system, or a combination thereof. In an embodiment, the non-native EFE expressing nucleotide sequence has a nucleotide sequence at least 95% identical to SEQ ID NO. 4, and the non-native EFE expressing nucleotide sequence and the AKGP expressing nucleotide sequence are inserted into a vector plasmid of an Escherichia sp. bacterium. In an embodiment, the non-native EFE expressing nucleotide sequence has a nucleotide sequence at least 90% identical to SEQ ID NO. 4. In an embodiment, the non-native EFE expressing nucleotide sequence has a nucleotide sequence at least 98% identical to SEQ ID NO. 4. In an embodiment, the non-native EFE expressing nucleotide sequence is codon adapted for expression in an Escherichia sp. bacterium, or in an Escherichia coli bacterium.

In an embodiment, the recombinant microorganism further includes a non-native AKGP expressing nucleotide sequence. In some such embodiments, the non-native EFE expressing nucleotide sequence and non-native AKGP expressing nucleotide sequence express a combined amino acid sequence at least 95% identical to SEQ ID NO. 5. In some embodiments, the non-native expressing nucleotide sequence and non-native AKGP expression nucleotide sequence express a combined amino acid sequence at least 95% identical to SEQ ID NO. 6. In such embodiments, the combined amino acid sequence can include one or more purification tags; in an embodiment, the purification tag includes a histidine tag. In an embodiment, the purification tag includes a His-TEV sequence; in an embodiment, the His-TEV sequence includes SEQ ID NO. 10. In other embodiments, the non-native EFE expressing nucleotide sequence and non-native AKGP expressing nucleotide sequence express a combined amino acid sequence at least 90% identical to SEQ ID NO. 5 or SEQ ID NO. 6. In other embodiments, the non-native EFE expressing nucleotide sequence and non-native AKGP expressing nucleotide sequence express a combined amino acid sequence at least 98% identical to SEQ ID NO. 5 or SEQ ID NO. 6. In some embodiments, the non-native EFE expressing nucleotide sequence and non-native AKGP expressing nucleotide sequence are inserted into a bacterial plasmid of a Synechococcus sp. bacterium. In an embodiment, the non-native EFE expressing nucleotide sequence and non-native AKGP expressing nucleotide sequence include a nucleotide sequence at least 95% identical to SEQ ID NO. 7. In an embodiment, the non-native EFE expressing nucleotide sequence and non-native AKGP expressing nucleotide sequence include a nucleotide sequence at least 90% identical to SEQ ID NO. 7. In an embodiment, the non-native EFE expressing nucleotide sequence and non-native AKGP expressing nucleotide sequence include a nucleotide sequence at least 98% identical to SEQ ID NO. 7. In an embodiment, the non-native EFE expressing nucleotide sequence and the non-native AKGP expressing nucleotide sequence are codon adapted for expression in a Cyanobacteria. In an embodiment, the non-native EFE expressing nucleotide sequence and the non-native AKGP expressing nucleotide sequence are codon adapted for expression in a Synechococcus sp. bacterium.

Embodiments of producing ethylene herein include culturing a recombinant microorganism in a bioreactor culture under conditions sufficient to produce ethylene in the bioreactor culture vessel. A bioreactor culture according to embodied methods can include one or more suitable reagents or growth media for supporting the growth of the recombinant microorganism culture. Such reagents or culture media can include water, one or more carbohydrates, one or more amino acids or amino acid derivatives, one or more buffers, sea water, Luria broth, Luria Bertani broth, BG-11 media, carbon dioxide, light, temperature, electricity, or combinations thereof.

An embodiment of a method of producing ethylene includes increasing an amount of ethylene production by adding at least one activator to a culture containing the recombinant microorganism located within the bioreactor culture vessel. The addition of such an activator can include increasing a concentration of one or more substrates of the EFE enzyme being expressed by the recombinant microorganism culture. Such a substrate can include alpha-ketoglutarate or arginine, or combinations thereof as well as other sources of carbon such as glycerol and glucose. In other embodiments, adding at least one activator can include adding a molecular switch. In some embodiments, adding at least one activator can include insertion of an inducible promoter upstream of the EFE gene; one such promoter includes an IPTG promoter. In such embodiments, IPTG can be added as a molecular switch to the culture media. In some embodiments, adding at least one activator can include adding one or more nutrients or stimuli to the culture. Such nutrients or stimuli can include one or more carbohydrates, one or more amino acids or amino acid derivatives, one or more EFE substrates, succinate, carbon dioxide, light, temperature, electricity, glycerol, sugars, or combinations thereof. In such embodiments, adding at least one activator to the culture can provide a benefit of controlling the cycles of ethylene production and enhancing the ethylene production rate. In some embodiments, the ethylene produced can be removed from the bioreactor culture vessel as it is produced. In such embodiments, removal of the ethylene can include condensing ethylene produced as a gas into a liquid form for removal from the bioreactor culture vessel.

In an embodiment, a method of producing ethylene includes adding CO₂ to a culture atmosphere contained within the bioreactor culture vessel at rate of between about 100 ml/minute and about 500 ml/minute. In an embodiment, the method includes adding CO₂ to a culture atmosphere contained within the bioreactor culture vessel at rate of between about 150 ml/minute and about 450 ml/minute. In an embodiment, the method includes adding CO₂ to a culture atmosphere contained within the bioreactor culture vessel at rate of between about 250 ml/minute and about 350 ml/minute. Such embodiments can provide a benefit of enhancing or controlling the rate of ethylene production in the bioreactor culture vessel, as well as providing a benefit of converting CO₂ into a useful product.

In an embodiment, a method of producing ethylene includes decreasing an amount of ethylene production by removing at least one molecular switch from the microbial culture containing the recombinant microorganism located within the bioreactor culture vessel. In an embodiment, such a method further includes controlling the amount of ethylene produced from the microbial culture by increasing or decreasing the concentration of at least one nutrient or the amount of at least one stimulus when culturing the recombinant microorganism. In an embodiment, the concentration of at least one nutrient and the amount of at least one stimulus are at a ratio of from about 0.5-1.5 gr./liter to about 0.1 mM in the microbial culture. In an embodiment, such a method further includes removing the amount of ethylene produced from the microbial culture by condensing the ethylene from a gaseous to a liquid state, or wherein the amount of ethylene recovered is from about 0.5 ml to about 10 ml/liter/h. Such embodiments can provide a benefit of controlling the amount of ethylene production by controlling the rate of activity of the Calvin cycle in the microbial culture. For example, it is possible to shift from an expression system to a growth system, where the cells are allowed to grow for 5-7 days and their growth conditions are monitored. When the cells reach an exponential growth condition (meaning that the cells are metabolically active), it is possible to shift from the growth system to an expression system, where the cells are shifted to an ethylene production cycle to produce ethylene for harvesting. This expression system might be maintained for 7 or more days.

### EXAMPLES

### Example 1. Cloning of Ethylene Forming Enzyme Gene Sequence into Chlamydomonas reinhardtii Vector Plasmid

An EFE (Ethylene Forming Enzyme) protein will be expressed and produced in Chlamydomonas reinhardtii. Plasmid pChlamy_4-EFE was generated successfully, to be used in EFE protein expression (Creative Enzymes, Shirley, N.Y.).

The polynucleotide coding for the Pseudomonas savastanoi pv. Phaseolicola EFE protein (GenBank: KPB44727.1, SEQ ID NO: 1) was cloned into the pChlamy_4 vector plasmid (ThermoFisher). Other reagents and use of instruments were provided by Creative Biostructure.

The Ethylene-forming enzyme (EFE) gene sequence from strain Pseudomonas savastanoi pv. Phaseolicola (GenBank: KPB44727.1) was used for the preparation of EFE recombinant protein. The corresponding nucleotide sequences were codon adapted for expression in Chlamydomonas reinhardtii and synthesized (SEQ ID NO: 3). The EFE construct was cloned into the pChlamy_4 vector with the Kpnl and Pstl restriction enzyme sites.

According to the pChlamy_4vector character, EFE with an N-tag was designed and generated. The pChlamy_4 vector contains the ATG initiation codon (vector ATG) for proper initiation of translation at position 497-499, found at the beginning of the Sh ble gene after the removal of Intron-1 Rbc S2. The FMDV 2A peptide gene flanking the Multiple Cloning Site 1 (MCS1) is in frame with the Sh ble gene. To use the N-terminal 6×His-V5-TEV tag, the EFE sequence was cloned in-frame after the TEV site, into the KphI/PstI digested pChlamy_4 vector. A TAA (stop codon) was designed for proper translation termination. The resulting sequence chromatogram is shown in FIG. 2; referring to FIG. 2, the EFE protein gene coding sequences are shown in the arrow labeled “EFE-protein”. An open reading frame orientation was confirmed by plasmid validation by nucleotide sequencing.

### Example 2: Protein Expression Evaluation of Expression of EFE in E. coli

The polynucleotide coding for the Pseudomonas savastanoi pv. Phaseolicola EFE protein (GenBank: KPB44727.1, SEQ ID NO: 1) was cloned into the pET-30a(+) vector plasmid. The corresponding nucleotides sequences were codon adapted for expression in E. coli (SEQ ID NO: 4), containing an optional His tag at the C-terminal end followed by a stop codon and HindII site (SEQ ID NO: 9). An NdeI site was used for cloning at the 5-prime end, where the NdeI site contains an ATG start codon (SEQ ID NO: 8). E. coli BL21(DE3) competent cells were transformed with the recombinant plasmid. A single colony was inoculated into LB medium containing kanamycin; cultures were incubated in 37° C. at 200 rpm. Once cell density reached to OD=0.6-0.8 at 600 nm, 0.5 mM IPTG was introduced for induction. A pilot expression of EFE (about 44.5 kDa)_BL21(DE3) was conducted. SDS-PAGE (FIG. 3A) and Western blotting (FIG. 3B) were used to monitor the EFE protein expression (GenScript USA, Inc., Piscataway, N.J.). Referring to FIG. 3A and FIG. 3B:

SDS PAGE (left) and Western blot (right, using anti His antibody (GenScript, Cat. No. A00186)) analysis of Pilot expression of EFE in E. coli expression in construct pET 30a(+).

Lane M₁: Protein marker
Lane M₂: Western blot marker

### Lane PC₁: BSA (1 μg)

### Lane PC₂: BSA (2 μg)

Lane NC: Cell lysate without induction
Lane 1: Cell lysate with induction for 16 h at 15° C.
Lane 2: Cell lysate with induction for 4 h at 37° C.
Lane NC₁: Supernatant of cell lysate without induction
Lane 3: Supernatant of cell lysate with induction for 16 h at 15 C
Lane 4: Supernatant of cell lysate with induction for 4 h at 37 C
Lane NC₂: Pellet of cell lysate without induction
Lane 5: Pellet of cell lysate with induction for 16 h at 15° C.
Lane 6: Pellet of cell lysate with induction for 4 h at 37° C.

The results of SDS-PAGE and Western blots showed that EFE was expressed in E. coli. The highest EFE expression conditions were found with induction for 16 h at 15° C. that resulted in an expression level of 5 mg/L and a solubility of 30%.

### Example 3: Recombinant EFE and AKGP Expressing Nucleotide Sequences Adapted for Expression in Synechococcus spp. Bacteria

A combined polynucleotide sequence (EFE_-P2A-aKGP, SEQ ID NO: 7) for expressing the EFE protein and for expressing the AKGP protein (SEQ ID NO. 2) was generated, after adaptation of the nucleotide sequence for expression in Cyanobacteria species Synechococcus elongates and Synechococcus leopoliensis (GenScript, Piscataway, N.J.). An codon adaptation analysis algorithm was used which adapts a variety of parameters that are critical to the efficiency of gene expression, including but not limited to codon usage bias, GC content, CpG dinucleotide content, mRNA secondary structure, cryptic splicing sites, premature PolyA sites, internal chi sites and ribosomal binding sites, negative CpG islands, RNA instability motif (ARE), repeat sequences (direct repeat, reverse repeat, and Dyad repeat), and restriction sites that may interfere with cloning. A codon usage bias adjustment was performed using the distribution of codon usage frequency along the length of the gene sequence, with a resulting Codon Adaptation Index (CAI) of 0.95. A CAI of 1.0 is considered to be perfect in the desired expression organism, and a CAI of greater than 0.8 is regarded as good, in terms of high gene expression level. The Frequency of Optimal Codons (FOP) was measured as the percentage distribution of favorable codons in computed codon quality groups, with the value of 100 set for the codon with the highest usage frequency for a given amino acid in the desired expression organism. A result of 80% of the codons was found in the highest codon quality group of 91-100, 3% in the second highest quality group of 81-90, and 14% in the third highest quality group of 71-80. A GC content adjustment was performed resulting in an average GC content of 56.46%, with the ideal percentage range of GC content being between 30-70%. One optional HindIII cloning site (SEQ ID NO: 12) was incorporated at the 5-prime end at position 1 of the sequence; one optional Kpnl cloning site (SEQ ID NO: 13) was incorporated at the 3-prime end at position 2524 of the sequence.

The corresponding combined EFE and AKGP amino acid sequences expressed by SEQ ID. NO. 7 have a P2A cleavage sequence (SEQ ID NO: 11) inserted between the EFE and AKGP amino acid sequences. The encoded amino acid sequence having the EFE and AKGP sequence together is shown in SEQ ID NO. 5 (EFE-P2A_pSyn_6 (No His). An optional His-TEV sequence may be included at the N-terminus (SEQ ID NO: 10), resulting in the amino acid sequence of SEQ ID NO. 6 (EFE-P2A-aKGP_pSyn_6).

### Example 4: Lab Scale Experimental Procedures

1. Tailored-designed DNA constructions will be generated that encode the critical intermediates of a synthetic bio-ethylene pathway. 2. Carefully selected photosynthetic microorganisms will then be expanded for cloning and gene expression. 3. Genetic and metabolic engineering of microorganisms will then be performed for continuous production of bio-ethylene. 4. Bioengineered microorganisms will then be selected and expanded in a photobioreactor. 5. Bioreactor culture conditions (including CO₂ concentration, light exposure time and wave-length, temperature, pH) will be adapted. 6. Samples will be collected and analyzed by HPLC to measure bio-ethylene synthesis. 7. Bio-ethylene production in genetically engineered microorganisms will be adapted. 8. Ethylene production processes will be scaled up.

### Example 5: Engineering the Production of AKG in Cyanobacteria

Previous work has shown that deletion of the glgC gene leads to an increase in AKG production in Cyanobacteria. It has also been shown that over expression of the ppc gene (SEQ ID NO. 14, Genbank P74299), which encodes phosphoenolpyruvate synthase (SEQ ID NO. 15), and the gltA gene (SEQ ID NO. 16, Genbank Q59977), which encodes citrate synthase (SEQ ID NO. 17), can enhance the production of AKG as a substrate for producing other compounds. Based on this research, two categories of genes were chosen, including genes that are related directly to AKG synthesis and secretion pathways, including ppc and gltA (overexpression), and genes that are involved in energy storage pathways, including glgC (deletion), which plays a critical role in the glycogen synthesis pathway. A construct of ppc-p2A-gltA (SEQ ID NO. 18) was created for cloning into the pSyn6 plasmid before integration into Synechococcus elongatus and growth of transformed colonies. PCR was performed on pSyn6-PPC-gltA colonies to confirm the expression of the construct in Cyanobacteria; an expected band size for PPC-gltA of 4621 base pairs was observed.

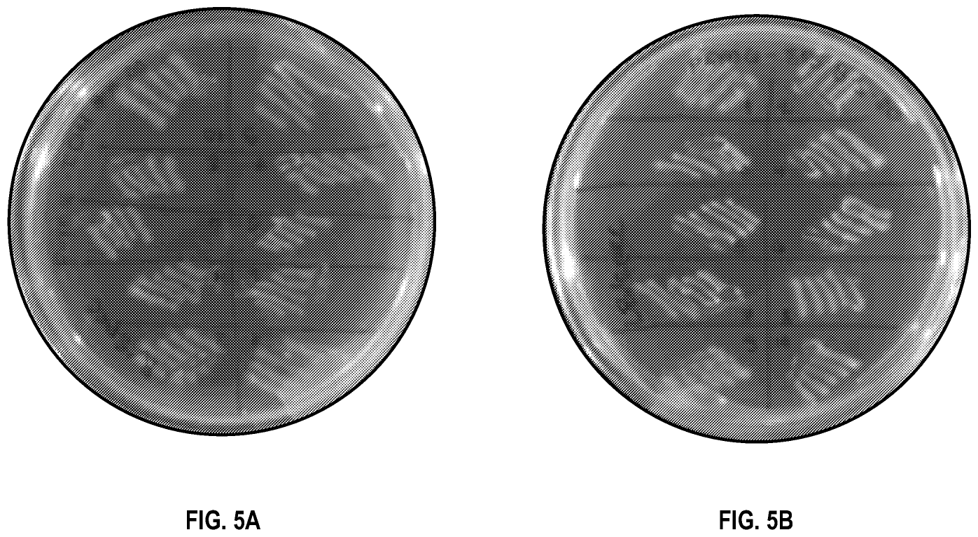

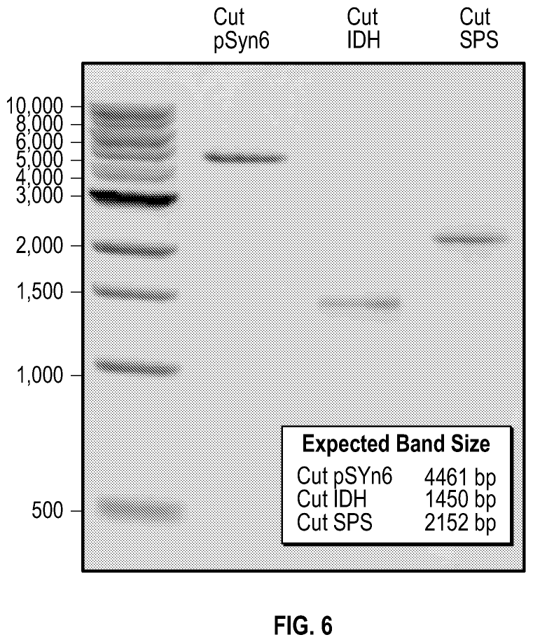

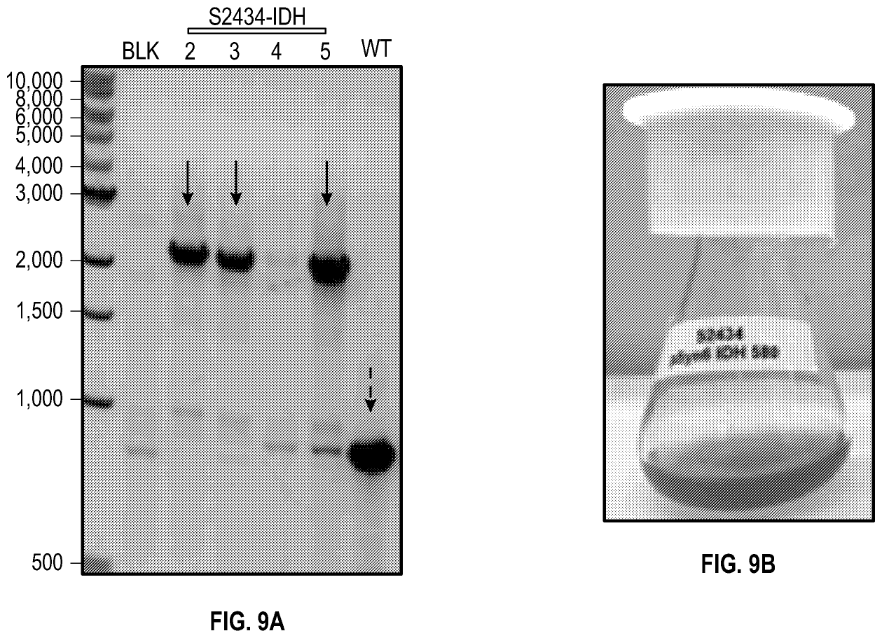

For the overexpression of AKG in Cyanobacteria, a construct of an IDH gene (SEQ ID NO: 19), which encodes isocitrate dehydrogenase (SEQ ID NO. 20), was made by cloning the IDH gene into the pSyn6 plasmid. Successful cloning of the IDH gene into the pSyn6-IDH plasmid was confirmed by growth of bacterial colonies FIG. 5A) and by gel electrophoresis and DNA analysis (FIG. 6). Synechococcus elongatus strain 52434-IDH integrating the IDH construct was confirmed by bacterial culture growth (FIG. 9B) and gel electrophoresis and DNA analysis (FIG. 9A). Cell culture growth was shown to be improved significantly by increasing the bicarbonate concentration in the growth medium by 0.5 g/L or 1.0 g/L. A plasmid for deletion of the glgC gene (SEQ ID NO. 21, Genbank CP000100.1), which encodes glucose-1-phosphate adenylyltransferase (SEQ ID NO. 22), in Cyanobacteria (Synechococcus elongatus), was also made and confirmed.

### Example 6: Engineering the Production of Sucrose in Cyanobacteria

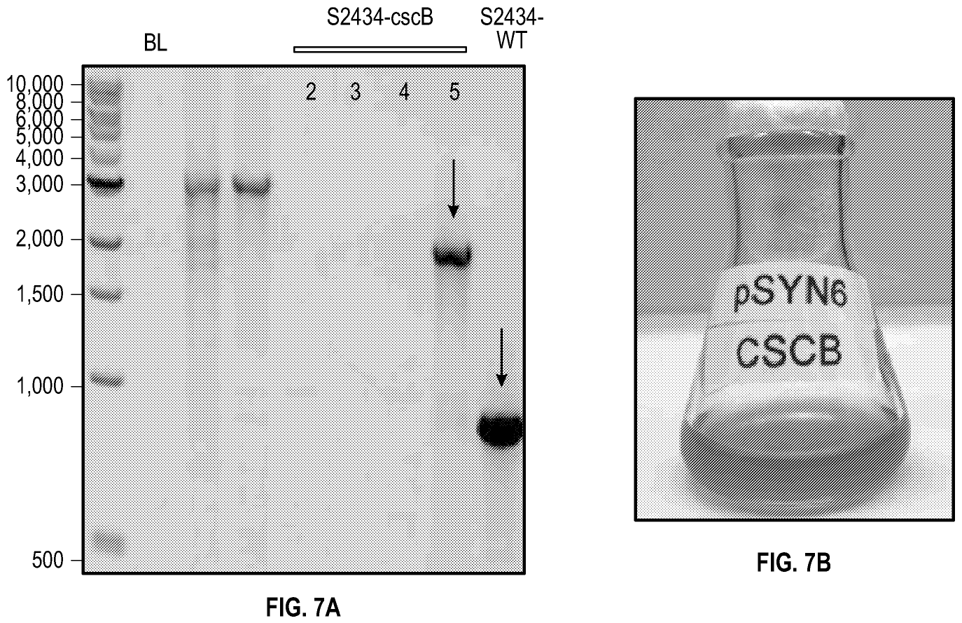

For production of sucrose in Cyanobacteria, a construct of a cscB gene from E. coli (SEQ ID. NO. 23, Genbank P300000), which encodes sucrose permease (SEQ ID NO. 24), was made by cloning of the cscB gene into the pSyn6 plasmid. Successful cloning of the cscB gene into the pSyn6-cscB construct was confirmed by bacterial colony growth (FIG. 5B) and by gel electrophoresis and DNA analysis (FIG. 6). Synechococcus elongatus strain UTEX 52434 (52434-cscB) integrating cscB was confirmed by bacterial culture growth (FIG. 7B) and by gel electrophoresis and DNA analysis (FIG. 7A).

In addition to the overexpression of the cscB gene, and the deletion of the glgC gene, other gene targets include overexpression of the sps gene (SEQ ID NO. 25, Genbank A0A0H3KOV9), which encodes sucrose phosphate synthase (SEQ ID NO. 26); the spp gene (SEQ ID NO. 27, Genbank Q7BII3), which encodes sucrose-6-phosphatase (SEQ ID NO. 28), the glgP gene (SEQ ID NO. 29, Genbank Q31RP3), which encodes glycogen phosphorylase (SEQ ID NO. 30), and the galU gene (SEQ ID NO. 31, Genbank P0AEP3), which encodes UTP-glucose-1-phosphate uridylyltransferase (SEQ ID NO. 32), to reroute the intermediates to sucrose. In addition, deletion of the inv gene (SEQ ID NO. 33, Genbank P74573), which encodes invertase (SEQ ID NO. 34), and the ggpS gene (SEQ ID NO. 35, Genbank P74258), which encodes glucosylglycerol-phosphate synthase (SEQ ID NO. 36), will prevent conversion to alternative products; and deletion of the glgA gene (SEQ ID NO. 37, Genbank P74521), which encodes glycogen synthase (SEQ ID NO. 38), will eliminate the conversion of substrate to glycogen, which potentially can increase the sucrose yield.

### Example 7: Engineering the Production of Ethylene in E. coli

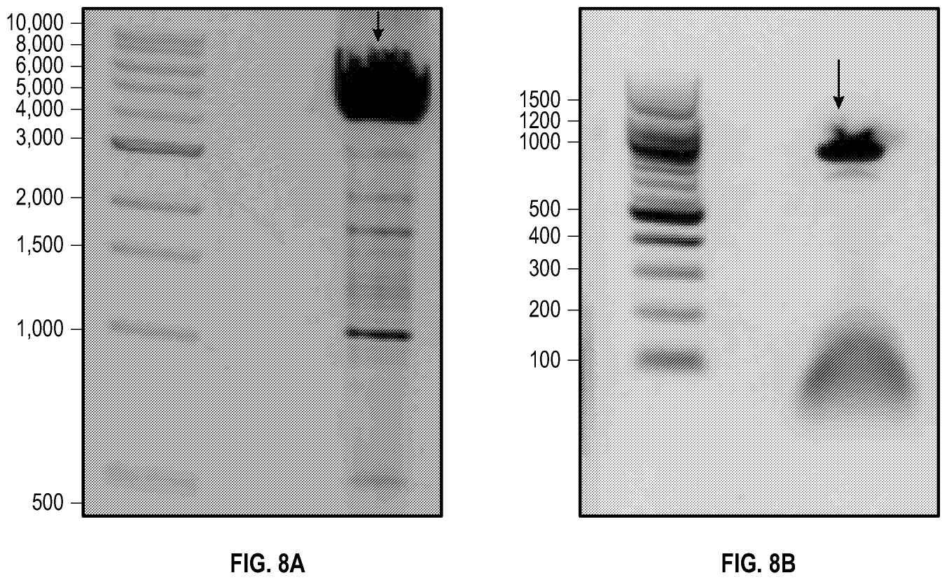

For engineering production of ethylene in E. coli, gene construct pUC-EFE (SEQ ID NO. 39) was made encoding ethylene forming enzyme (EFE) under an IPTG-inducible promoter in a high copy number plasmid, pUC19. Expression of the PUC-EFE plasmid in E. coli was confirmed by colony growth on agar media supplemented with ampicillin, IPTG and X-gal, and observance of the expected band size of 2322 base pairs by gel electrophoresis and DNA analysis (FIG. 8A and FIG. 8B). In FIG. 8A, the arrow shows the EFE DNA construct; in FIG. 8B, the arrow shows the DNA element controlling plasmid copy number. DNA sequencing results confirmed the presence of the plasmid. EFE production was confirmed by SDS-PAGE and Western blot analysis. Ethylene expression levels of 5 mg/L and 30% solubility were observed under induction conditions of 16 hours at 15 degrees Celsius.

For engineering production of ethylene in E. coli, a plasmid was constructed for continuous production of EFE in E. coli. In all approaches, the EFE expression was under control of the chloroplast psbA promoter. In a first construct EFE-AKGP-psbA (SEQ ID NO. 40), the EFE and AKGP genes were placed under control of the psbA promoter (SEQ ID NO. 41) and the rrnB terminator (SEQ ID NO. 42). In a second construct EFE-psbA (SEQ ID NO. 43), only EFE gene expression was placed under control of the psbA promoter (SEQ ID NO. 41) and the T7 terminator (SEQ ID NO. 44). Both constructs were cloned into a pUC19 plasmid backbone, to take advantage of the high copy number of the plasmid, before expressing the protein in E. coli BL21 (DE3), DH5alpha, or MG1655 cell lines. A pUC-psb-EFE plasmid was constructed (SEQ ID NO. 45).

The effect of growth media, as well as AKG and arginine supplementation, on ethylene production was measured. The results indicated that a maximum ethylene production of 0.037 lb/gallon/month for E. coli BL 21 PUC19 EFE was obtained when fermented under the conditions shown in Table 1.

TABLE 1
Conditions for the production of ethylene
for E. coli BL 21 PUC19 EFE at 30° C.

| Media | MOPS | |
|---|---|---|
| Glucose | 4 | g/L |
| IPTG | 0.5 | mM |
| Arginine | 3 | mM |
| AKG | 2 | mM |

| | |
|---|---|
| Induction | Induced at the start |

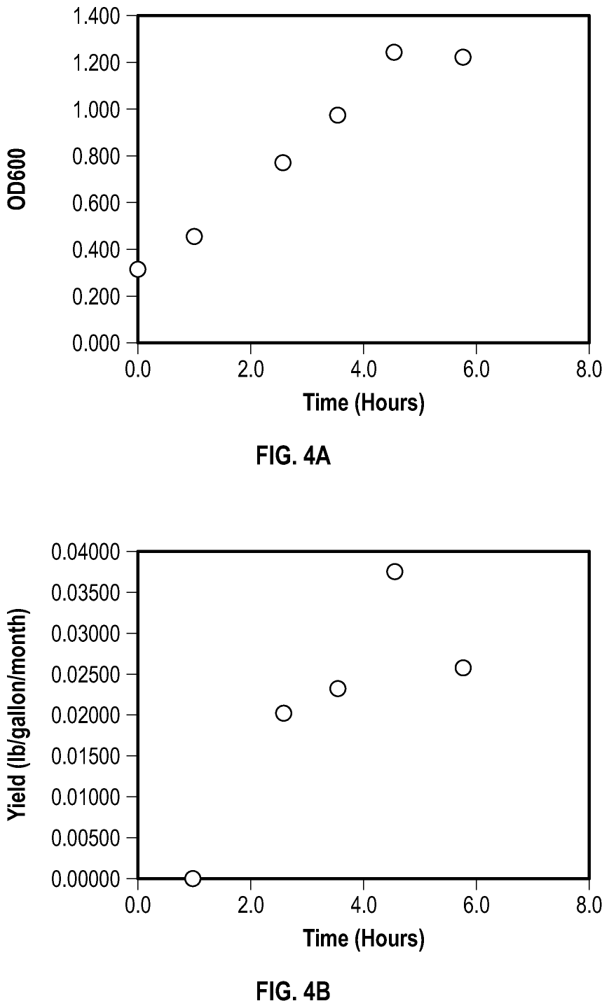

The results of the observed growth rate of E. coli BL 21 PUC19 EFE is shown in FIG. 4A. The observed ethylene yield under the conditions shown in Table 1 is shown in FIG. 4B. Gas chromatography analysis of headspace samples confirmed the production of ethylene by the E. coli culture.

APPENDIX

SEQ ID NO: 1 -
MIHAPSRWGVFPSLGLCSPDVVWNEHPSLYMDKEETSMTNLQTFELPTEVTGCAADI
SLGRALIQAWQKDGIFQIKTDSEQDRKTQEAMAASKQFCKEPLTFKSSCVSDLTYSG
YVASGEEVTAGKPDFPEIFTVCKDLSVGDQRVKAGWPCHGPVPWPNNTYQKSMKT
FMEELGLAGERLLKLTALGFELPINTFTDLTRDGWHHMRVLRFPPQTSTLSRGIGAHT
DYGLLVIAAQDDVGGLYIRPPVEGEKRNRNWLPGESSAGMFEHDEPWTFVTPTPGV
WTVFPGDILQFMTGGQLLSTPHKVKLNTRERFACAYFHEPNFEASAYPLFEPSANERI
HYGEHFTNMFMRCYPDRITTQRINKENRLAHLEDLKKYSDTRATGS
SEQ ID NO: 2-
MTESITSNGTLVASDTRRRVWAIVSASSGNLVEWFDFYVYSFCSLYFAHIFFPSGNTT
TQLLQTAGVFAAGFLMRPIGGWLFGRIADRRGRKTSMLISVCMMCFGSLIIACLPGY
DAIGTWAPALLLLARLFQGLSVGGEYGTSATYMSEIALEGRKGFYASFQYVTLIGGQ
LLAILVVVILQQILTDSQLHEWGWRIPFAMGAALAIVALWLRRQLDETSQKEVRALK
EAGSFKGLWRNRKAFLMVLGFTAGGSLSFYTFTTYMQKYLVNTTGMHANVASVIM
TAALFVFMLIQPLIGALSDKIGRRTSMLIFGGMSALCTVPILTALQHVSSPYAAFALV
MLAMVIVSFYTSISGILKAEMFPAQVRALGVGLSYAVANALFGGSAEYVALSLKSW
GSETTFFWYVTIMGALAFIVSLMLHRKGKGIRL
SEQ ID NO. 3-
ATGATTCACGCCCCGTCGCGCTGGGGCGTGTTTCCCTCGCTGGGCCTGTGCAGCC
CCGACGTGGTGTGGAACGAGCACCCGAGCCTGTACATGGACAAGGAGGAGACGT
CGATGACCAACCTGCAGACGTTCGAGCTGCCGACCGAGGTGACCGGCTGCGCCG
CCGACATCTCCCTGGGCCGGGCGCTGATCCAGGCGTGGCAGAAGGACGGCATCT
TCCAGATCAAGACCGACAGCGAGCAGGACCGGAAGACCCAGGAGGCGATGGCG
GCCTCCAAGCAGTTCTGCAAGGAGCCCCTGACCTTCAAGTCGTCCTGCGTCAGCG
ACCTGACCTACTCGGGCTACGTGGCCTCGGGCGAGGAGGTGACCGCCGGCAAGC
CGGACTTTCCGGAGATCTTCACCGTGTGCAAGGACCTGAGCGTGGGCGACCAGC
GGGTCAAGGCGGGCTGGCCCTGCCACGGCCCCGTGCCGTGGCCGAACAACACCT
ACCAGAAGTCCATGAAGACGTTCATGGAGGAGCTGGGCCTGGCCGGCGAGCGCC
TGCTGAAGCTGACCGCGCTGGGCTTCGAGCTGCCCATCAACACGTTCACCGACCT
GACCCGGGACGGCTGGCACCACATGCGCGTCCTGCGGTTTCCGCCCCAGACCAG
CACGCTGAGCCGCGGCATTGGCGCGCACACGGACTACGGCCTGCTGGTGATTGC
CGCGCAGGACGACGTGGGCGGCCTGTACATTCGCCCGCCGGTGGAGGGCGAGAA
GCGCAACCGGAACTGGCTGCCCGGCGAGTCCTCGGCGGGCATGTTCGAGCACGA
CGAGCCCTGGACGTTCGTGACCCCCACGCCGGGCGTGTGGACGGTGTTTCCCGGC
GACATCCTGCAGTTCATGACCGGCGGCCAGCTG
CTGTCGACGCCGCACAAGGTGAAGCTGAACACCCGGGAGCGCTTCGCCTGCGCG
TACTTCCACGAGCCGAACTTCGAGGCCTCGGCCTACCCCCTGTTCGAGCCCTCCG
CGAACGAGCGCATCCACTACGGCGAGCACTTCACCAATATGTTTATGCGCTGCTA
CCCCGACCGCATCACCACCCAGCGCATCAACAAGGAGAATCGCCTGGCGCACCT
GGAGGACCTGAAGAAGTACAGCGACACCCGCGCCACCGGCTCG
SEQ ID NO. 4-
ATGATACACGCTCCAAGTAGATGGGGAGTATTTCCCTCACTAGGGTTATGCAGCC
CGGACGTTGTGTGGAATGAGCATCCGAGCCTGTACATGGACAAAGAGGAAACCA
GCATGACCAACCTGCAGACCTTTGAACTGCCGACCGAAGTGACCGGTTGCGCGG
CGGACATCAGCCTGGGTCGTGCGCTGATTCAGGCGTGGCAAAAGGATGGTATCT
TCCAGATTAAAACCGACAGCGAGCAGGATCGTAAGACCCAAGAAGCGATGGCG
GCGAGCAAGCAATTTTGCAAAGAGCCGCTGACCTTCAAAAGCAGCTGCGTTAGC
GACCTGACCTACAGCGGTTATGTGGCGAGCGGCGAGGAAGTTACCGCGGGCAAG
CCGGATTTCCCGGAAATTTTTACCGTGTGCAAGGACCTGAGCGTGGGCGATCAGC
GTGTTAAAGCGGGTTGGCCGTGCCATGGTCCGGTTCCGTGGCCGAACAACACCTA
TCAAAAGAGCATGAAAACCTTTATGGAGGAACTGGGTCTGGCGGGCGAGCGTCT
GCTGAAACTGACCGCGCTGGGTTTTGAACTGCCGATCAACACCTTCACCGACCTG
ACCCGTGATGGCTGGCACCACATGCGTGTGCTGCGTTTCCCGCCGCAGACCAGCA
CCCTGAGCCGTGGTATTGGTGCGCACACCGACTACGGTCTGCTGGTGATTGCGGC
GCAAGACGATGTTGGTGGCCTGTATATCCGTCCGCCGGTGGAGGGCGAAAAGCG
TAACCGTAACTGGCTGCCGGGCGAGAGCAGCGCGGGCATGTTTGAGCACGACGA
ACCGTGGACCTTCGTTACCCCGACCCCGGGTGTGTGGACCGTTTTTCCGGGCGAT
ATTCTGCAGTTCATGACCGGTGGCCAACTGCTGAGCACCCCGCACAAGGTTAAAC
TGAACACCCGTGAACGTTTCGCGTGCGCGTACTTTCACGAGCCGAACTTCGAAGC
GAGCGCGTATCCGCTGTTCGAGCCGAGCGCGAACGAACGTATCCACTACGGCGA
GCACTTCACCAACATGTTTATGCGTTGCTATCCGGATCGTATCACCACCCAACGT
ATTAACAAAGAAAACCGTCTGGCGCACCTGGAAGACCTGAAGAAATACAGCGAC
ACCCGTGCGACCGGCAGC
SEQ ID NO. 5-
MIHAPSRWGVFPSLGLCSPDVVWNEHPSLYMDKEETSMTNLQTFELPTEVTGCAADI
SLGRALIQAWQKDGIFQIKTDSEQDRKTQEAMAASKQFCKEPLTFKSSCVSDLTYSG
YVASGEEVTAGKPDFPEIFTVCKDLSVGDQRVKAGWPCHGPVPWPNNTYQKSMKT
FMEELGLAGERLLKLTALGFELPINTFTDLTRDGWHHMRVLRFPPQTSTLSRGIGAHT
DYGLLVIAAQDDVGGLYIRPPVEGEKRNRNWLPGESSAGMFEHDEPWTFVTPTPGV
WTVFPGDILQFMTGGQLLSTPHKVKLNTRERFACAYFHEPNFEASAYPLFEPSANERI
HYGEHFTNMFMRCYPDRITTQRINKENRLAHLEDLKKYSDTRATGSGATNFSLLKQ
AGDVEENPGPMTESITSNGTLVASDTRRRVWAIVSASSGNLVEWFDFYVYSFCSLYF
AHIFFPSGNTTTQLLQTAGVFAAGFLMRPIGGWLFGRIADRRGRKTSMLISVCMMCF
GSLIIACLPGYDAIGTWAPALLLLARLFQGLSVGGEYGTSATYMSEIALEGRKGFYAS
FQYVTLIGGQLLAILVVVILQQILTDSQLHEWGWRIPFAMGAALAIVALWLRRQLDE
TSQKEVRALKEAGSFKGLWRNRKAFLMVLGFTAGGSLSFYTFTTYMQKYLVNTTG
MHANVASVIMTAALFVFMLIQPLIGALSDKIGRRTSMLIFGGMSALCTVPILTALQHV
SSPYAAFALVMLAMVIVSFYTSISGILKAEMFPAQVRALGVGLSYAVANALFGGSAE
SEQ ID. NO. 6-
MHHHHHHENLYFQGKLMIHAPSRWGVFPSLGLCSPDVVWNEHPSLYMDKEETSMT
NLQTFELPTEVTGCAADISLGRALIQAWQKDGIFQIKTDSEQDRKTQEAMAASKQFC
KEPLTFKSSCVSDLTYSGYVASGEEVTAGKPDFPEIFTVCKDLSVGDQRVKAGWPCH
GPVPWPNNTYQKSMKTFMEELGLAGERLLKLTALGFELPINTFTDLTRDGWHHMRV
LRFPPQTSTLSRGIGAHTDYGLLVIAAQDDVGGLYIRPPVEGEKRNRNWLPGESSAG
MFEHDEPWTFVTPTPGVWTVFPGDILQFMTGGQLLSTPHKVKLNTRERFACAYFHEP
NFEASAYPLFEPSANERIHYGEHFTNMFMRCYPDRITTQRINKENRLAHLEDLKKYS
DTRATGSGATNFSLLKQAGDVEENPGPMTESITSNGTLVASDTRRRVWAIVSASSGN
LVEWFDFYVYSFCSLYFAHIFFPSGNTTTQLLQTAGVFAAGFLMRPIGGWLFGRIADR
RGRKTSMLISVCMMCFGSLIIACLPGYDAIGTWAPALLLLARLFQGLSVGGEYGTSA
TYMSEIALEGRKGFYASFQYVTLIGGQLLAILVVVILQQILTDSQLHEWGWRIPFAMG
AALAIVALWLRRQLDETSQKEVRALKEAGSFKGLWRNRKAFLMVLGFTAGGSLSFY
TFTTYMQKYLVNTTGMHANVASVIMTAALFVFMLIQPLIGALSDKIGRRTSMLIFGG
MSALCTVPILTALQHVSSPYAAFALVMLAMVIVSFYTSISGILKAEMFPAQVRALGV
GLSYAVANALFGGSAEYVALSLKSWGSETTFFWYVTIMGALAFIVSLMLHRKGKGI
RL
SEQ ID NO. 7-
ATGATTCATGCCCCCTCCCGCTGGGGCGTGTTTCCCAGTCTGGGTCTCTGCTCCCC
CGATGTGGTGTGGAACGAACACCCCAGCCTGTACATGGATAAGGAAGAGACCAG
TATGACCAATCTGCAAACCTTTGAACTGCCCACCGAGGTGACCGGTTGCGCCGCC
GATATTAGCCTCGGTCGCGCCCTGATTCAAGCCTGGCAAAAGGATGGCATCTTCC
AAATCAAGACCGATTCCGAACAAGATCGCAAGACCCAAGAGGCCATGGCCGCCA
GCAAACAATTTTGCAAGGAACCCCTGACCTTTAAATCCAGCTGCGTGAGCGATCT
CACCTACAGTGGCTATGTGGCCAGTGGTGAAGAGGTGACCGCCGGCAAGCCCGA
TTTTCCCGAGATTTTTACCGTGTGCAAGGATCTGAGTGTGGGTGATCAACGCGTG
AAAGCCGGTTGGCCCTGCCATGGTCCCGTGCCCTGGCCCAACAATACCTATCAAA
AATCCATGAAGACCTTTATGGAAGAACTCGGTCTGGCCGGTGAACGCCTGCTCA
AACTGACCGCCCTCGGCTTTGAGCTGCCCATTAACACCTTTACCGATCTCACCCG
CGATGGTTGGCACCACATGCGCGTGCTGCGCTTTCCTCCCCAAACCAGCACCCTG
AGCCGCGGTATTGGTGCCCACACCGATTACGGCCTGCTCGTGATTGCCGCCCAAG
ATGATGTGGGCGGTCTGTATATTCGCCCTCCCGTGGAAGGCGAGAAACGCAACC
GCAATTGGCTCCCCGGCGAAAGTTCCGCCGGCATGTTTGAACACGATGAACCCTG
GACCTTTGTGACGCCCACGCCCGGCGTGTGGACCGTGTTTCCCGGTGATATTCTG
CAATTTATGACCGGCGGTCAACTGCTCTCCACGCCCCACAAAGTGAAGCTCAACA
CCCGCGAACGCTTTGCCTGCGCCTACTTTCACGAACCCAATTTTGAGGCCAGTGC
CTATCCCCTGTTTGAACCCTCCGCCAACGAGCGCATTCACTACGGCGAGCACTTT
ACCAATATGTTTATGCGCTGCTATCCCGATCGCATTACCACCCAACGCATTAACA
AGGAAAATCGCCTGGCCCACCTCGAGGATCTGAAAAAGTATAGTGATACCCGCG
CCACCGGTAGTGGTGCCACCAACTTTAGCCTGCTCAAACAAGCCGGCGATGTGG
AAGAGAACCCCGGTCCCATGACCGAAAGTATTACCAGCAATGGCACCCTGGTGG
CCAGTGATACCCGTCGCCGCGTGTGGGCCATTGTGAGTGCCAGCAGTGGTAACCT
GGTGGAGTGGTTTGATTTTTACGTGTATAGCTTTTGCAGTCTCTACTTTGCCCACA
ttttctttcccagtggcaataccaccacccaactgctgcaaaccgccggcgtgtt
TGCCGCCGGTTTTCTGATGCGCCCCATTGGCGGTTGGCTCTTTGGCCGCATTGCCG
ATCGTCGCGGTCGCAAGACCAGCATGCTGATTAGCGTGTGCATGATGTGCTTTGG
CTCCCTGATTATTGCCTGCCTCCCCGGCTATGATGCCATTGGCACCTGGGCCCCC
GCCCTGCTCCTGCTGGCCCGCCTCTTTCAAGGCCTGAGCGTGGGCGGTGAATACG
GCACCAGCGCCACCTATATGAGTGAAATTGCCCTGGAGGGCCGCAAAGGTTTTT
ACGCCAGTTTTCAATATGTGACCCTGATTGGCGGTCAACTGCTCGCCATTCTCGT
GGTGGTGATTCTCCAACAAATTCTGACCGATTCCCAACTGCACGAATGGGGCTGG
CGCATTCCCTTTGCCATGGGTGCCGCCCTGGCCATTGTGGCCCTGTGGCTCCGTC
GCCAACTCGATGAAACCAGCCAAAAAGAGGTGCGCGCCCTGAAAGAAGCCGGC
AGTTTTAAAGGTCTCTGGCGCAACCGCAAGGCCTTTCTCATGGTGCTGGGCTTTA
CCGCCGGCGGTAGTCTGTCCTTTTACACCTTTACCACCTACATGCAAAAATATCT
CGTGAACACCACCGGCATGCACGCCAATGTGGCCAGCGTGATTATGACCGCCGC
CCTGTTTGTGTTTATGCTCATTCAACCCCTGATTGGCGCCCTCAGCGATAAGATTG
GTCGTCGCACCAGTATGCTGATTTTTGGCGGTATGAGTGCCCTCTGCACCGTGCC
CATTCTCACCGCCCTGCAACACGTGTCCAGCCCCTACGCCGCCTTTGCCCTCGTG
ATGCTGGCCATGGTGATTGTGTCCTTTTATACCAGCATTAGTGGCATTCTGAAGG
CCGAAATGTTTCCCGCCCAAGTGCGCGCCCTGGGCGTGGGTCTCAGTTACGCCGT
GGCCAATGCCCTGTTTGGCGGTTCCGCCGAATATGTGGCCCTGTCCCTCAAAAGC
TGGGGCAGTGAGACCACCTTTTTCTGGTACGTGACCATTATGGGTGCCCTGGCCT
TTATTGTGAGCCTGATGCTCCACCGCAAAGGCAAGGGTATTCGCCTCTAG
SEQ ID NO. 8:-CATATG
SEQ ID NO. 9:-CACCACCACCATCATCATTAATGAAAGCTT
SEQ ID NO. 10:-MHHHHHHENLYFQGKL
SEQ ID NO. 11:-GATNFSLLKQAGDVEENPGP
SEQ ID NO. 12:-AAGCTT
SEQ ID NO. 13:-GGTACC
SEQ ID NO. 14:-
ATGACTGATTTTTTACGCGATGACATCAGGTTCCTCGGTCAAATCCTCGGTGAGG
TAATTGCGGAACAAGAAGGCCAGGAGGTTTATGAACTGGTCGAACAAGCGCGCC
TGACTTCTTTTGATATCGCCAAGGGCAACGCCGAAATGGATAGCCTGGTTCAGGT
TTTCGACGGCATTACTCCAGCCAAGGCAACACCGATTGCTCGCGCATTTTCCCAC
TTCGCTCTGCTGGCTAACCTGGCGGAAGACCTCTACGATGAAGAGCTTCGTGAAC
AGGCTCTCGATGCAGGCGACACCCCTCCGGACAGCACTCTTGATGCCACCTGGCT
GAAACTCAATGAGGGCAATGTTGGCGCAGAAGCTGTGGCCGATGTGCTGCGCAA
TGCTGAGGTGGCGCCGGTTCTGACTGCGCACCCAACTGAGACTCGCCGCCGCACT
GTTTTTGATGCGCAAAAGTGGATCACCACCCACATGCGTGAACGCCACGCTTTGC
AGTCTGCGGAGCCTACCGCTCGTACGCAAAGCAAGTTGGATGAGATCGAGAAGA
ACATCCGCCGTCGCATCACCATTTTGTGGCAGACCGCGTTGATTCGTGTGGCCCG
CCCACGTATCGAGGACGAGATCGAAGTAGGGCTGCGCTACTACAAGCTGAGCCT
TTTGGAAGAGATTCCACGTATCAACCGTGATGTGGCTGTTGAGCTTCGTGAGCGT
TTCGGCGAGGGTGTTCCTTTGAAGCCCGTGGTCAAGCCAGGTTCCTGGATTGGTG
GAGACCACGACGGTAACCCTTATGTCACCGCGGAAACAGTTGAGTATTCCACTC
ACCGCGCTGCGGAAACCGTGCTCAAGTACTATGCACGCCAGCTGCATTCCCTCGA
GCATGAGCTCAGCCTGTCGGACCGCATGAATAAGGTCACCCCGCAGCTGCTTGC
GCTGGCAGATGCAGGGCACAACGACGTGCCAAGCCGCGTGGATGAGCCTTATCG
ACGCGCCGTCCATGGCGTTCGCGGACGTATCCTCGCGACGACGGCCGAGCTGAT
CGGCGAGGACGCCGTTGAGGGCGTGTGGTTCAAGGTCTTTACTCCATACGCATCT
CCGGAAGAATTCTTAAACGATGCGTTGACCATTGATCATTCTCTGCGTGAATCCA
AGGACGTTCTCATTGCCGATGATCGTTTGTCTGTGCTGATTTCTGCCATCGAGAG
CTTTGGATTCAACCTTTACGCACTGGATCTGCGCCAAAACTCCGAAAGCTACGAG
GACGTCCTCACCGAGCTTTTCGAACGCGCCCAAGTCACCGCAAACTACCGCGAG
CTGTCTGAAGCAGAGAAACTTGAGGTGCTGCTGAAGGAACTGCGCAGCCCTCGT
CCGCTGATCCCGCACGGTTCAGATGAATACAGCGAGGTCACCGACCGCGAGCTC
GGCATCTTCCGCACCGCGTCGGAGGCTGTTAAGAAATTCGGGCCACGGATGGTG
CCTCACTGCATCATCTCCATGGCATCATCGGTCACCGATGTGCTCGAGCCGATGG
TGTTGCTCAAGGAATTCGGACTCATCGCAGCCAACGGCGACAACCCACGCGGCA
CCGTCGATGTCATCCCACTGTTCGAAACCATCGAAGATCTCCAGGCCGGCGCCGG
AATCCTCGACGAACTGTGGAAAATTGATCTCTACCGCAACTACCTCCTGCAGCGC
GACAACGTCCAGGAAGTCATGCTCGGTTACTCCGATTCCAACAAGGATGGCGGA
TATTTCTCCGCAAACTGGGCGCTTTACGACGCGGAACTGCAGCTCGTCGAACTAT
GCCGATCAGCCGGGGTCAACGTTCGCCTGTTCCACGGCCGTGGTGGCACCGTCGG
CCGCGGTGGCGGACCTTCCTACGACGCGATTCTTGCCCAGCCCAGGGGGGCTGTC
CAAGGTTCCGTGCGCATCACCGAGCAGGGCGAGATCATCTCCGCTAAGTACGGC
AACCCCGAAACCGCGCGCCGAAACCTCGAAGCCCTGGTCTCAGCCACGCTTGAG
GCATCGCTTCTCGACGTCTCCGAACTCACCGATCACCAACGCGCGTACGACATCA
TGAGTGAGATCTCTGAGCTCAGCTTGAAGAAGTACGCCTCCTTGGTGCACGAGG
ATCAAGGCTTCATCGATTACTTCACCCAGTCCACGCCGCTGCAGGAGATTGGATC
CCTCAACATCGGATCCAGGCCTTCCTCACGCAAGCAGACCTCCTCGGTGGAAGAT
TTGCGAGCCATCCCATGGGTGCTCAGCTGGTCACAGTCTCGTGTCATGCTGCCAG
GCTGGTTTGGTGTCGGAACCGCATTAGAGCAGTGGATTGGCGAAGGGGAGCAGG
CCACCCAACGCATTGCCGAGCTGCAAACACTCAATGAGTCCTGGCCATTTTTACC
CTCAGTGTTGGATAACATGGCTCAGGTGATGTCCAAGGCAGAGCTGCGTTTGGCA
AAGCTCTACGCAGACCTGATCCCAGATACGGAAGTAGCCGAGCGAGTCTATTCC
GTCATCCGCGAGGAGTACTTCCTGACCAAGAAGATGTTCTGCGTAATCACCGGCT
CTGATGATCTGCTTGATGACAACCCACTTCTCGCACGCTCTGTCCAGCGCCGATA
CCCCTACCTGCTTCCACTCAACGTGATCCAGGTAGAGATGATGCGACGCTACCGA
AAAGGCGACCAAAGCGAGCAAGTGTCCCGCAACATTCAGCTGACCATGAACGGT
CTTTCCACTGCGGTGCGCAACTCCGGC
SEQ ID NO. 15-
MTDFLRDDIRFLGQILGEVIAEQEGQEVYELVEQARLTSFDIAKGNAEMDSLVQVFD
GITPAKATPIARAFSHFALLANLAEDLYDEELREQALDAGDTPPDSTLDATWLKLNE
GNVGAEAVADVLRNAEVAPVLTAHPTETRRRTVFDAQKWITTHMRERHALQSAEP
TARTQSKLDEIEKNIRRRITILWQTALIRVARPRIEDEIEVGLRYYKLSLLEEIPRINRDV
AVELRERFGEGVPLKPVVKPGSWIGGDHDGNPYVTAETVEYSTHRAAETVLKYYAR
QLHSLEHELSLSDRMNKVTPQLLALADAGHNDVPSRVDEPYRRAVHGVRGRILATT
AELIGEDAVEGVWFKVFTPYASPEEFLNDALTIDHSLRESKDVLIADDRLSVLISAIES
FGFNLYALDLRQNSESYEDVLTELFERAQVTANYRELSEAEKLEVLLKELRSPRPLIP
HGSDEYSEVTDRELGIFRTASEAVKKFGPRMVPHCIISMASSVTDVLEPMVLLKEFGL
IAANGDNPRGTVDVIPLFETIEDLQAGAGILDELWKIDLYRNYLLQRDNVQEVMLGY
SDSNKDGGYFSANWALYDAELQLVELCRSAGVNVRLFHGRGGTVGRGGGPSYDAI
LAQPRGAVQGSVRITEQGEIISAKYGNPETARRNLEALVSATLEASLLDVSELTDHQR
AYDIMSEISELSLKKYASLVHEDQGFIDYFTQSTPLQEIGSLNIGSRPSSRKQTSSVEDL
RAIPWVLSWSQSRVMLPGWFGVGTALEQWIGEGEQATQRIAELQTLNESWPFLPSV
LDNMAQVMSKAELRLAKLYADLIPDTEVAERVYSVIREEYFLTKKMFCVITGSDDLL
DDNPLLARSVQRRYPYLLPLNVIQVEMMRRYRKGDQSEQVSRNIQLTMNGLSTAVR
NSG
SEQ ID NO. 16-
ATGTTTGAAAGGGATATCGTGGCTACTGATAACAACAAGGCTGTCCTGCACTACC
CCGGTGGCGAGTTCGAAATGGACATCATCGAGGCTTCTGAGGGTAACAACGGTG
TTGTCCTGGGCAAGATGCTGTCTGAGACTGGACTGATCACTTTTGACCCAGGTTA
TGTGAGCACTGGCTCCACCGAGTCGAAGATCACCTACATCGATGGCGATGCGGG
AATCCTGCGTTACCGCGGCTATGACATCGCTGATCTGGCTGAGAATGCCACCTTC
AACGAGGTTTCTTACCTACTTATCAACGGTGAGCTACCAACCCCAGATGAGCTTC
ACAAGTTTAACGACGAGATTCGCCACCACACCCTTCTGGACGAGGACTTCAAGTC
CCAGTTCAACGTGTTCCCACGCGACGCTCACCCAATGGCAACCTTGGCTTCCTCG
GTTAACATTTTGTCTACCTACTACCAGGACCAGCTGAACCCACTCGATGAGGCAC
AGCTTGATAAGGCAACCGTTCGCCTCATGGCAAAGGTTCCAATGCTGGCTGCGTA
CGCACACCGCGCACGCAAGGGTGCTCCTTACATGTACCCAGACAACTCCCTCAAT
GCGCGTGAGAACTTCCTGCGCATGATGTTCGGTTACCCAACCGAGCCATACGAG
ATCGACCCAATCATGGTCAAGGCTCTGGACAAGCTGCTCATCCTGCACGCTGACC
ACGAGCAGAACTGCTCCACCTCCACCGTTCGTATGATCGGTTCCGCACAGGCCAA
CATGTTTGTCTCCATCGCTGGTGGCATCAACGCTCTGTCCGGCCCACTGCACGGT
GGCGCAAACCAGGCTGTTCTGGAGATGCTCGAAGACATCAAGAGCAACCACGGT
GGCGACGCAACCGAGTTCATGAACAAGGTCAAGAACAAGGAAGACGGCGTCCG
CCTCATGGGCTTCGGACACCGCGTTTACAAGAACTACGATCCACGTGCAGCAATC
GTCAAGGAGACCGCACACGAGATCCTCGAGCACCTCGGTGGCGACGATCTTCTG
GATCTGGCAATCAAGCTGGAAGAAATTGCACTGGCTGATGATTACTTCATCTCCC
GCAAGCTCTACCCGAACGTAGACTTCTACACCGGCCTGATCTACCGCGCAATGGG
CTTCCCAACTGACTTCTTCACCGTATTGTTCGCAATCGGTCGTCTGCCAGGATGG
ATCGCTCACTACCGCGAGCAGCTCGGTGCAGCAGGCAACAAGATCAACCGCCCA
CGCCAGGTCTACACCGGCAACGAATCCCGCAAGTTGGTTCCTCGCGAGGAGCGC
TAA
SEQ ID NO. 17-
MFERDIVATDNNKAVLHYPGGEFEMDIIEASEGNNGVVLGKMLSETGLITFDPGYVS
TGSTESKITYIDGDAGILRYRGYDIADLAENATFNEVSYLLINGELPTPDELHKFNDEI
RHHTLLDEDFKSQFNVFPRDAHPMATLASSVNILSTYYQDQLNPLDEAQLDKATVRL
MAKVPMLAAYAHRARKGAPYMYPDNSLNARENFLRMMFGYPTEPYEIDPIMVKAL
DKLLILHADHEQNCSTSTVRMIGSAQANMFVSIAGGINALSGPLHGGANQAVLEMLE
DIKSNHGGDATEFMNKVKNKEDGVRLMGFGHRVYKNYDPRAAIVKETAHEILEHL
GGDDLLDLAIKLEEIALADDYFISRKLYPNVDFYTGLIYRAMGFPTDFFTVLFAIGRLP
GWIAHYREQLGAAGNKINRPRQVYTGNESRKLVPREER
SEQ ID NO. 18-
ATGACTGATTTTTTACGCGATGACATCAGGTTCCTCGGTCAAATCCTCGGTGAGG
TAATTGCGGAACAAGAAGGCCAGGAGGTTTATGAACTGGTCGAACAAGCGCGCC
TGACTTCTTTTGATATCGCCAAGGGCAACGCCGAAATGGATAGCCTGGTTCAGGT
TTTCGACGGCATTACTCCAGCCAAGGCAACACCGATTGCTCGCGCATTTTCCCAC
TTCGCTCTGCTGGCTAACCTGGCGGAAGACCTCTACGATGAAGAGCTTCGTGAAC
AGGCTCTCGATGCAGGCGACACCCCTCCGGACAGCACTCTTGATGCCACCTGGCT
GAAACTCAATGAGGGCAATGTTGGCGCAGAAGCTGTGGCCGATGTGCTGCGCAA
TGCTGAGGTGGCGCCGGTTCTGACTGCGCACCCAACTGAGACTCGCCGCCGCACT
GTTTTTGATGCGCAAAAGTGGATCACCACCCACATGCGTGAACGCCACGCTTTGC
AGTCTGCGGAGCCTACCGCTCGTACGCAAAGCAAGTTGGATGAGATCGAGAAGA
ACATCCGCCGTCGCATCACCATTTTGTGGCAGACCGCGTTGATTCGTGTGGCCCG
CCCACGTATCGAGGACGAGATCGAAGTAGGGCTGCGCTACTACAAGCTGAGCCT
TTTGGAAGAGATTCCACGTATCAACCGTGATGTGGCTGTTGAGCTTCGTGAGCGT
TTCGGCGAGGGTGTTCCTTTGAAGCCCGTGGTCAAGCCAGGTTCCTGGATTGGTG
GAGACCACGACGGTAACCCTTATGTCACCGCGGAAACAGTTGAGTATTCCACTC
ACCGCGCTGCGGAAACCGTGCTCAAGTACTATGCACGCCAGCTGCATTCCCTCGA
GCATGAGCTCAGCCTGTCGGACCGCATGAATAAGGTCACCCCGCAGCTGCTTGC
GCTGGCAGATGCAGGGCACAACGACGTGCCAAGCCGCGTGGATGAGCCTTATCG
ACGCGCCGTCCATGGCGTTCGCGGACGTATCCTCGCGACGACGGCCGAGCTGAT
CGGCGAGGACGCCGTTGAGGGCGTGTGGTTCAAGGTCTTTACTCCATACGCATCT
CCGGAAGAATTCTTAAACGATGCGTTGACCATTGATCATTCTCTGCGTGAATCCA
AGGACGTTCTCATTGCCGATGATCGTTTGTCTGTGCTGATTTCTGCCATCGAGAG
CTTTGGATTCAACCTTTACGCACTGGATCTGCGCCAAAACTCCGAAAGCTACGAG
GACGTCCTCACCGAGCTTTTCGAACGCGCCCAAGTCACCGCAAACTACCGCGAG
CTGTCTGAAGCAGAGAAACTTGAGGTGCTGCTGAAGGAACTGCGCAGCCCTCGT
CCGCTGATCCCGCACGGTTCAGATGAATACAGCGAGGTCACCGACCGCGAGCTC
GGCATCTTCCGCACCGCGTCGGAGGCTGTTAAGAAATTCGGGCCACGGATGGTG
CCTCACTGCATCATCTCCATGGCATCATCGGTCACCGATGTGCTCGAGCCGATGG
TGTTGCTCAAGGAATTCGGACTCATCGCAGCCAACGGCGACAACCCACGCGGCA
CCGTCGATGTCATCCCACTGTTCGAAACCATCGAAGATCTCCAGGCCGGCGCCGG
AATCCTCGACGAACTGTGGAAAATTGATCTCTACCGCAACTACCTCCTGCAGCGC
GACAACGTCCAGGAAGTCATGCTCGGTTACTCCGATTCCAACAAGGATGGCGGA
TATTTCTCCGCAAACTGGGCGCTTTACGACGCGGAACTGCAGCTCGTCGAACTAT
GCCGATCAGCCGGGGTCAACGTTCGCCTGTTCCACGGCCGTGGTGGCACCGTCGG
CCGCGGTGGCGGACCTTCCTACGACGCGATTCTTGCCCAGCCCAGGGGGGCTGTC
CAAGGTTCCGTGCGCATCACCGAGCAGGGCGAGATCATCTCCGCTAAGTACGGC
AACCCCGAAACCGCGCGCCGAAACCTCGAAGCCCTGGTCTCAGCCACGCTTGAG
GCATCGCTTCTCGACGTCTCCGAACTCACCGATCACCAACGCGCGTACGACATCA
TGAGTGAGATCTCTGAGCTCAGCTTGAAGAAGTACGCCTCCTTGGTGCACGAGG
ATCAAGGCTTCATCGATTACTTCACCCAGTCCACGCCGCTGCAGGAGATTGGATC
CCTCAACATCGGATCCAGGCCTTCCTCACGCAAGCAGACCTCCTCGGTGGAAGAT
TTGCGAGCCATCCCATGGGTGCTCAGCTGGTCACAGTCTCGTGTCATGCTGCCAG
GCTGGTTTGGTGTCGGAACCGCATTAGAGCAGTGGATTGGCGAAGGGGAGCAGG
CCACCCAACGCATTGCCGAGCTGCAAACACTCAATGAGTCCTGGCCATTTTTACC
CTCAGTGTTGGATAACATGGCTCAGGTGATGTCCAAGGCAGAGCTGCGTTTGGCA
AAGCTCTACGCAGACCTGATCCCAGATACGGAAGTAGCCGAGCGAGTCTATTCC
GTCATCCGCGAGGAGTACTTCCTGACCAAGAAGATGTTCTGCGTAATCACCGGCT
CTGATGATCTGCTTGATGACAACCCACTTCTCGCACGCTCTGTCCAGCGCCGATA
CCCCTACCTGCTTCCACTCAACGTGATCCAGGTAGAGATGATGCGACGCTACCGA
AAAGGCGACCAAAGCGAGCAAGTGTCCCGCAACATTCAGCTGACCATGAACGGT
CTTTCCACTGCGGTGCGCAACTCCGGCGCCACCAACTCAACTCCGGCGCCACCAA
CTTTAGCCTGCTCAAACAAGCCGGCGATGTGGAAGAGAACCCCGGTCCCATGTTT
GAAAGGGATATCGTGGCTACTGATAACAACAAGGCTGTCCTGCACTACCCCGGT
GGCGAGTTCGAAATGGACATCATCGAGGCTTCTGAGGGTAACAACGGTGTTGTC
CTGGGCAAGATGCTGTCTGAGACTGGACTGATCACTTTTGACCCAGGTTATGTGA
GCACTGGCTCCACCGAGTCGAAGATCACCTACATCGATGGCGATGCGGGAATCC
TGCGTTACCGCGGCTATGACATCGCTGATCTGGCTGAGAATGCCACCTTCAACGA
GGTTTCTTACCTACTTATCAACGGTGAGCTACCAACCCCAGATGAGCTTCACAAG
TTTAACGACGAGATTCGCCACCACACCCTTCTGGACGAGGACTTCAAGTCCCAGT
TCAACGTGTTCCCACGCGACGCTCACCCAATGGCAACCTTGGCTTCCTCGGTTAA
CATTTTGTCTACCTACTACCAGGACCAGCTGAACCCACTCGATGAGGCACAGCTT
GATAAGGCAACCGTTCGCCTCATGGCAAAGGTTCCAATGCTGGCTGCGTACGCA
CACCGCGCACGCAAGGGTGCTCCTTACATGTACCCAGACAACTCCCTCAATGCGC
GTGAGAACTTCCTGCGCATGATGTTCGGTTACCCAACCGAGCCATACGAGATCGA
CCCAATCATGGTCAAGGCTCTGGACAAGCTGCTCATCCTGCACGCTGACCACGAG
CAGAACTGCTCCACCTCCACCGTTCGTATGATCGGTTCCGCACAGGCCAACATGT
TTGTCTCCATCGCTGGTGGCATCAACGCTCTGTCCGGCCCACTGCACGGTGGCGC
AAACCAGGCTGTTCTGGAGATGCTCGAAGACATCAAGAGCAACCACGGTGGCGA
CGCAACCGAGTTCATGAACAAGGTCAAGAACAAGGAAGACGGCGTCCGCCTCAT
GGGCTTCGGACACCGCGTTTACAAGAACTACGATCCACGTGCAGCAATCGTCAA
GGAGACCGCACACGAGATCCTCGAGCACCTCGGTGGCGACGATCTTCTGGATCT
GGCAATCAAGCTGGAAGAAATTGCACTGGCTGATGATTACTTCATCTCCCGCAAG
CTCTACCCGAACGTAGACTTCTACACCGGCCTGATCTACCGCGCAATGGGCTTCC
CAACTGACTTCTTCACCGTATTGTTCGCAATCGGTCGTCTGCCAGGATGGATCGC
TCACTACCGCGAGCAGCTCGGTGCAGCAGGCAACAAGATCAACCGCCCACGCCA
GGTCTACACCGGCAACGAATCCCGCAAGTTGGTTCCTCGCGAGGAGCGCTAA
SEQ ID NO. 19-
ATGTACGAGAAGATTCAACCCCCTAGCGAAGGCAGCAAAATTCGCTTTGAAGCC
GGCAAGCCGATCGTTCCCGACAACCCGATCATTCCCTTCATTCGTGGTGACGGCG
CTGGCGTTGATATCTGGCCCGCAACTGAGCGCGTTCTCGATGCCGCTGTCGCTAA
AGCCTATGGCGGTCAGCGCAAAATCACTTGGTTCAAAGTCTACGCGGGTGATGA
AGCCTGCGACCTCTACGGCACCTACCAATATCTGCCTGAAGATACGCTGACAGCG
ATCCGCGAGTACGGCGTGGCAATCAAAGGCCCGCTGACGACGCCGATCGGTGGT
GGCATTCGATCGCTGAACGTGGCGCTACGGCAAATCTTCGATCTCTATGCCTGCG
TCCGCCCCTGTCGCTACTACACCGGCACACCCTCGCCCCACCGCACGCCCGAACA
ACTCGATGTGGTGGTCTACCGCGAAAACACCGAGGATATCTACCTCGGCATCGA
ATGGAAGCAAGGTGATCCCACCGGCGATCGCCTGATCAAGCTGCTGAACGAGGA
CTTCATTCCCAACAGCCCCAGCTTGGGTAAAAAGCAAATCCGTTTGGATTCCGGC
ATTGGTATTAAGCCGATCAGTAAAACGGGTAGCCAGCGTCTGATTCGTCGTGCGA
TCGAGCATGCCCTACGCCTCGAAGGCCGCAAGCGACATGTCACCCTTGTCCACAA
GGGCAACATCATGAAGTTCACGGAAGGTGCTTTCCGGGACTGGGGCTATGAACT
GGCCACGACTGAGTTCCGAACCGACTGTGTGACTGAACGGGAGAGCTGGATTCT
TGCCAACCAAGAAAGCAAGCCGGATCTCAGCTTGGAAGACAATGCGCGGCTCAT
CGAACCTGGCTACGACGCGATGACGCCCGAAAAGCAGGCAGCAGTGGTGGCTGA
AGTGAAAGCTGTGCTCGACAGCATCGGCGCCACCCACGGCAACGGTCAGTGGAA
GTCTAAGGTGCTGGTTGACGATCGCATTGCTGACAGCATCTTCCAGCAGATTCAA
ACCCGCCCGGGTGAATACTCGGTGCTGGCGACGATGAACCTCAATGGCGACTAC
ATCTCTGATGCAGCGGCGGCGGTGGTCGGTGGCCTGGGCATGGCCCCCGGTGCC
AACATTGGCGACGAAGCGGCGATCTTTGAAGCGACCCACGGCGCAGCGCCCAAG
CACGCTGGCCTCGATCGCATTAACCCCGGCTCGGTCATCCTCTCCGGCGTGATGA
TGCTGGAGTACCTAGGCTGGCAAGAGGCTGCTGACTTGATCACCAAGGGCATCA
GCCAAGCGATCGCTAACCGTGAGGTCACCTACGATCTGGCTCGGTTGATGGAAC
CGGCGGTTGATCAACCACTCAAGTGCTCGGAATTTGCCGAAGCCATCGTCAAGC
ATTTCGACGATTAG
SEQ ID NO. 20-
MYEKIQPPSEGSKIRFEAGKPIVPDNPIIPFIRGDGTGVDIWPATERVLDAAVAKAYGG
QRKITWFKVYAGDEACDLYGTYQYLPEDTLTAIREYGVAIKGPLTTPIGGGIRSLNVA
LRQIFDLYACVRPCRYYTGTPSPHRTPEQLDVVVYRENTEDIYLGIEWKQGDPTGDR
LIKLLNEDFIPNSPSLGKKQIRLDSGIGIKPISKTGSQRLIRRAIEHALRLEGRKRHVTLV
HKGNIMKFTEGAFRDWGYELATTEFRTDCVTERESWILANQESKPDLSLEDNARLIE
PGYDAMTPEKQAAVVAEVKAVLDSIGATHGNGQWKSKVLVDDRIADSIFQQIQTRP
GEYSVLATMNLNGDYISDAAAAVVGGLGMAPGANIGDEAAIFEATHGTAPKHAGL
DRINPGSVILSGVMMLEYLGWQEAADLITKGISQAIANREVTYDLARLMEPAVDQPL
KCSEFAEAIVKHFDD
SEQ ID NO. 21-
GTGAAAAACGTGCTGGCGATCATTCTCGGTGGAGGCGCAGGCAGTCGTCTCTATC
CACTAACCAAACAGCGCGCCAAACCAGCGGTCCCCCTGGCGGGCAAATACCGCT
TGATCGATATTCCCGTCAGCAATTGCATCAACGCTGACATCAACAAAATCTATGT
GCTGAC
GCAGTTTAACTCTGCCTCGCTCAACCGCCACCTCAGTCAGACCTACAACCTCTCC
AGCGGCTTTGGCAATGGCTTTGTTGAGGTGCTAGCAGCTCAGATTACGCCGGAGA
ACCCCAACTGGTTCCAAGGCACCGCCGATGCGGTTCGCCAGTATCTCTGGCTAAT
CAAAGAGTGGGATGTGGATGAGTACCTGATCCTGTCGGGGGATCATCTCTACCG
CATGGACTATAGCCAGTTCATTCAGCGGCACCGAGACACCAATGCCGACATCAC
ACTCTCGGTCTTGCCGATCGATGAAAAGCGCGCCTCTGATTTTGGCCTGATGAAG
CTAGATGGCAGCGGCCGGGTGGTCGAGTTCAGCGAAAAGCCCAAAGGGGATGA
ACTCAGGGCGATGCAAGTCGATACCACGATCCTCGGGCTTGACCCTGTCGCTGCT
GCTGCCCAGCCCTTCATTGCCTCGATGGGCATCTACGTCTTCAAGCGGGATGTTC
TGATCGATTTGCTCAGCCATCATCCCGAGCAAACCGACTTTGGCAAGGAAGTGAT
TCCCGCTGCAGCCACCCGCTACAACACCCAAGCCTTTCTGTTCAACGACTACTGG
GAAGACATCGGCACGATCGCCTCATTCTACGAGGCCAATCTGGCGCTGACTCAG
CAACCTAGCCCACCCTTCAGCTTCTACGACGAGCAGGCGCCGATTTACACCCGCG
CTCGCTACCTGCCGCCAACCAAGCTGCTCGATTGCCAGGTGACCCAGTCGATCAT
TGGCGAGGGCTGCATTCTCAAGCAATGCACCGTTCAGAATTCCGTCTTAGGGATT
CGCTCCCGCATTGAGGCCGACTGCGTGATCCAGGACGCCTTGTTGATGGGCGCTG
ACTTCTACGAAACCTCGGAGCTACGGCACCAGAATCGGGCCAATGGCAAAGTGC
CGATGGGAATCGGCAGTGGCAGCACCATCCGTCGCGCCATCGTCGACAAAAATG
CCCACATTGGCCAGAACGTTCAGATCGTCAACAAAGA
CCATGTGGAAGAGGCCGATCGCGAAGATCTGGGCTTTATGATCCGCAGCGGCAT
TGTCGTTGTGGTCAAAGGGGCGGTTATTCCCGACAACACGGTGATCTAA
SEQ ID NO. 22-
MKNVLAIILGGGAGSRLYPLTKQRAKPAVPLAGKYRLIDIPVSNCINADINKIYVLTQ
FNSASLNRHLSQTYNLSSGFGNGFVEVLAAQITPENPNWFQGTADAVRQYLWLIKE
WDVDEYLILSGDHLYRMDYSQFIQRHRDTNADITLSVLPIDEKRASDFGLMKLDGSG
RVVEFSEKPKGDELRAMQVDTTILGLDPVAAAAQPFIASMGIYVFKRDVLIDLLSHH
PEQTDFGKEVIPAAATRYNTQAFLFNDYWEDIGTIASFYEANLALTQQPSPPFSFYDE
QAPIYTRARYLPPTKLLDCQVTQSIIGEGCILKQCTVQNSVLGIRSRIEADCVIQDALL
MGADFYETSELRHQNRANGKVPMGIGSGSTIRRAIVDKNAHIGQNVQIVNKDHVEE
ADREDLGFMIRSGIVVVVKGAVIPDNTVI
SEQ ID NO. 23-
ATGGCACTGAATATTCCATTCAGAAATGCGTACTATCGTTTTGCATCCAGTTACT
CATTTCTCTTTTTTATTTCCTGGTCGCTGTGGTGGTCGTTATACGCTATTTGGCTGA
AAGGACATCTAGGATTAACAGGGACGGAATTAGGTACACTTTATTCGGTCAACC
AGTTTACCAGCATTCTATTTATGATGTTCTACGGCATCGTTCAGGATAAACTCGGT
CTGAAGAAACCGCTCATCTGGTGTATGAGTTTCATTCTGGTCTTGACCGGACCGT
TTATGATTTACGTTTATGAACCGTTACTGCAAAGCAATTTTTCTGTAGGTCTAATT
CTGGGGGCGCTCTTTTTTGGCCTGGGGTATCTGGCGGGATGCGGTTTGCTTGACA
GCTTCACCGAAAAAATGGCGCGAAATTTTCATTTCGAATATGGAACAGCGCGCG
CCTGGGGATCTTTTGGCTATGCTATTGGCGCGTTCTTTGCCGGTATATTTTTTAGT
ATCAGTCCCCATATCAACTTCTGGTTGGTCTCGCTATTTGGCGCTGTATTTATGAT
GATCAACATGCGTTTTAAAGATAAGGATCACCAGTGCATAGCGGCGGATGCGGG
AGGGGTAAAAAAAGAGGATTTTATCGCAGTTTTCAAGGATCGAAACTTCTGGGTT
TTCGTCATATTTATTGTGGGGACGTGGTCTTTCTATAACATTTTTGATCAACAACT
CTTTCCTGTCTTTTATGCAGGTTTATTCGAATCACACGATGTAGGAACGCGCCTGT
ATGGTTATCTCAACTCATTCCAGGTGGTACTCGAAGCGCTGTGCATGGCGATTAT
TCCTTTCTTTGTGAATCGGGTAGGGCCAAAAAATGCATTACTTATCGGTGTTGTG
ATTATGGCGTTGCGTATCCTTTCCTGCGCGTTGTTCGTTAACCCCTGGATTATTTC
ATTAGTGAAGCTGTTACATGCCATTGAGGTTCCACTTTGTGTCATATCCGTCTTCA
AATACAGCGTGGCAAACTTTGATAAGCGCCTGTCGTCGACGATCTTTCTGATTGG
TTTTCAAATTGCCAGTTCGCTTGGGATTGTGCTGCTTTCAACGCCGACTGGGATA
CTCTTTGACCACGCAGGCTACCAGACAGTTTTCTTCGCAATTTCGGGTATTGTCTG
CCTGATGTTGCTATTTGGCATTTTCTTCCTGAGTAAAAAACGCGAGCAAATAGTT
ATGGAAACGCCTGTACCTTCAGCAATATAG
SEQ ID NO> 24-
MALNIPFRNAYYRFASSYSFLFFISWSLWWSLYAIWLKGHLGLTGTELGTLYSVNQF
TSILFMMFYGIVQDKLGLKKPLIWCMSFILVLTGPFMIYVYEPLLQSNFSVGLILGALF
FGLGYLAGCGLLDSFTEKMARNFHFEYGTARAWGSFGYAIGAFFAGIFFSISPHINFW
LVSLFGAVFMMINMRFKDKDHQCIAADAGGVKKEDFIAVFKDRNFWVFVIFIVGTW
SFYNIFDQQLFPVFYAGLFE
SEQ ID NO. 25-
ATGGTGGCAGCTCAAAATCTCTACATTCTGCACATTCAGACCCATGGTCTGCTGC
GAGGGCAGAACTTGGAACTGGGGCGAGATGCCGACACCGGCGGGCAGACCAAG
TACGTCTTAGAACTGGCTCAAGCCCAAGCTAAATCCCCACAAGTCCAACAAGTC
GACATCATCACCCGCCAAATCACCGACCCCCGCGTCAGTGTTGGTTACAGTCAGG
CGATCGAACCCTTTGCGCCCAAAGGTCGGATTGTCCGTTTGCCTTTTGGCCCCAA
ACGCTACCTCCGTAAAGAGCTGCTTTGGCCCCATCTCTACACCTTTGCGGATGCA
ATTCTCCAATATCTGGCTCAGCAAAAGCGCACCCCGACTTGGATTCAGGCCCACT
ATGCTGATGCTGGCCAAGTGGGATCACTGCTGAGTCGCTGGTTGAATGTACCGCT
AATTTTCACAGGGCATTCTCTGGGGCGGATCAAGCTAAAAAAGCTGTTGGAGCA
AGACTGGCCGCTTGAGGAAATTGAAGCGCAATTCAATATTCAACAGCGAATTGA
TGCGGAGGAGATGACGCTCACTCATGCTGACTGGATTGTCGCCAGCACTCAGCA
GGAAGTGGAGGAGCAATACCGCGTTTACGATCGCTACAACCCAGAGCGCAAACT
TGTCATTCCACCGGGTGTCGATACCGATCGCTTCAGGTTTCAGCCCTTGGGCGAT
CGCGGTGTTGTTCTCCAACAGGAACTGAGCCGCTTTCTGCGCGACCCAGAAAAAC
CTCAAATTCTCTGCCTCTGTCGCCCCGCACCTCGCAAAAATGTACCGGCGCTGGT
GCGAGCCTTTGGCGAACATCCTTGGCTGCGCAAAAAAGCCAACCTTGTCTTAGTA
CTGGGCAGCCGCCAAGACATCAACCAGATGGATCGCGGCAGTCGGCAGGTGTTC
CAAGAGATTTTCCATCTGGTCGATCGCTACGACCTCTACGGCAGCGTCGCCTATC
CCAAACAGCATCAGGCTGATGATGTGCCGGAGTTCTATCGCCTAGCGGCTCATTC
CGGCGGGGTATTCGTCAATCCGGCGCTGACCGAACCTTTTGGTTTGACAATTTTG
GAGGCAGGAAGCTGCGGCGTGCCGGTGGTGGCAACCCATGATGGCGGCCCCCAG
GAAATTCTCAAACACTGTGATTTCGGCACTTTAGTTGATGTCAGCCGACCCGCTA
ATATCGCGACTGCACTCGCCACCCTGCTGAGCGATCGCGATCTTTGGCAGTGCTA
TCACCGCAATGGCATTGAAAAAGTTCCCGCCCATTACAGCTGGGATCAACATGTC
AATACCCTGTTTGAGCGCATGGAAACGGTGGCTTTGCCTCGTCGTCGTGCTGTCA
GTTTCGTACGGAGTCGCAAACGCTTGATTGATGCCAAACGCCTTGTCGTTAGTGA
CATCGACAACACACTGTTGGGCGATCGTCAAGGACTCGAGAATTTAATGACCTAT
CTCGATCAGTATCGCGATCATTTTGCCTTTGGAATTGCCACGGGGCGTCGCCTAG
ACTCTGCCCAAGAAGTCTTGAAAGAGTGGGGCGTTCCTTCGCCAAACTTCTGGGT
GACTTCCGTCGGCAGCGAGATTCACTATGGCACCGATGCTGAACCGGATATCAG
CTGGGAAAAGCATATCAATCGCAACTGGAATCCTCAGCGAATTCGGGCAGTAAT
GGCACAACTACCCTTTCTTGAACTGCAGCCGGAAGAGGATCAAACACCCTTCAA
AGTCAGCTTCTTTGTCCGCGATCGCCACGAGACTGTGCTGCGAGAAGTACGGCAA
CATCTTCGCCGCCATCGCCTGCGGCTGAAGTCAATCTATTCCCATCAGGAGTTTC
TTGACATTCTGCCGCTAGCTGCCTCGAAAGGGGATGCGATTCGCCACCTCTCACT
CCGCTGGCGGATTCCTCTTGAGAACATTTTGGTGGCAGGCGATTCTGGTAACGAT
GAGGAAATGCTCAAGGGCCATAATCTCGGCGTTGTAGTTGGCAATTACTCACCG
GAATTGGAGCCACTGCGCAGCTACGAGCGCGTCTATTTTGCTGAGGGCCACTATG
CTAATGGCATTCTGGAAGCCTTAAAACACTATCGCTTTTTTGAGGCGATCGCTTA
A
SEQ ID NO. 26-
MVAAQNLYILHIQTHGLLRGQNLELGRDADTGGQTKYVLELAQAQAKSPQVQQVDI
ITRQITDPRVSVGYSQAIEPFAPKGRIVRLPFGPKRYLRKELLWPHLYTFADAILQYLA
QQKRTPTWIQAHYADAGQVGSLLSRWLNVPLIFTGHSLGRIKLKKLLEQDWPLEEIE
AQFNIQQRIDAEEMTLTHADWIVASTQQEVEEQYRVYDRYNPERKLVIPPGVDTDRF
RFQPLGDRGVVLQQELSRFLRDPEKPQILCLCRPAPRKNVPALVRAFGEHPWLRKKA
NLVLVLGSRQDINQMDRGSRQVFQEIFHLVDRYDLYGSVAYPKQHQADDVPEFYRL
AAHSGGVFVNPALTEPFGLTILEAGSCGVPVVATHDGGPQEILKHCDFGTLVDVSRP
ANIATALATLLSDRDLWQCYHRNGIEKVPAHYSWDQHVNTLFERMETVALPRRRA
VSFVRSRKRLIDAKRLVVSDIDNTLLGDRQGLENLMTYLDQYRDHFAFGIATGRRLD
SAQEVLKEWGVPSPNFWVTSVGSEIHYGTDAEPDISWEKHINRNWNPQRIRAVMAQ
LPFLELQPEEDQTPFKVSFFVRDRHETVLREVRQHLRRHRLRLKSIYSHQEFLDILPLA
ASKGDAIRHLSLRWRIPLENILVAGDSGNDEEMLKGHNLGVVVGNYSPELEPLRSYE
RVYFAEGHYANGILEALKHYRFFEAIA
SEQ ID NO. 27-
ATGCGACAGTTATTGCTAATTTCTGACCTGGACAATACCTGGGTCGGAGATCAAC
AAGCCCTGGAACATTTGCAAGAATATCTAGGCGATCGCCGGGGAAATTTTTATTT
GGCCTATGCCACGGGGCGTTCCTACCATTCCGCGAGGGAGTTGCAAAAACAGGT
GGGACTCATGGAACCGGACTATTGGCTCACCGCGGTGGGGAGTGAAATTTACCA
TCCAGAAGGCCTGGACCAACATTGGGCTGATTACCTCTCTGAGCATTGGCAACGG
GATATCCTCCAGGCGATCGCCGATGGTTTTGAGGCCTTAAAACCCCAATCTCCCT
TGGAACAAAACCCATGGAAAATTAGCTATCATCTCGATCCCCAGGCTTGCCCCAC
CGTCATCGACCAATTAACGGAGATGTTGAAGGAAACCGGCATCCCGGTGCAGGT
GATTTTCAGCAGTGGCAAAGATGTGGATTTATTGCCCCAACGGAGTAACAAAGG
TAACGCCACCCAATATCTGCAACAACATTTAGCCATGGAGCCGTCTCAAACCCTG
GTGTGTGGGGACTCCGGCAATGATATTGGCTTATTTGAAACTTCCGCTCGGGGTG
TCATTGTCCGTAATGCCCAGCCGGAATTATTGCACTGGTATGACCAATGGGGGGA
TTCTCGTCATTATCGGGCCCAATCGAGCCATGCTGGCGCTATCCTAGAGGCGATC
GCCCATTTCGATTTTTTGAGCTGA
SEQ ID NO. 28-
MRQLLLISDLDNTWVGDQQALEHLQEYLGDRRGNFYLAYATGRSYHSARELQKQV
GLMEPDYWLTAVGSEIYHPEGLDQHWADYLSEHWQRDILQAIADGFEALKPQSPLE
QNPWKISYHLDPQACPTVIDQLTEMLKETGIPVQVIFSSGKDVDLLPQRSNKGNATQ
YLQQHLAMEPSQTLVCGDSGNDIGLFETSARGVIVRNAQPELLHWYDQWGDSRHY
RAQSSHAGAILEAIAHFDFLS
SEQ ID NO. 29-
ATGAGTGATTCCACCGCCCAACTCAGCTACGACCCCACCACGAGCTACCTCGAGC
CCAGTGGCTTGGTCTGTGAGGATGAACGGACTTCTGTGACTCCCGAGACCTTGAA
ACGGGCTTACGAGGCCCATCTCTACTACAGCCAGGGCAAAACCTCAGCGATCGC
CACCCTGCGTGATCACTACATGGCACTGGCCTACATGGTCCGCGATCGCCTCCTG
CAACGGTGGCTAGCTTCACTGTCGACCTATCAACAACAGCACGTCAAAGTGGTCT
GTTACCTGTCCGCTGAGTTTTTGATGGGTCGGCACCTCGAAAACTGCCTGATCAA
CCTGCATCTTCACGACCGCGTTCAGCAAGTTTTGGATGAACTGGGTCTCGATTTT
GAGCAACTGCTAGAGAAAGAGGAAGAACCCGGGCTAGGCAACGGTGGCCTCGG
TCGCCTCGCAGCTTGTTTCCTCGACTCCATGGCTACCCTCGACATTCCTGCCGTCG
GCTATGGCATTCGCTATGAGTTCGGTATCTTCCACCAAGAACTCCACAACGGCTG
GCAGATCGAAATCCCCGATAACTGGCTGCGCTTTGGCAACCCTTGGGAGCTAGA
GCGGCGCGAACAGGCCGTGGAAATTAAGTTGGGCGGCCACACGGAGGCCTACCA
CGATGCGCGAGGCCGCTACTGCGTCTCTTGGATCCCCGATCGCGTCATTCGCGCC
ATCCCCTACGACACCCCCGTACCGGGCTACGACACCAATAACGTCAGCATGTTGC
GGCTCTGGAAGGCTGAGGGCACCACGGAACTCAACCTTGAGGCTTTCAACTCAG
GCAACTACGACGATGCGGTTGCCGACAAAATGTCGTCGGAAACGATCTCGAAGG
TGCTCTATCCCAACGACAACACCCCCCAAGGGCGGGAACTGCGGCTGGAGCAGC
AGTATTTCTTCGTCTCGGCTTCGCTCCAAGACATCATCCGTCGCCACTTGATGAAC
CACGGTCATCTTGAGCGGCTGCATGAGGCGATCGCAGTCCAGCTTAACGACACC
CATCCCAGCGTGGCGGTGCCGGAGTTGATGCGCCTCCTGATCGATGAGCATCACC
TGACTTGGGACAATGCTTGGACGATTACACAGCGCACCTTCGCCTACACCAACCA
CACGCTGCTACCTGAAGCCTTGGAACGCTGGCCCGTGGGCATGTTCCAGCGCACT
TTACCGCGCTTGATGGAGATTATCTACGAAATCAACTGGCGCTTCTTGGCCAATG
TGCGGGCCTGGTATCCCGGTGACGACACGAGAGCTCGCCGCCTCTCCCTGATTGA
GGAAGGAGCTGAGCCCCAGGTGCGCATGGCTCACCTCGCCTGCGTGGGCAGTCA
TGCCATCAACGGTGTGGCAGCCCTGCATACGCAACTGCTCAAGCAAGAAACCCT
GCGAGATTTCTACGAGCTTTGGCCCGAGAAATTCTTCAACATGACCAACGGTGTG
ACGCCCCGCCGCTGGCTGCTGCAAAGTAATCCTCGCCTAGCCAACCTGATCAGCG
ATCGCATTGGCAATGACTGGATTCATGATCTCAGGCAACTGCGACGGCTGGAAG
ACAGCGTGAACGATCGCGAGTTTTTACAGCGCTGGGCAGAGGTCAAGCACCAAA
ATAAGGTCGATCTGAGCCGCTACATCTACCAGCAGACTCGCATAGAAGTCGATC
CGCACTCTCTCTTTGATGTGCAAGTCAAACGGATTCACGAATACAAACGCCAGCT
CCTCGCTGTCATGCATATCGTGACGCTCTACAACTGGCTGAAGCACAATCCCCAG
CTCAACCTGGTGCCGCGCACTTTTATCTTTGCGGGCAAAGCGGCCCCGGGTTACT
ACCGTGCCAAGCAAATCGTCAAACTGATCAATGCGGTCGGGAGCATCATCAACC
ATGATCCCGATGTCCAAGGGCGACTGAAGGTCGTCTTCCTACCTAACTTCAACGT
TTCCTTGGGGCAGCGCATTTATCCAGCTGCCGATTTGTCGGAGCAAATCTCAACT
GCAGGGAAAGAAGCGTCCGGCACCGGCAACATGAAGTTCACCATGAATGGCGCG
CTGACAATCGGAACCTACGATGGTGCCAACATCGAGATCCGCGAGGAAGTCGGC
CCCGAAAACTTCTTCCTGTTTGGCCTGCGAGCCGAAGATATCGCCCGACGCCAAA
GTCGGGGCTATCGACCTGTGGAGTTCTGGAGCAGCAATGCGGAACTGCGGGCAG
TCCTCGATCGCTTTAGCAGTGGTCACTTCACACCGGATCAGCCCAACCTCTTCCA
AGACTTGGTCAGCGATCTGCTGCAGCGGGATGAGTACATGTTGATGGCGGACTA
TCAGTCCTACATCGACTGCCAGCGCGAAGCTGCTGCTGCCTACCGCGATTCCGAT
CGCTGGTGGCGGATGTCGCTACTCAACACCGCGAGATCGGGCAAGTTCTCCTCCG
ATCGCACGATCGCTGACTACAGCGAACAGATCTGGGAGGTCAAACCAGTCCCCG
TCAGCCTAAGCACTAGCTTTTAG
SEQ ID NO. 30-
MSDSTAQLSYDPTTSYLEPSGLVCEDERTSVTPETLKRAYEAHLYYSQGKTSAIATLR
DHYMALAYMVRDRLLQRWLASLSTYQQQHVKVVCYLSAEFLMGRHLENCLINLHL
HDRVQQVLDELGLDFEQLLEKEEEPGLGNGGLGRLAACFLDSMATLDIPAVGYGIR
YEFGIFHQELHNGWQIEIPDNWLRFGNPWELERREQAVEIKLGGHTEAYHDARGRY
CVSWIPDRVIRAIPYDTPVPGYDTNNVSMLRLWKAEGTTELNLEAFNSGNYDDAVA
DKMSSETISKVLYPNDNTPQGRELRLEQQYFFVSASLQDIIRRHLMNHGHLERLHEAI
AVQLNDTHPSVAVPELMRLLIDEHHLTWDNAWTITQRTFAYTNHTLLPEALERWPV
GMFQRTLPRLMEIIYEINWRFLANVRAWYPGDDTRARRLSLIEEGAEPQVRMAHLA
CVGSHAINGVAALHTQLLKQETLRDFYELWPEKFFNMTNGVTPRRWLLQSNPRLAN
LISDRIGNDWIHDLRQLRRLEDSVNDREFLQRWAEVKHQNKVDLSRYIYQQTRIEVD
PHSLFDVQVKRIHEYKRQLLAVMHIVTLYNWLKHNPQLNLVPRTFIFAGKAAPGYY
RAKQIVKLINAVGSIINHDPDVQGRLKVVFLPNFNVSLGQRIYPAADLSEQISTAGKE
ASGTGNMKFTMNGALTIGTYDGANIEIREEVGPENFFLFGLRAEDIARRQSRGYRPVE
FWSSNAELRAVLDRFSSGHFTPDQPNLFQDLVSDLLQRDEYMLMADYQSYIDCQRE
AAAAYRDSDRWWRMSLLNTARSGKFSSDRTIADYSEQIWEVKPVPVSLSTSF
SEQ ID NO. 31-
ATGGCTGCCATTAATACGAAAGTCAAAAAAGCCGTTATCCCCGTTGCGGGATTA
GGAACCAGGATGTTGCCGGCGACGAAAGCCATCCCGAAAGAGATGCTGCCACTT
GTCGATAAGCCATTAATTCAATACGTCGTGAATGAATGTATTGCGGCTGGCATTA
CTGAAATTGTGCTGGTTACACACTCATCTAAAAACTCTATTGAAAACCACTTTGA
TACCAGTTTTGAACTGGAAGCAATGCTGGAAAAACGTGTAAAACGTCAACTGCT
TGATGAAGTGCAGTCTATTTGTCCACCGCACGTGACTATTATGCAAGTTCGTCAG
GGTCTGGCGAAAGGCCTGGGACACGCGGTATTGTGTGCTCACCCGGTAGTGGGT
GATGAACCGGTAGCTGTTATTTTGCCTGATGTTATTCTGGATGAATATGAATCCG
ATTTGTCACAGGATAACCTGGCAGAGATGATCCGCCGCTTTGATGAAACGGGTC
ATAGCCAGATCATGGTTGAACCGGTTGCTGATGTGACCGCATATGGCGTTGTGGA
TTGCAAAGGCGTTGAATTAGCGCCGGGTGAAAGCGTACCGATGGTTGGTGTGGT
AGAAAAACCGAAAGCGGATGTTGCGCCGTCTAATCTCGCTATTGTGGGTCGTTAC
GTACTTAGCGCGGATATTTGGCCGTTGCTGGCAAAAACCCCTCCGGGAGCTGGTG
ATGAAATTCAGCTCACCGACGCAATTGATATGCTGATCGAAAAAGAAACGGTGG
AAGCCTATCATATGAAAGGGAAGAGCCATGACTGCGGTAATAAATTAGGTTACA
TGCAGGCCTTCGTTGAATACGGTATTCGTCATAACACCCTTGGCACGGAATTTAA
AGCCTGGCTTGAAGAAGAGATGGGCATTAAGAAGTAA
SEQ ID NO. 32-
MAAINTKVKKAVIPVAGLGTRMLPATKAIPKEMLPLVDKPLIQYVVNECIAAGITEIV
LVTHSSKNSIENHFDTSFELEAMLEKRVKRQLLDEVQSICPPHVTIMQVRQGLAKGL
GHAVLCAHPVVGDEPVAVILPDVILDEYESDLSQDNLAEMIRRFDETGHSQIMVEPV
ADVTAYGVVDCKGVELAPGESVPMVGVVEKPKADVAPSNLAIVGRYVLSADIWPL
LAKTPPGAGDEIQLTDAIDMLIEKETVEAYHMKGKSHDCGNKLGYMQAFVEYGIRH
NTLGTEFKAWLEEEMGIKK
SEQ ID NO. 33-
ATGAAATCCCCCCAGGCTCAACAAATCCTAGACCAGGCCCGCCGTTTGCTCTACG
AAAAAGCCATGGTCAAAATCAATGGGCAATACGTGGGGACGGTGGCGGCCATTC
CCCAATCGGATCACCATGATTTGAACTATACGGAAGTTTTCATTCGGGACAATGT
GCCGGTGATGATCTTCTTGTTACTGCAAAATGAAACGGAAATTGTCCAAAACTTT
TTGGAAATTTGCCTCACCCTCCAAAGTAAGGGCTTTCCCACCTACGGCATTTTTCC
CACTAGTTTTGTGGAAACGGAAAACCATGAACTCAAGGCAGACTATGGCCAACG
GGCGATCGGTCGAGTTTGCTCGGTGGATGCGTCCCTCTGGTGGCCTATTTTGGCC
TATTACTACGTGCAAAGAACCGGCAATGAAGCCTGGGCTAGACAAACCCATGTG
CAATTGGGGCTACAAAAGTTTTTAAACCTCATTCTCCATCCAGTCTTTCGGGATG
CACCCACTTTGTTTGTGCCCGACGGGGCCTTTATGATTGACCGCCCCATGGATGT
GTGGGGAGCGCCGTTGGAAATCCAAACCCTGCTCTACGGAGCCCTGAAAAGTGC
GGCGGGGTTACTGTTAATCGACCTCAAGGCGAAGGGTTATTGCAGCAATAAAGA
CCATCCTTTTGACAGCTTCACGATGGAGCAGAGTCATCAATTTAACCTGAGTGTG
GATTGGCTCAAAAAACTCCGCACCTATCTGCTCAAGCATTATTGGATTAATTGCA
ATATTGTCCAAGCTCTCCGCCGCCGTCCCACGGAACAGTACGGTGAAGAAGCCA
GCAACGAACATAATGTCCACACAGAAACCATTCCCAACTGGCTCCAGGATTGGC
TCGGCGATCGGGGAGGCTATTTAATCGGCAATATCCGCACGGGTCGCCCCGATTT
TCGCTTTTTCTCCCTGGGTAATTGCTTGGGGGCAATTTTCGATGTCACTAGCTTGG
CCCAGCAACGTTCCTTTTTCCGTTTGGTATTAAATAATCAGCGGGAGTTATGTGC
CCAAATGCCCCTGAGGATTTGCCATCCCCCCCTCAAAGATGACGATTGGCGCAGT
AAAACCGGCTTTGACCGCAAAAATTTACCCTGGTGCTACCACAACGCCGGCCATT
GGCCCTGTTTATTTTGGTTTCTGGTGGTGGCGGTGCTCCGCCATAGCTGCCATTCC
AACTACGGCACGGTGGAGTATGCGGAAATGGGGAACCTAATTCGCAATAACTAT
GAGGTGCTTTTGCGCCGTTTGCCCAAGCATAAATGGGCTGAATATTTTGATGGCC
CCACGGGCTTTTGGGTCGGGCAACAATCCCGTTCCTACCAAACCTGGACCATTGT
GGGCCTATTGCTAGTACACCATTTCACAGAAGTTAACCCCGACGATGCTTTGATG
TTCGATTTGCCTAGTTTGAAAAGTTTGCATCAAGCGCTGCATTAA
SEQ ID NO. 34-
MKSPQAQQILDQARRLLYEKAMVKINGQYVGTVAAIPQSDHHDLNYTEVFIRDNVP
VMIFLLLQNETEIVQNFLEICLTLQSKGFPTYGIFPTSFVETENHELKADYGQRAIGRV
CSVDASLWWPILAYYYVQRTGNEAWARQTHVQLGLQKFLNLILHPVFRDAPTLFVP
DGAFMIDRPMDVWGAPLEIQTLLYGALKSAAGLLLIDLKAKGYCSNKDHPFDSFTM
EQSHQFNLSVDWLKKLRTYLLKHYWINCNIVQALRRRPTEQYGEEASNEHNVHTETI
PNWLQDWLGDRGGYLIGNIRTGRPDFRFFSLGNCLGAIFDVTSLAQQRSFFRLVLNN
QRELCAQMPLRICHPPLKDDDWRSKTGFDRKNLPWCYHNAGHWPCLFWFLVVAVL
RHSCHSNYGTVEYAEMGNLIRNNYEVLLRRLPKHKWAEYFDGPTGFWVGQQSRSY
QTWTIVGLLLVHHFTEVNPDDALMFDLPSLKSLHQALH
SEQ ID NO. 35-
ATGAATTCATCCCTTGTGATCCTTTACCACCGTGAGCCCTACGACGAAGTTAGGG
AAAATGGCAAAACGGTGTATCGAGAGAAAAAGAGTCCCAACGGGATTTTGCCCA
CCCTCAAAAGTTTTTTTGCCGATGCGGAACAGAGCACCTGGGTCGCATGGAAAC
AGGTTTCGCCGAAGCAAAAGGATGATTTTCAGGCGGATATGTCCATTGAAGGCC
TTGGCGATCGTTGTACGGTGCGCCGGGTGCCCCTGACGGCGGAGCAGGTAAAAA
ACTTCTATCACATCACTTCCAAGGAAGCCTTTTGGCCCATTCTCCACTCTTTCCCC
TGGCAGTTCACCTACGATTCTTCTGATTGGGATAATTTTCAGCACATTAACCGCTT
ATTTGCCGAGGCGGCCTGTGCCGATGCCGATGACAATGCATTGTTTTGGGTCCAC
GACTATAACCTCTGGTTAGCGCCCCTTTACATTCGTCAGCTCAAGCCCAACGCCA
AGATTGCCTTTTTCCACCACACCCCCTTCCCCAGCGTTGATATTTTCAATATTTTG
CCCTGGCGGGAGGCGATCGTAGAAAGCTTGCTGGCCTGTGATCTCTGTGGTTTTC
ATATTCCCCGCTACGTAGAAAATTTTGTCGCCGTGGCCCGTAGTCTCAAGCCGGT
GGAAATCACCAGACGGGTTGTGGTAGACCAAGCCTTTACCCCCTACGGTACGGC
CCTGGCGGAACCGGAACTCACCACCCAGTTGCGTTATGGCGATCGCCTCATTAAC
CTCGATGCGTTTCCCGTGGGCACCAATCCGGCAAATATCCGGGCGATCGTGGCCA
AAGAAAGTGTGCAACAAAAAGTTGCTGAAATTAAACAAGATTTAGGCGGTAAGA
GGCTAATTGTTTCCGCTGGGCGGGTGGATTACGTGAAGGGCACCAAGGAAATGT
TGATGTGCTATGAACGTCTACTGGAGCGTCGCCCCGAATTGCAGGGGGAAATTA
GCCTGGTAGTCCCCGTAGCCAAGGCCGCTGAGGGAATGCGTATTTATCGCAACG
CCCAAAACGAAATTGAACGACTGGCAGGGAAAATTAACGGTCGCTTTGCCAAAC
TGTCCTGGACACCAGTGATGCTGTTCACCTCTCCTTTAGCCTATGAGGAGCTCATT
GCCCTGTTCTGTGCCGCCGACATTGCCTGGATCACTCCCCTGCGGGATGGGCTAA
ACCTGGTGGCTAAGGAGTATGTGGTGGCTAAAAATGGCGAAGAAGGAGTTCTGA
TCCTCTCGGAATTTGCCGGTTGTGCGGTGGAACTACCCGATGCGGTGTTGACTAA
CCCCTACGCTTCCAGCCGTATGGACGAATCCATTGACCAGGCCCTGGCCATGGAC
AAAGACGAACAGAAAAAACGCATGGGGAGAATGTACGCCGCCATTAAGCGTTA
CGACGTTCAACAATGGGCCAATCACCTACTGCGGGAAGCCTACGCCGATGTGGT
ACTGGGAGAGCCCCCCCAAATGTAG
SEQ ID NO. 36-
MNSSLVILYHREPYDEVRENGKTVYREKKSPNGILPTLKSFFADAEQSTWVAWKQV
SPKQKDDFQADMSIEGLGDRCTVRRVPLTAEQVKNFYHITSKEAFWPILHSFPWQFT
YDSSDWDNFQHINRLFAEAACADADDNALFWVHDYNLWLAPLYIRQLKPNAKIAFF
HHTPFPSVDIFNILPWREAIVESLLACDLCGFHIPRYVENFVAVARSLKPVEITRRVVV
DQAFTPYGTALAEPELTTQLRYGDRLINLDAFPVGTNPANIRAIVAKESVQQKVAEIK
QDLGGKRLIVSAGRVDYVKGTKEMLMCYERLLERRPELQGEISLVVPVAKAAEGMR
IYRNAQNEIERLAGKINGRFAKLSWTPVMLFTSPLAYEELIALFCAADIAWITPLRDG
LNLVAKEYVVAKNGEEGVLILSEFAGCAVELPDAVLTNPYASSRMDESIDQALAMD
KDEQKKRMGRMYAAIKRYDVQQWANHLLREAYADVVLGEPPQM
SEQ ID NO. 37-
ATGAAGATTTTATTTGTGGCGGCGGAAGTATCCCCCCTAGCAAAGGTAGGTGGC
ATGGGGGATGTGGTGGGTTCCCTGCCTAAAGTTCTGCATCAGTTGGGCCATGATG
TCCGTGTCTTCATGCCCTACTACGGTTTCATCGGCGACAAGATTGATGTGCCCAA
GGAGCCGGTCTGGAAAGGGGAAGCCATGTTCCAGCAGTTTGCTGTTTACCAGTCC
TATCTACCGGACACCAAAATTCCTCTCTACTTGTTCGGCCATCCAGCTTTCGACTC
CCGAAGGATCTATGGCGGAGATGACGAGGCGTGGCGGTTCACTTTTTTTTCTAAC
GGGGCAGCTGAATTTGCCTGGAACCATTGGAAGCCGGAAATTATCCATTGCCAT
GATTGGCACACTGGCATGATCCCTGTTTGGATGCATCAGTCCCCAGACATCGCCA
CCGTTTTCACCATCCATAATCTTGCTTACCAAGGGCCCTGGCGGGGCTTGCTTGA
AACTATGACTTGGTGTCCTTGGTACATGCAGGGAGACAATGTGATGGCGGCGGC
GATTCAATTTGCCAATCGGGTGACTACCGTTTCTCCCACCTATGCCCAACAGATC
CAAACCCCGGCCTATGGGGAAAAGCTGGAAGGGTTATTGTCCTACCTGAGTGGT
AATTTAGTCGGTATTCTCAACGGTATTGATACGGAGATTTACAACCCGGCGGAAG
ACCGCTTTATCAGCAATGTTTTCGATGCGGACAGTTTGGACAAGCGGGTGAAAA
ATAAAATTGCCATCCAGGAGGAAACGGGGTTAGAAATTAATCGTAATGCCATGG
TGGTGGGTATAGTGGCTCGCTTGGTGGAACAAAAGGGGATTGATTTGGTGATTCA
GATCCTTGACCGCTTCATGTCCTACACCGATTCCCAGTTAATTATCCTCGGCACTG
GCGATCGCCATTACGAAACCCAACTTTGGCAGATGGCTTCCCGATTTCCTGGGCG
GATGGCGGTGCAATTACTCCACAACGATGCCCTTTCCCGTCGAGTCTATGCCGGG
GCGGATGTGTTTTTAATGCCTTCTCGCTTTGAGCCCTGTGGGCTGAGTCAATTGAT
GGCCATGCGTTATGGCTGTATCCCCATTGTGCGGCGGACAGGGGGTTTGGTGGAT
ACGGTATCCTTCTACGATCCTATCAATGAAGCCGGCACCGGCTATTGCTTTGACC
GTTATGAACCCCTGGATTGCTTTACGGCCATGGTGCGGGCCTGGGAGGGTTTCCG
TTTCAAGGCAGATTGGCAAAAATTACAGCAACGGGCCATGCGGGCAGACTTTAG
TTGGTACCGTTCCGCCGGGGAATATATCAAAGTTTATAAGGGCGTGGTGGGGAA
ACCGGAGGAATTAAGCCCCATGGAAGAGGAAAAAATCGCTGAGTTAACTGCTTC
CTATCGCTAA
SEQ ID NO. 38-
MKILFVAAEVSPLAKVGGMGDVVGSLPKVLHQLGHDVRVFMPYYGFIGDKIDVPKE
PVWKGEAMFQQFAVYQSYLPDTKIPLYLFGHPAFDSRRIYGGDDEAWRFTFFSNGA
AEFAWNHWKPEIIHCHDWHTGMIPVWMHQSPDIATVFTIHNLAYQGPWRGLLETMT
WCPWYMQGDNVMAAAIQFANRVTTVSPTYAQQIQTPAYGEKLEGLLSYLSGNLVGI
LNGIDTEIYNPAEDRFISNVFDADSLDKRVKNKIAIQEETGLEINRNAMVVGIVARLV
EQKGIDLVIQILDRFMSYTDSQLIILGTGDRHYETQLWQMASRFPGRMAVQLLHNDA
LSRRVYAGADVFLMPSRFEPCGLSQLMAMRYGCIPIVRRTGGLVDTVSFYDPINEAG
TGYCFDRYEPLDCFTAMVRAWEGFRFKADWQKLQQRAMRADFSWYRSAGEYIKV
YKGVVGKPEELSPMEEEKIAELTASYR
SEQ ID NO. 39-
TGGGCGCGGGGCATGACTATCGTCGCCGCACTTATGACTGTCTTCTTTATCATGC
AACTCGTAGGACAGGTGGTACCTACGGTTATCCACAGAATCAGGGGATAACGCA
GGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTA
CCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCA
CGTTAAGGGATTTTGGTCATGAGATTATCAAAAAGGATCTTCACCTGCTAGCGAA
GATCCTTTGATCTTTTCTACGGGGTCTGACGCTCAGTGGAACGAAAACTCACGTT
AAGGGATTTTGGTCATGAACAATAAAACTGTCTGCTTACATAAACAGTAATACAA
GGGGTGTTATGAGCCATATTCAACGGGAAACGTCTTGCTCTAGGCCGCGATTAAA
TTCCAACATGGATGCTGATTTATATGGGTATAAATGGGCTCGCGATAATGTCGGG
CAATCAGGTGCGACAATCTATCGATTGTATGGGAAGCCCGATGCGCCAGAGTTG
TTTCTGAAACATGGCAAAGGTAGCGTTGCCAATGATGTTACAGATGAGATGGTCA
GACTAAACTGGCTGACGGAATTTATGCCTCTTCCGACCATCAAGCATTTTATCCG
TACTCCTGATGATGCATGGTTACTCACCACTGCGATCCCCGGGAAAACAGCATTC
CAGGTATTAGAAGAATATCCTGATTCAGGTGAAAATATTGTTGATGCGCTGGCAG
TGTTCCTGCGCCGGTTGCATTCGATTCCTGTTTGTAATTGTCCTTTTAACAGCGAT
CGCGTATTTCGTCTCGCTCAGGCGCAATCACGAATGAATAACGGTTTGGTTGATG
CGAGTGATTTTGATGACGAGCGTAATGGCTGGCCTGTTGAACAAGTCTGGAAAG
AAATGCATAAACTTTTGCCATTCTCACCGGATTCAGTCGTCACTCATGGTGATTTC
TCACTTGATAACCTTATTTTTGACGAGGGGAAATTAATAGGTTGTATTGATGTTG
GACGAGTCGGAATCGCAGACCGATACCAGGATCTTGCCATCCTATGGAACTGCC
TCGGTGAGTTTTCTCCTTCATTACAGAAACGGCTTTTTCAAAAATATGGTATTGAT
AATCCTGATATGAATAAATTGCAGTTTCATTTGATGCTCGATGAGTTTTTCTAAGA
ATTAATTCATGAGCGGATACATATTTGAATGTATTTAGAAAAATAAACAAATAGG
GGTTCCGCGCACATTTCCCCGAAAAGTGCCACCTGAAATTGTAAACGTTAATATT
TTGTTAAAATTCGCGTTAAATTTTTGTTAAATCAGCTCATTTTTTAACCAATAGGC
CGAAATCGGCAAAATCCCTTATAAATCAAAAGAATAGACCGAGATAGGGTTGAG
TGTTGTTCCAGTTTGGAACAAGAGTCCACTATTAAAGAACGTGGACTCCAACGTC
AAAGGGCGAAAAACCGTCTATCAGGGCGATGGCCCACTACGTGAACCATCACCC
TAATCAAGTTTTTTGGGGTCGAGGTGCCGTAAAGCACTAAATCGGAACCCTAAA
GGGAGCCCCCGATTTAGAGCTTGACGGGGAAAGCCGGCGAACGTGGCGAGAAA
GGAAGGGAAGAAAGCGAAAGGAGCGGGCGCTAGGGCGCTGGCAAGTGTAGCGG
TCACGCTGCGCGTAACCACCACACCCGCCGCGCTTAATGCGCCGCTACAGGGCG
CGTCCCATTCGCCAATCCGGATATAGTTCCTCCTTTCAGCAAAAAACCCCTCAAG
ACCCGTTTAGAGGCCCCAAGGGGTTATGCTAGTTATTGCTCAGCGGTGGCAGCAG
CCAACTCAGCTTCCTTTCGGGCTTTGTTAGCAGCCGGATCTCAGTGGTGGTGGTG
GTGGTGCTCGAGTGCGGCCGCAAGCTTTCATTAATGATGATGGTGGTGGTGGCTG
CCGGTCGCACGGGTGTCGCTGTATTTCTTCAGGTCTTCCAGGTGCGCCAGACGGT
TTTCTTTGTTAATACGTTGGGTGGTGATACGATCCGGATAGCAACGCATAAACAT
GTTGGTGAAGTGCTCGCCGTAGTGGATACGTTCGTTCGCGCTCGGCTCGAACAGC
GGATACGCGCTCGCTTCGAAGTTCGGCTCGTGAAAGTACGCGCACGCGAAACGT
TCACGGGTGTTCAGTTTAACCTTGTGCGGGGTGCTCAGCAGTTGGCCACCGGTCA
TGAACTGCAGAATATCGCCCGGAAAAACGGTCCACACACCCGGGGTCGGGGTAA
CGAAGGTCCACGGTTCGTCGTGCTCAAACATGCCCGCGCTGCTCTCGCCCGGCAG
CCAGTTACGGTTACGCTTTTCGCCCTCCACCGGCGGACGGATATACAGGCCACCA
ACATCGTCTTGCGCCGCAATCACCAGCAGACCGTAGTCGGTGTGCGCACCAATAC
CACGGCTCAGGGTGCTGGTCTGCGGCGGGAAACGCAGCACACGCATGTGGTGCC
AGCCATCACGGGTCAGGTCGGTGAAGGTGTTGATCGGCAGTTCAAAACCCAGCG
CGGTCAGTTTCAGCAGACGCTCGCCCGCCAGACCCAGTTCCTCCATAAAGGTTTT
CATGCTCTTTTGATAGGTGTTGTTCGGCCACGGAACCGGACCATGGCACGGCCAA
CCCGCTTTAACACGCTGATCGCCCACGCTCAGGTCCTTGCACACGGTAAAAATTT
CCGGGAAATCCGGCTTGCCCGCGGTAACTTCCTCGCCGCTCGCCACATAACCGCT
GTAGGTCAGGTCGCTAACGCAGCTGCTTTTGAAGGTCAGCGGCTCTTTGCAAAAT
TGCTTGCTCGCCGCCATCGCTTCTTGGGTCTTACGATCCTGCTCGCTGTCGGTTTT
AATCTGGAAGATACCATCCTTTTGCCACGCCTGAATCAGCGCACGACCCAGGCTG
ATGTCCGCCGCGCAACCGGTCACTTCGGTCGGCAGTTCAAAGGTCTGCAGGTTGG
TCATGCTGGTTTCCTCTTTGTCCATGTACAGGCTCGGATGCTCATTCCACACAACG
TCCGGGCTGCATAACCCTAGTGAGGGAAATACTCCCCATCTACTTGGAGCGTGTA
TCATATGTATATCTCCTTCTTAAAGTTAAACAAAATTATTTCTAGAGGGGAATTGT
TATCCGCTCACAATTCCCCTATAGTGAGTCGTATTAATTTCGCGGGATCGAGATC
GATCTCGATCCTCTACGCCGGACGCATCGTGGCCGGCATCACCGGCGCCACAGGT
GCGGTTGCTGGCGCCTATATCGCCGACATCACCGATGGGGAAGATCGGGCTCGC
CACTTCGGGCTCATGAGCGCTTGTTTCGGCGTGGGTATGGTGGCAGGCCCCGTGG
CCGGGGGACTGTTGGGCGCCATCTCCTTGCATGCACCATTCCTTGCGGCGGCGGT
GCTCAACGGCCTCAACCTACTACTGGGCTGCTTCCTAATGCAGGAGTCGCATAAG
GGAGAGCGTCGAGATCCCGGACACCATCGAATGGCGCAAAACCTTTCGCGGTAT
GGCATGATAGCGCCCGGAAGAGAGTCAATTCAGGGTGGTGAATGTGAAACCAGT
AACGTTATACGATGTCGCAGAGTATGCCGGTGTCTCTTATCAGACCGTTTCCCGC
GTGGTGAACCAGGCCAGCCACGTTTCTGCGAAAACGCGGGAAAAAGTGGAAGCG
GCGATGGCGGAGCTGAATTACATTCCCAACCGCGTGGCACAACAACTGGCGGGC
AAACAGTCGTTGCTGATTGGCGTTGCCACCTCCAGTCTGGCCCTGCACGCGCCGT
CGCAAATTGTCGCGGCGATTAAATCTCGCGCCGATCAACTGGGTGCCAGCGTGGT
GGTGTCGATGGTAGAACGAAGCGGCGTCGAAGCCTGTAAAGCGGCGGTGCACAA
TCTTCTCGCGCAACGCGTCAGTGGGCTGATCATTAACTATCCGCTGGATGACCAG
GATGCCATTGCTGTGGAAGCTGCCTGCACTAATGTTCCGGCGTTATTTCTTGATGT
CTCTGACCAGACACCCATCAACAGTATTATTTTCTCCCATGAAGACGGTACGCGA
CTGGGCGTGGAGCATCTGGTCGCATTGGGTCACCAGCAAATCGCGCTGTTAGCG
GGCCCATTAAGTTCTGTCTCGGCGCGTCTGCGTCTGGCTGGCTGGCATAAATATC
TCACTCGCAATCAAATTCAGCCGATAGCGGAACGGGAAGGCGACTGGAGTGCCA
TGTCCGGTTTTCAACAAACCATGCAAATGCTGAATGAGGGCATCGTTCCCACTGC
GATGCTGGTTGCCAACGATCAGATGGCGCTGGGCGCAATGCGCGCCATTACCGA
GTCCGGGCTGCGCGTTGGTGCGGACATCTCGGTAGTGGGATACGACGATACCGA
AGACAGCTCATGTTATATCCCGCCGTTAACCACCATCAAACAGGATTTTCGCCTG
CTGGGGCAAACCAGCGTGGACCGCTTGCTGCAACTCTCTCAGGGCCAGGCGGTG
AAGGGCAATCAGCTGTTGCCCGTCTCACTGGTGAAAAGAAAAACCACCCTGGCG
CCCAATACGCAAACCGCCTCTCCCCGCGCGTTGGCCGATTCATTAATGCAGCTGG
CACGACAGGTTTCCCGACTGGAAAGCGGGCAGTGAGCGCAACGCAATTAATGTA
AGTTAGCTCACTCATTAGGCACCGGGATCTCGACCGATGCCCTTGAGAGCCTTCA
ACCCAGTCAGCTCCTTCCGG
SEQ ID NO. 40-
ATTTAGCGTCTTCTAATCCAGTGTAGACAGTAGTTTTGGCTCCGTTGAGCACTGTA
GCCTTGGGCGATCGCTCTAAACATTACATAAATTCACAAAGTTTTCGTTACATAA
AAATAGTGTCTACTTAGCTAAAAATTAAGGGTTTTTTACACCTTTTTGACAGTTAA
TCTCCTAGCCTAAAAAGCAAGAGTTTTTAACTAAGACTCTTGCCCTTTACAACCT
CGAAGGAGCGTCAGATCTCATATGCACCACCACCATCACCACGAAAACCTGTAC
TTTCAGGGCAAGCTTATGATTCATGCCCCCTCCCGCTGGGGCGTGTTTCCCAGTCT
GGGTCTCTGCTCCCCCGATGTGGTGTGGAACGAACACCCCAGCCTGTACATGGAT
AAGGAAGAGACCAGTATGACCAATCTGCAAACCTTTGAACTGCCCACCGAGGTG
ACCGGTTGCGCCGCCGATATTAGCCTCGGTCGCGCCCTGATTCAAGCCTGGCAAA
AGGATGGCATCTTCCAAATCAAGACCGATTCCGAACAAGATCGCAAGACCCAAG
AGGCCATGGCCGCCAGCAAACAATTTTGCAAGGAACCCCTGACCTTTAAATCCA
GCTGCGTGAGCGATCTCACCTACAGTGGCTATGTGGCCAGTGGTGAAGAGGTGA
CCGCCGGCAAGCCCGATTTTCCCGAGATTTTTACCGTGTGCAAGGATCTGAGTGT
GGGTGATCAACGCGTGAAAGCCGGTTGGCCCTGCCATGGTCCCGTGCCCTGGCCC
AACAATACCTATCAAAAATCCATGAAGACCTTTATGGAAGAACTCGGTCTGGCC
GGTGAACGCCTGCTCAAACTGACCGCCCTCGGCTTTGAGCTGCCCATTAACACCT
TTACCGATCTCACCCGCGATGGTTGGCACCACATGCGCGTGCTGCGCTTTCCTCC
CCAAACCAGCACCCTGAGCCGCGGTATTGGTGCCCACACCGATTACGGCCTGCTC
GTGATTGCCGCCCAAGATGATGTGGGCGGTCTGTATATTCGCCCTCCCGTGGAAG
GCGAGAAACGCAACCGCAATTGGCTCCCCGGCGAAAGTTCCGCCGGCATGTTTG
AACACGATGAACCCTGGACCTTTGTGACGCCCACGCCCGGCGTGTGGACCGTGTT
TCCCGGTGATATTCTGCAATTTATGACCGGCGGTCAACTGCTCTCCACGCCCCAC
AAAGTGAAGCTCAACACCCGCGAACGCTTTGCCTGCGCCTACTTTCACGAACCCA
ATTTTGAGGCCAGTGCCTATCCCCTGTTTGAACCCTCCGCCAACGAGCGCATTCA
CTACGGCGAGCACTTTACCAATATGTTTATGCGCTGCTATCCCGATCGCATTACC
ACCCAACGCATTAACAAGGAAAATCGCCTGGCCCACCTCGAGGATCTGAAAAAG
TATAGTGATACCCGCGCCACCGGTAGTGGTGCCACCAACTTTAGCCTGCTCAAAC
AAGCCGGCGATGTGGAAGAGAACCCCGGTCCCATGACCGAAAGTATTACCAGCA
ATGGCACCCTGGTGGCCAGTGATACCCGTCGCCGCGTGTGGGCCATTGTGAGTGC
CAGCAGTGGTAACCTGGTGGAGTGGTTTGATTTTTACGTGTATAGCTTTTGCAGT
CTCTACTTTGCCCACATTTTCTTTCCCAGTGGCAATACCACCACCCAACTGCTGCA
AACCGCCGGCGTGTTTGCCGCCGGTTTTCTGATGCGCCCCATTGGCGGTTGGCTC
TTTGGCCGCATTGCCGATCGTCGCGGTCGCAAGACCAGCATGCTGATTAGCGTGT
GCATGATGTGCTTTGGCTCCCTGATTATTGCCTGCCTCCCCGGCTATGATGCCATT
GGCACCTGGGCCCCCGCCCTGCTCCTGCTGGCCCGCCTCTTTCAAGGCCTGAGCG
TGGGCGGTGAATACGGCACCAGCGCCACCTATATGAGTGAAATTGCCCTGGAGG
GCCGCAAAGGTTTTTACGCCAGTTTTCAATATGTGACCCTGATTGGCGGTCAACT
GCTCGCCATTCTCGTGGTGGTGATTCTCCAACAAATTCTGACCGATTCCCAACTG
CACGAATGGGGCTGGCGCATTCCCTTTGCCATGGGTGCCGCCCTGGCCATTGTGG
CCCTGTGGCTCCGTCGCCAACTCGATGAAACCAGCCAAAAAGAGGTGCGCGCCC
TGAAAGAAGCCGGCAGTTTTAAAGGTCTCTGGCGCAACCGCAAGGCCTTTCTCAT
GGTGCTGGGCTTTACCGCCGGCGGTAGTCTGTCCTTTTACACCTTTACCACCTACA
TGCAAAAATATCTCGTGAACACCACCGGCATGCACGCCAATGTGGCCAGCGTGA
TTATGACCGCCGCCCTGTTTGTGTTTATGCTCATTCAACCCCTGATTGGCGCCCTC
AGCGATAAGATTGGTCGTCGCACCAGTATGCTGATTTTTGGCGGTATGAGTGCCC
TCTGCACCGTGCCCATTCTCACCGCCCTGCAACACGTGTCCAGCCCCTACGCCGC
CTTTGCCCTCGTGATGCTGGCCATGGTGATTGTGTCCTTTTATACCAGCATTAGTG
GCATTCTGAAGGCCGAAATGTTTCCCGCCCAAGTGCGCGCCCTGGGCGTGGGTCT
CAGTTACGCCGTGGCCAATGCCCTGTTTGGCGGTTCCGCCGAATATGTGGCCCTG
TCCCTCAAAAGCTGGGGCAGTGAGACCACCTTTTTCTGGTACGTGACCATTATGG
GTGCCCTGGCCTTTATTGTGAGCCTGATGCTCCACCGCAAAGGCAAGGGTATTCG
CCTCTAGGGTACCAGGCAAACCCATCCCCAACCCCCTGCTGGGCCTGGATAGCAC
CGGTGGTGGTCACCACCACCATCACCACTAGAGTACTGTATGCATCGAGTGCCTG
GCGGCAGTAGCGCGGTGGTCCCACCTGACCCCATGCCGAACTCAGAAGTGAAAC
GCCGTAGCGCCGATGGTAGTGTGGGGTCTCCCCATGCGAGAGTAGGGAACTGCC
AGGCATCAAATAAAACGAAAGGCTCAGTCGAAAGACTGGGCCTT
SEQ ID NO. 41-
ATTTAGCGTCTTCTAATCCAGTGTAGACAGTAGTTTTGGCTCCGTTGAGCACTGTA
GCCTTGGGCGATCGCTCTAAACATTACATAAATTCACAAAGTTTTCGTTACATAA
AAATAGTGTCTACTTAGCTAAAAATTAAGGGTTTTTTACACCTTTTTGACAGTTAA
TCTCCTAGCCTAAAAAGCAAGAGTTTTTAACTAAGACTCTTGCCCTTTACAACCT
C
SEQ ID NO. 42-
TGCCTGGCGGCAGTAGCGCGGTGGTCCCACCTGACCCCATGCCGAACTCAGAAG
TGAAACGCCGTAGCGCCGATGGTAGTGTGGGGTCTCCCCATGCGAGAGTAGGGA
ACTGCCAGGCATCAAATAAAACGAAAGGCTCAGTCGAAAGACTGGGCCTT
SEQ ID NO. 43-
GAGGTTGTAAAGGGCAAGAGTCTTAGTTAAAAACTCTTGCTTTTTAGGCTAGGAG
ATTAACTGTCAAAAAGGTGTAAAAAACCCTTAATTTTTAGCTAAGTAGACACTAT
TTTTATGTAACGAAAACTTTGTGAATTTATGTAATGTTTAGAGCGATCGCCCAAG
GCTACAGTGCTCAACGGAGCCAAAACTACTGTCTACACTGGATTAGAAGACGCT
AAATGGTACCTACGATCTCATATGATACACGCTCCAAGTAGATGGGGAGTATTTC
CCTCACTAGGGTTATGCAGCCCGGACGTTGTGTGGAATGAGCATCCGAGCCTGTA
CATGGACAAAGAGGAAACCAGCATGACCAACCTGCAGACCTTTGAACTGCCGAC
CGAAGTGACCGGTTGCGCGGCGGACATCAGCCTGGGTCGTGCGCTGATTCAGGC
GTGGCAAAAGGATGGTATCTTCCAGATTAAAACCGACAGCGAGCAGGATCGTAA
GACCCAAGAAGCGATGGCGGCGAGCAAGCAATTTTGCAAAGAGCCGCTGACCTT
CAAAAGCAGCTGCGTTAGCGACCTGACCTACAGCGGTTATGTGGCGAGCGGCGA
GGAAGTTACCGCGGGCAAGCCGGATTTCCCGGAAATTTTTACCGTGTGCAAGGA
CCTGAGCGTGGGCGATCAGCGTGTTAAAGCGGGTTGGCCGTGCCATGGTCCGGTT
CCGTGGCCGAACAACACCTATCAAAAGAGCATGAAAACCTTTATGGAGGAACTG
GGTCTGGCGGGCGAGCGTCTGCTGAAACTGACCGCGCTGGGTTTTGAACTGCCG
ATCAACACCTTCACCGACCTGACCCGTGATGGCTGGCACCACATGCGTGTGCTGC
GTTTCCCGCCGCAGACCAGCACCCTGAGCCGTGGTATTGGTGCGCACACCGACTA
CGGTCTGCTGGTGATTGCGGCGCAAGACGATGTTGGTGGCCTGTATATCCGTCCG
CCGGTGGAGGGCGAAAAGCGTAACCGTAACTGGCTGCCGGGCGAGAGCAGCGC
GGGCATGTTTGAGCACGACGAACCGTGGACCTTCGTTACCCCGACCCCGGGTGTG
TGGACCGTTTTTCCGGGCGATATTCTGCAGTTCATGACCGGTGGCCAACTGCTGA
GCACCCCGCACAAGGTTAAACTGAACACCCGTGAACGTTTCGCGTGCGCGTACTT
TCACGAGCCGAACTTCGAAGCGAGCGCGTATCCGCTGTTCGAGCCGAGCGCGAA
CGAACGTATCCACTACGGCGAGCACTTCACCAACATGTTTATGCGTTGCTATCCG
GATCGTATCACCACCCAACGTATTAACAAAGAAAACCGTCTGGCGCACCTGGAA
GACCTGAAGAAATACAGCGACACCCGTGCGACCGGCAGCCACCACCACCATCAT
CATTAATGAAAGCTTGCGGCCGCACTCGAGCACCACCACCACCACCACTGAGAT
CCGGCTGCTAACAAAGCCCGAAAGGAAGCTGAGTTGGCTGCTGCCACCGCTGAG
CAATAACTAGCATAACCCCTTGGGGCCTCTAAACGGGTCTTGAGGGGTTTTTTG
SEQ ID NO. 44-
CAAAAAACCCCTCAAGACCCGTTTAGAGGCCCCAAGGGGTTATGCTAG
SEQ ID NO. 45-
GAGCGTGTATCATATGAGATCTGACGCTCCTTCGAGGTTGTAAAGGGCAAGAGT
CTTAGTTAAAAACTCTTGCTTTTTAGGCTAGGAGATTAACTGTCAAAAAGGTGTA
AAAAACCCTTAATTTTTAGCTAAGTAGACACTATTTTTATGTAACGAAAACTTTG
TGAATTTATGTAATGTTTAGAGCGATCGCCCAAGGCTACAGTGCTCAACGGAGCC
AAAACTACTGTCTACACTGGATTAGAAGACGCTAAATGGTACCTACGATCTCATA
TGATACACGCTCCAAGTAGATGGGGAGTATTTCCCTCACTAGGGTTATGCAGCCC
GGACGTTGTGTGGAATGAGCATCCGAGCCTGTACATGGACAAAGAGGAAACCAG
CATGACCAACCTGCAGACCTTTGAACTGCCGACCGAAGTGACCGGTTGCGCGGC
GGACATCAGCCTGGGTCGTGCGCTGATTCAGGCGTGGCAAAAGGATGGTATCTT
CCAGATTAAAACCGACAGCGAGCAGGATCGTAAGACCCAAGAAGCGATGGCGG
CGAGCAAGCAATTTTGCAAAGAGCCGCTGACCTTCAAAAGCAGCTGCGTTAGCG
ACCTGACCTACAGCGGTTATGTGGCGAGCGGCGAGGAAGTTACCGCGGGCAAGC
CGGATTTCCCGGAAATTTTTACCGTGTGCAAGGACCTGAGCGTGGGCGATCAGCG
TGTTAAAGCGGGTTGGCCGTGCCATGGTCCGGTTCCGTGGCCGAACAACACCTAT
CAAAAGAGCATGAAAACCTTTATGGAGGAACTGGGTCTGGCGGGCGAGCGTCTG
CTGAAACTGACCGCGCTGGGTTTTGAACTGCCGATCAACACCTTCACCGACCTGA
CCCGTGATGGCTGGCACCACATGCGTGTGCTGCGTTTCCCGCCGCAGACCAGCAC
CCTGAGCCGTGGTATTGGTGCGCACACCGACTACGGTCTGCTGGTGATTGCGGCG
CAAGACGATGTTGGTGGCCTGTATATCCGTCCGCCGGTGGAGGGCGAAAAGCGT
AACCGTAACTGGCTGCCGGGCGAGAGCAGCGCGGGCATGTTTGAGCACGACGAA
CCGTGGACCTTCGTTACCCCGACCCCGGGTGTGTGGACCGTTTTTCCGGGCGATA
TTCTGCAGTTCATGACCGGTGGCCAACTGCTGAGCACCCCGCACAAGGTTAAACT
GAACACCCGTGAACGTTTCGCGTGCGCGTACTTTCACGAGCCGAACTTCGAAGCG
AGCGCGTATCCGCTGTTCGAGCCGAGCGCGAACGAACGTATCCACTACGGCGAG
CACTTCACCAACATGTTTATGCGTTGCTATCCGGATCGTATCACCACCCAACGTA
TTAACAAAGAAAACCGTCTGGCGCACCTGGAAGACCTGAAGAAATACAGCGACA
CCCGTGCGACCGGCAGCCACCACCACCATCATCATTAATGAAAGCTTGCGGCCG
CACTCGAGCACCACCACCACCACCACTGAGATCCGGCTGCTAACAAAGCCCGAA
AGGAAGCTGAGTTGGCTGCTGCCACCGCTGAGCAATAACTAGCATAACCCCTTG
GGGCCTCTAAACGGGTCTTGAGGGGTTTTTTGCTGAAAGGAGGAACTATATCCGG
ATTGGCGAATGGGACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCCCAAAA
AACTTGATTAGGGTGATGGTTCACGTAGTGGGCCATCGCCCTGATAGACGGTTTT
TCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCG
ATTTCGGCCTATTGGTTAAAAAATGAGCTGATTTAACAAAAATTTAACGCGAATT
TTAACAAAATATTAACGTTTACAATTTCAGGTGGCACTTTTCGGGGAAATGTGCG
CGGAACCCCTATTTGTTTATTTTTCTAAATACATTCAAATATGTATCCGCTCATGA
ATTAATTCTTAGAAAAACTCATCGAGCATCAAATGAAACTGCAATTTATTCATAT
CAGGATTATCAATACCATATTTTTGAAAAAGCCGTTTCTGTAATGAAGGAGAAAA
CTCACCGAGGCAGTTCCATAGGATGGCAAGATCCTGGTATCGGTCTGCGATTCCG
ACTCGTCCAACATCAATACAACCTATTAATTTCCCCTCGTCAAAAATAAGGTTAT
CAAGTGAGAAATCACCATGAGTGACGACTGAATCCGGTGAGAATGGCAAAAGTT
TATGCATTTCTTTCCAGACTTGTTCAACAGGCCAGCCATTACGCTCGTCATCAAA
ATCACTCGCATCAACCAAACCGTTATTCATTCGTGATTGCGCCTGAGCGAGACGA
AATACGCGATCGCTGTTAAAAGGACAATTACAAACAGGAATCGAATGCAACCGG
CGCAGGAACACTGCCAGCGCATCAACAATATTTTCACCTGAATCAGGATATTCTT
CTAATACCTGGAATGCTGTTTTCCCGGGGATCGCAGTGGTGAGTAACCATGCATC
ATCAGGAGTACGGATAAAATGCTTGATGGTCGGAAGAGGCATAAATTCCGTCAG
CCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGC
ACCTGATTGCCCGACATTATCGCGAGCCCATTTATACCCATATAAATCAGCATCC
ATGTTGGAATTTAATCGCGGCCTAGAGCAAGACGTTTCCCGTTGAATATGGCTCA
TAACACCCCTTGTATTACTGTTTATGTAAGCAGACAGTTTTATTGTTCATGACCAA
AATCCCTTAACGTGAGTTTTCGTTCCACTGAGCGTCAGACCCCGTAGAAAAGATC
AAAGGATCTTCGCTAGCAGGTGAAGATCCTTTTTGATAATCTCATGACCAAAATC
CCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTC
CGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGG
ACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGAC
TTGAGCGTCGATTTTTGTGATGCTCGTCAGGGGGGCGGAGCCTATGGAAAAACG
CCAGCAACGCGGCCTTTTTACGGTTCCTGGCCTTTTGCTGGCCTTTTGCTCACATG
TTCTTTCCTGCGTTATCCCCTGATTCTGTGGATAACCGTAGGTACCATTTAGCGTC

1. A recombinant microorganism having an improved ethylene producing ability, wherein the recombinant microorganism expresses at least one ethylene forming enzyme
(EFE) protein having an amino acid sequence at least 95% identical to SEQ ID NO:1 by expressing a non-native EFE expressing nucleotide sequence,
wherein an amount of EFE protein produced by the recombinant microorganism is greater than that produced relative to a control microorganism lacking the non-native EFE expressing nucleotide sequence.

2. The recombinant microorganism of claim 1, wherein the recombinant microorganism expresses at least one alpha-ketoglutarate permease (AKGP) protein having an amino acid sequence at least 95% identical to SEQ ID NO:2 by expressing a non-native AKGP expressing nucleotide sequence,
wherein an amount of AKGP protein produced by the recombinant microorganism is greater than that produced relative to a control microorganism lacking the non-native AKGP expressing nucleotide sequence.

3. The recombinant microorganism of claim 1, wherein the amount of EFE protein produced by the recombinant microorganism is from about 5% to about 200% or more greater than that produced relative to the control microorganism lacking the non-native EFE expressing nucleotide sequence.

4. The recombinant microorganism of claim 1, wherein the recombinant microorganism includes a microorganism selected from the group consisting of Cyanobacteria, Synechococcus, Synechococcus elongatus, Synechococcus leopoliensis, Synechocystis, Anabaena, Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.

5. The recombinant microorganism of claim 1, wherein the non-native EFE expressing nucleotide sequence is inserted into a bacterial vector plasmid, a high copy number bacterial vector plasmid, a bacterial vector plasmid having an inducible promoter, a nucleotide guide of a homologous recombination system, a CRISPR CAS system, a phage display system, or a combination thereof.

6. The recombinant microorganism of claim 2, wherein the non-native EFE, expressing nucleotide sequence and the non-native AKGP expressing nucleotide sequence are inserted into a bacterial vector plasmid, a high copy number bacterial vector plasmid, a bacterial vector plasmid having an inducible promoter, a nucleotide guide of a homologous recombination system, a CRISPR CAS system, a phage display system, or a combination thereof.

7. The recombinant microorganism of claim 1, wherein the non-native EFE, expressing nucleotide sequence has a nucleotide sequence at least 95% identical to SEQ ID NO:3, and the non-native EFE expressing nucleotide sequence is inserted into a vector plasmid of a Chlamydomonas sp. bacterium.

8. The recombinant microorganism of claim 2, wherein the non-native EFE, expressing nucleotide sequence has a nucleotide sequence at least 95% identical to SEQ ID NO:4, and the non-native EFE expressing nucleotide sequence and the AKGP expressing nucleotide sequence are inserted into a vector plasmid of an Escherichia sp. bacterium.

9. The recombinant microorganism of claim 1, further comprising a non-native AKGP expressing nucleotide sequence, wherein the non-native EFE expressing nucleotide sequence and non-native AKGP expressing nucleotide sequence express a combined amino acid sequence at least 95% identical to SEQ ID NO:5, and the non-native EFE expressing nucleotide sequence and non-native AKGP expressing nucleotide sequence are inserted into a bacterial plasmid of a Synechococcus sp. bacterium.

10. The recombinant microorganism of claim 1, further comprising a non-native AKGP expressing nucleotide sequence, wherein the non-native EFE expressing nucleotide sequence and non-native AKGP expressing nucleotide sequence express a combined amino acid sequence at least 95% identical to SEQ ID NO:6, and the non-native EFE expressing nucleotide sequence and non-native AKGP expressing nucleotide sequence are inserted into a bacterial plasmid of a Synechococcus sp. bacterium.

11. The recombinant microorganism of claim 1, wherein the recombinant microorganism expresses at least one phosphoenolpyruvate synthase (PEP) protein having an amino acid sequence at least 95% identical to SEQ ID NO:15 by expressing a non-native PEP expressing nucleotide sequence.

12. The recombinant microorganism of claim 11, wherein the recombinant microorganism expresses at least one citrate synthase protein having an amino acid sequence at least 95% identical to SEQ ID NO:17 by expressing a non-native citrate synthase expressing nucleotide sequence, wherein an amount of citrate synthase protein produced by the recombinant microorganism is greater than that produced relative to a control microorganism lacking the non-native citrate synthase expressing nucleotide sequence.

13. The recombinant microorganism of claim 1, wherein the recombinant microorganism expresses at least one isocitrate dehydrogenase (IDH) protein having an amino acid sequence at least 95% identical to SEQ ID NO:20 by expressing a non-native IDH expressing nucleotide sequence,
wherein an amount of IDH protein produced by the recombinant microorganism is greater than that produced relative to a control microorganism lacking the non-native IDH expressing nucleotide sequence, and
wherein an amount of AKG produced by the recombinant microorganism is greater than that produced relative to the control microorganism.

14. The recombinant microorganism of claim 13, wherein the recombinant microorganism contains a deletion in a glucose-1-phosphate adenylyltransferase expressing nucleotide sequence, wherein an amount of glucose-1-phosphate adenylyltransferase protein produced by the recombinant microorganism is less than that produced relative to a control microorganism lacking the deletion.

15. The recombinant microorganism of claim 11, wherein the recombinant microorganism includes a microorganism selected from the group consisting of Cyanobacteria, Synechococcus, Synechococcus elongatus, Synechococcus leopoliensis, Synechocystis, Anabaena, Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.

16. The recombinant microorganism of claim 1, wherein the recombinant microorganism expresses at least one sucrose permease protein having an amino acid sequence at least 95% identical to SEQ ID NO:24 by expressing a non-native sucrose permease expressing nucleotide sequence,
wherein an amount of sucrose permease protein produced by the recombinant microorganism is greater than that produced relative to a control microorganism lacking the non-native sucrose permease expressing nucleotide sequence.

17. The recombinant microorganism of claim 1, wherein the recombinant microorganism expresses at least one protein selected from the group consisting of sucrose phosphate synthase proteins having an amino acid sequence at least 95% identical to SEQ ID NO:26, sucrose-6-phosphatase proteins having an amino acid sequence at least 95% identical to SEQ ID NO:28, glycogen phosphorylase proteins having an amino acid sequence at least 95% identical to SEQ ID NO:30, and UTP-glucose-1-phosphate uridylyltransferase proteins having an amino acid sequence at least 95% identical to SEQ ID NO:32, by expressing a non-native nucleotide sequence encoding the at least one protein,
wherein an amount of the at least one protein produced by the recombinant microorganism is greater than that produced relative to a control microorganism lacking the non-native nucleotide sequence encoding the at least one protein,
wherein an amount of sucrose produced by the recombinant microorganism is greater than that produced relative to the control microorganism.

18. The recombinant microorganism of claim 17, wherein the recombinant microorganism contains at least one deletion in at least one nucleotide sequence, wherein the at least one nucleotide sequence encodes at least one protein selected from an invertase protein having an amino acid sequence at least 95% identical to SEQ ID NO:34, a glucosylglycerol-phosphate synthase protein having an amino acid sequence at least 95% identical to SEQ ID NO:36, and a glycogen synthase protein having an amino acid sequence at least 95% identical to SEQ ID NO:38, wherein an amount of the at least one protein produced by the recombinant microorganism is less than that produced relative to a control microorganism lacking the at least one deletion.

19. A method of producing a recombinant microorganism having an improved ethylene producing ability comprising:
producing the recombinant microorganism by inserting a non-native EFE expressing nucleotide sequence or a combined non-native EFE expressing nucleotide sequence and non-native AKGP expressing nucleotide sequence into a bacterial plasmid of a microorganism,
wherein the non-native EFE expressing nucleotide sequence has a nucleotide sequence at least 95% identical to SEQ ID NO:3 or SEQ ID NO:4, or
wherein the combined non-native EFE expressing nucleotide sequence and non-native AKGP expressing nucleotide sequence expresses an amino acid sequence at least 95% identical to SEQ ID NO:5 or SEQ ID NO:6; or
wherein the combined non-native EFE expressing nucleotide sequence and non-native AKGP expressing nucleotide sequence has a nucleotide sequence at least 95% identical to SEQ ID NO:7.

20. The method of claim 19, wherein the microorganism is selected from the group consisting of Cyanobacteria, Synechococcus Synechococcus elongatus, Synechococcus leopoliensis, Synechocystis, Anabaena, Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.

21. The method of claim 19, wherein the non-native FEE expressing nucleotide sequence has a nucleotide sequence at least 95% identical to SEQ ID NO:3 and the microorganism is a Chlamydomonas sp. bacterium; or
wherein the non-native EFE expressing nucleotide sequence has a nucleotide sequence at least 95% identical to SEQ ID NO:4 and the microorganism is an Escherichia sp. bacterium; or
the combined non-native EFE expressing nucleotide sequence and non-native AKGP expressing nucleotide sequence expresses an amino acid sequence at least 95% identical to SEQ. ID NO:5 or SEQ ID NO:6 and the microorganism is a Synechococcus sp. bacterium; or the combined non-native EFE expressing nucleotide sequence and non-native AKGP expressing nucleotide sequence has a nucleotide sequence at least 95% identical to SEQ ID NO:7 and the microorganism is Synechococcus sp. bacterium.

22. A method of producing ethylene comprising:
providing a recombinant microorganism having an improved ethylene producing ability, wherein the recombinant microorganism expresses at least one ethylene forming enzyme (EFE) protein having an amino acid sequence at least 95% identical to SEQ ID NO:1 by expressing a non-native EFE expressing nucleotide sequence,
wherein an amount of EFE protein produced by the recombinant microorganism is greater than that produced relative to a control microorganism lacking the non-native EFE expressing nucleotide sequence;
culturing the recombinant microorganism in a bioreactor culture vessel under conditions sufficient to produce ethylene in the bioreactor culture vessel; and
harvesting ethylene from the bioreactor culture vessel.

23. The method of claim 22, wherein the recombinant microorganism contains a non-native EFE expressing nucleotide sequence or a combined non-native EFE expressing nucleotide sequence and non-native AKGP expressing nucleotide sequence inserted into a bacterial plasmid of the microorganism,
wherein the non-native EFE expressing nucleotide sequence has a nucleotide sequence at least 95% identical to SEQ ID NO:3 or SEQ ID NO:4, or
wherein the combined non-native EFE expressing nucleotide sequence and non-native AKGP expressing nucleotide sequence has a nucleotide sequence at least 95% identical to SEQ ID NO:7; or
wherein the combined non-native EFE expressing nucleotide sequence and non-native AKGP expressing nucleotide sequence expresses an amino acid sequence at least 95% identical to SEQ ID NO:5 or SEQ ID NO:6.

24. The method of claim 22, wherein the microorganism is selected from the group consisting of Cyanobacteria, Synechococcus, Synechococcus elongatus, Synechococcus leopoliensis, Synechocystis, Anabaena, Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.

25. The method of claim 22, further comprising increasing an amount of ethylene production by adding at least one activator to a culture containing the recombinant microorganism located within the bioreactor culture vessel; or adding CO₂ to a culture atmosphere contained within the bioreactor culture vessel at rate of between about 100 ml/minute and about 500 ml/minute.

26. The method of claim 22, further comprising decreasing an amount of ethylene production by removing at least one molecular switch from the cell culture containing the recombinant microorganism located within the bioreactor culture vessel.

27. The method of claim 22, further comprising controlling the amount of ethylene produced from the microbial culture by increasing or decreasing the concentration of at least one nutrient or the amount of at least one stimulus when culturing the recombinant microorganism.

28. The method of claim 22, wherein the concentration of at least one nutrient and the amount of at least one stimulus are at a ratio of from about 0.5-1.5 gr./liter to about 0.1 mM in the microbial culture.

29. The method of claim 22, further comprising removing the amount of ethylene produced from the microbial culture by condensing the ethylene from a gaseous to a liquid state, or wherein the amount of ethylene recovered is from about 0.5 ml to about 10 ml/liter/h.

30. A recombinant microorganism having an improved alpha-ketoglutarate (AKG) producing ability,
wherein the recombinant microorganism expresses at least one phosphoenolpyruvate synthase (PEP) protein having an amino acid sequence at least 95% identical to SEQ ID NO:15 by expressing a non-native PEP expressing nucleotide sequence, and
wherein an amount of PEP protein produced by the recombinant microorganism is greater than that produced relative to a control microorganism lacking the non-native PEP expressing nucleotide sequence, or
wherein the recombinant microorganism expresses at least one isocitrate dehydrogenase (IDH) protein having an amino acid sequence at least 95% identical to SEQ ID NO:20 by expressing a non-native IDH expressing nucleotide sequence,
wherein an amount of IDH protein produced by the recombinant microorganism is greater than that produced relative to a control microorganism lacking the non-native IDH expressing nucleotide sequence, and
wherein an amount of AKG produced by the recombinant microorganism is greater than that produced relative to the control microorganism.

31. The recombinant microorganism of claim 30, wherein the recombinant microorganism expresses at least one citrate synthase protein having an amino acid sequence at least 95% identical to SEQ ID NO:17 by expressing a non-native citrate synthase expressing nucleotide sequence, wherein an amount of citrate synthase protein produced by the recombinant microorganism is greater than that produced relative to a control microorganism lacking the non-native citrate synthase expressing nucleotide sequence.

32. The recombinant microorganism of claim 30, wherein the recombinant microorganism contains a deletion in a glucose-1-phosphate adenylyltransferase expressing nucleotide sequence, wherein an amount of glucose-1-phosphate adenylyltransferase protein produced by the recombinant microorganism is less than that produced relative to a control microorganism lacking the deletion.

33. The recombinant microorganism of claim 30, wherein the recombinant microorganism includes a microorganism selected from the group consisting of Cyanobacteria, Synechococcus, Synechococcus elongatus, Synechococcus leopoliensis, Synechocystis, Anabaena, Pseudomonas, Pseudomonas syringae, Pseudomonas savastanoi, Chlamydomonas, Chlamydomonas reinhardtii, Escherichia, Escherichia coli, Geobacteria, algae, microalgae, electrosynthesis bacteria, a photosynthetic microorganism, yeast, filamentous fungi, and a plant cell.


SEQUENCE LISTING (46 sequences)

SEQ ID NO: 1 - protein, 387 aa, Pseudomonas savastanoi
MIHAPSRWGVFPSLGLCSPDVVWNEHPSLYMDKEETSMTNLQTFELPTEVTGCAADISLGRALIQAWQKDGIFQIKTDSEQDRKTQEAMAASKQFCKEPLTFKSSCVSDLTYSGYVASGEEVTAGKPDFPEIFTVCKDLSVGDQRVKAGWPCHGPVPWPNNTYQKSMKTFMEELGLAGERLLKLTALGFELPINTFTDLTRDGWHHMRVLRFPPQTSTLSRGIGAHTDYGLLVIAAQDDVGGLYIRPPVEGEKRNRNWLPGESSAGMFEHDEPWTFVTPTPGVWTVFPGDILQFMTGGQLLSTPHKVKLNTRERFACAYFHEPNFEASAYPLFEPSANERIHYGEHFTNMFMRCYPDRITTQRINKENRLAHLEDLKKYSDTRATGS

SEQ ID NO: 2 - protein, 431 aa, Pseudomonas syringae
MTESITSNGTLVASDTRRRVWAIVSASSGNLVEWFDFYVYSFCSLYFAHIFFPSGNTTTQLLQTAGVFAAGFLMRPIGGWLFGRIADRRGRKTSMLISVCMMCFGSLIIACLPGYDAIGTWAPALLLLARLFQGLSVGGEYGTSATYMSEIALEGRKGFYASFQYVTLIGGQLLAILVVVILQQILTDSQLHEWGWRIPFAMGAALAIVALWLRRQLDETSQKEVRALKEAGSFKGLWRNRKAFLMVLGFTAGGSLSFYTFTTYMQKYLVNTTGMHANVASVIMTAALFVFMLIQPLIGALSDKIGRRTSMLIFGGMSALCTVPILTALQHVSSPYAAFALVMLAMVIVSFYTSISGILKAEMFPAQVRALGVGLSYAVANALFGGSAEYVALSLKSWGSETTFFWYVTIMGALAFIVSLMLHRKGKGIRL

SEQ ID NO: 3 - DNA, 1161 bp, Artificial Sequence
  Description of Artificial Sequence Synthetic polynucleotide
atgattcacgccccgtcgcgctggggcgtgtttccctcgctgggcctgtgcagccccgacgtggtgtggaacgagcacccgagcctgtacatggacaaggaggagacgtcgatgaccaacctgcagacgttcgagctgccgaccgaggtgaccggctgcgccgccgacatctccctgggccgggcgctgatccaggcgtggcagaaggacggcatcttccagatcaagaccgacagcgagcaggaccggaagacccaggaggcgatggcggcctccaagcagttctgcaaggagcccctgaccttcaagtcgtcctgcgtcagcgacctgacctactcgggctacgtggcctcgggcgaggaggtgaccgccggcaagccggactttccggagatcttcaccgtgtgcaaggacctgagcgtgggcgaccagcgggtcaaggcgggctggccctgccacggccccgtgccgtggccgaacaacacctaccagaagtccatgaagacgttcatggaggagctgggcctggccggcgagcgcctgctgaagctgaccgcgctgggcttcgagctgcccatcaacacgttcaccgacctgacccgggacggctggcaccacatgcgcgtcctgcggtttccgccccagaccagcacgctgagccgcggcattggcgcgcacacggactacggcctgctggtgattgccgcgcaggacgacgtgggcggcctgtacattcgcccgccggtggagggcgagaagcgcaaccggaactggctgcccggcgagtcctcggcgggcatgttcgagcacgacgagccctggacgttcgtgacccccacgccgggcgtgtggacggtgtttcccggcgacatcctgcagttcatgaccggcggccagctgctgtcgacgccgcacaaggtgaagctgaacacccgggagcgcttcgcctgcgcgtacttccacgagccgaacttcgaggcctcggcctaccccctgttcgagccctccgcgaacgagcgcatccactacggcgagcacttcaccaatatgtttatgcgctgctaccccgaccgcatcaccacccagcgcatcaacaaggagaatcgcctggcgcacctggaggacctgaagaagtacagcgacacccgcgccaccggctcg

SEQ ID NO: 4 - DNA, 1161 bp, Artificial Sequence
  Description of Artificial Sequence Synthetic polynucleotide
atgatacacgctccaagtagatggggagtatttccctcactagggttatgcagcccggacgttgtgtggaatgagcatccgagcctgtacatggacaaagaggaaaccagcatgaccaacctgcagacctttgaactgccgaccgaagtgaccggttgcgcggcggacatcagcctgggtcgtgcgctgattcaggcgtggcaaaaggatggtatcttccagattaaaaccgacagcgagcaggatcgtaagacccaagaagcgatggcggcgagcaagcaattttgcaaagagccgctgaccttcaaaagcagctgcgttagcgacctgacctacagcggttatgtggcgagcggcgaggaagttaccgcgggcaagccggatttcccggaaatttttaccgtgtgcaaggacctgagcgtgggcgatcagcgtgttaaagcgggttggccgtgccatggtccggttccgtggccgaacaacacctatcaaaagagcatgaaaacctttatggaggaactgggtctggcgggcgagcgtctgctgaaactgaccgcgctgggttttgaactgccgatcaacaccttcaccgacctgacccgtgatggctggcaccacatgcgtgtgctgcgtttcccgccgcagaccagcaccctgagccgtggtattggtgcgcacaccgactacggtctgctggtgattgcggcgcaagacgatgttggtggcctgtatatccgtccgccggtggagggcgaaaagcgtaaccgtaactggctgccgggcgagagcagcgcgggcatgtttgagcacgacgaaccgtggaccttcgttaccccgaccccgggtgtgtggaccgtttttccgggcgatattctgcagttcatgaccggtggccaactgctgagcaccccgcacaaggttaaactgaacacccgtgaacgtttcgcgtgcgcgtactttcacgagccgaacttcgaagcgagcgcgtatccgctgttcgagccgagcgcgaacgaacgtatccactacggcgagcacttcaccaacatgtttatgcgttgctatccggatcgtatcaccacccaacgtattaacaaagaaaaccgtctggcgcacctggaagacctgaagaaatacagcgacacccgtgcgaccggcagc

SEQ ID NO: 5 - protein, 838 aa, Artificial Sequence
  Description of Artificial Sequence Synthetic polypeptide
MIHAPSRWGVFPSLGLCSPDVVWNEHPSLYMDKEETSMTNLQTFELPTEVTGCAADISLGRALIQAWQKDGIFQIKTDSEQDRKTQEAMAASKQFCKEPLTFKSSCVSDLTYSGYVASGEEVTAGKPDFPEIFTVCKDLSVGDQRVKAGWPCHGPVPWPNNTYQKSMKTFMEELGLAGERLLKLTALGFELPINTFTDLTRDGWHHMRVLRFPPQTSTLSRGIGAHTDYGLLVIAAQDDVGGLYIRPPVEGEKRNRNWLPGESSAGMFEHDEPWTFVTPTPGVWTVFPGDILQFMTGGQLLSTPHKVKLNTRERFACAYFHEPNFEASAYPLFEPSANERIHYGEHFTNMFMRCYPDRITTQRINKENRLAHLEDLKKYSDTRATGSGATNFSLLKQAGDVEENPGPMTESITSNGTLVASDTRRRVWAIVSASSGNLVEWFDFYVYSFCSLYFAHIFFPSGNTTTQLLQTAGVFAAGFLMRPIGGWLFGRIADRRGRKTSMLISVCMMCFGSLIIACLPGYDAIGTWAPALLLLARLFQGLSVGGEYGTSATYMSEIALEGRKGFYASFQYVTLIGGQLLAILVVVILQQILTDSQLHEWGWRIPFAMGAALAIVALWLRRQLDETSQKEVRALKEAGSFKGLWRNRKAFLMVLGFTAGGSLSFYTFTTYMQKYLVNTTGMHANVASVIMTAALFVFMLIQPLIGALSDKIGRRTSMLIFGGMSALCTVPILTALQHVSSPYAAFALVMLAMVIVSFYTSISGILKAEMFPAQVRALGVGLSYAVANALFGGSAEYVALSLKSWGSETTFFWYVTIMGALAFIVSLMLHRKGKGIRL

SEQ ID NO: 6 - protein, 854 aa, Artificial Sequence
  Description of Artificial Sequence Synthetic polypeptide
MHHHHHHENLYFQGKLMIHAPSRWGVFPSLGLCSPDVVWNEHPSLYMDKEETSMTNLQTFELPTEVTGCAADISLGRALIQAWQKDGIFQIKTDSEQDRKTQEAMAASKQFCKEPLTFKSSCVSDLTYSGYVASGEEVTAGKPDFPEIFTVCKDLSVGDQRVKAGWPCHGPVPWPNNTYQKSMKTFMEELGLAGERLLKLTALGFELPINTFTDLTRDGWHHMRVLRFPPQTSTLSRGIGAHTDYGLLVIAAQDDVGGLYIRPPVEGEKRNRNWLPGESSAGMFEHDEPWTFVTPTPGVWTVFPGDILQFMTGGQLLSTPHKVKLNTRERFACAYFHEPNFEASAYPLFEPSANERIHYGEHFTNMFMRCYPDRITTQRINKENRLAHLEDLKKYSDTRATGSGATNFSLLKQAGDVEENPGPMTESITSNGTLVASDTRRRVWAIVSASSGNLVEWFDFYVYSFCSLYFAHIFFPSGNTTTQLLQTAGVFAAGFLMRPIGGWLFGRIADRRGRKTSMLISVCMMCFGSLIIACLPGYDAIGTWAPALLLLARLFQGLSVGGEYGTSATYMSEIALEGRKGFYASFQYVTLIGGQLLAILVVVILQQILTDSQLHEWGWRIPFAMGAALAIVALWLRRQLDETSQKEVRALKEAGSFKGLWRNRKAFLMVLGFTAGGSLSFYTFTTYMQKYLVNTTGMHANVASVIMTAALFVFMLIQPLIGALSDKIGRRTSMLIFGGMSALCTVPILTALQHVSSPYAAFALVMLAMVIVSFYTSISGILKAEMFPAQVRALGVGLSYAVANALFGGSAEYVALSLKSWGSETTFFWYVTIMGALAFIVSLMLHRKGKGIRL

SEQ ID NO: 7 - DNA, 2517 bp, Artificial Sequence
  Description of Artificial Sequence Synthetic polynucleotide
atgattcatgccccctcccgctggggcgtgtttcccagtctgggtctctgctcccccgatgtggtgtggaacgaacaccccagcctgtacatggataaggaagagaccagtatgaccaatctgcaaacctttgaactgcccaccgaggtgaccggttgcgccgccgatattagcctcggtcgcgccctgattcaagcctggcaaaaggatggcatcttccaaatcaagaccgattccgaacaagatcgcaagacccaagaggccatggccgccagcaaacaattttgcaaggaacccctgacctttaaatccagctgcgtgagcgatctcacctacagtggctatgtggccagtggtgaagaggtgaccgccggcaagcccgattttcccgagatttttaccgtgtgcaaggatctgagtgtgggtgatcaacgcgtgaaagccggttggccctgccatggtcccgtgccctggcccaacaatacctatcaaaaatccatgaagacctttatggaagaactcggtctggccggtgaacgcctgctcaaactgaccgccctcggctttgagctgcccattaacacctttaccgatctcacccgcgatggttggcaccacatgcgcgtgctgcgctttcctccccaaaccagcaccctgagccgcggtattggtgcccacaccgattacggcctgctcgtgattgccgcccaagatgatgtgggcggtctgtatattcgccctcccgtggaaggcgagaaacgcaaccgcaattggctccccggcgaaagttccgccggcatgtttgaacacgatgaaccctggacctttgtgacgcccacgcccggcgtgtggaccgtgtttcccggtgatattctgcaatttatgaccggcggtcaactgctctccacgccccacaaagtgaagctcaacacccgcgaacgctttgcctgcgcctactttcacgaacccaattttgaggccagtgcctatcccctgtttgaaccctccgccaacgagcgcattcactacggcgagcactttaccaatatgtttatgcgctgctatcccgatcgcattaccacccaacgcattaacaaggaaaatcgcctggcccacctcgaggatctgaaaaagtatagtgatacccgcgccaccggtagtggtgccaccaactttagcctgctcaaacaagccggcgatgtggaagagaaccccggtcccatgaccgaaagtattaccagcaatggcaccctggtggccagtgatacccgtcgccgcgtgtgggccattgtgagtgccagcagtggtaacctggtggagtggtttgatttttacgtgtatagcttttgcagtctctactttgcccacattttctttcccagtggcaataccaccacccaactgctgcaaaccgccggcgtgtttgccgccggttttctgatgcgccccattggcggttggctctttggccgcattgccgatcgtcgcggtcgcaagaccagcatgctgattagcgtgtgcatgatgtgctttggctccctgattattgcctgcctccccggctatgatgccattggcacctgggcccccgccctgctcctgctggcccgcctctttcaaggcctgagcgtgggcggtgaatacggcaccagcgccacctatatgagtgaaattgccctggagggccgcaaaggtttttacgccagttttcaatatgtgaccctgattggcggtcaactgctcgccattctcgtggtggtgattctccaacaaattctgaccgattcccaactgcacgaatggggctggcgcattccctttgccatgggtgccgccctggccattgtggccctgtggctccgtcgccaactcgatgaaaccagccaaaaagaggtgcgcgccctgaaagaagccggcagttttaaaggtctctggcgcaaccgcaaggcctttctcatggtgctgggctttaccgccggcggtagtctgtccttttacacctttaccacctacatgcaaaaatatctcgtgaacaccaccggcatgcacgccaatgtggccagcgtgattatgaccgccgccctgtttgtgtttatgctcattcaacccctgattggcgccctcagcgataagattggtcgtcgcaccagtatgctgatttttggcggtatgagtgccctctgcaccgtgcccattctcaccgccctgcaacacgtgtccagcccctacgccgcctttgccctcgtgatgctggccatggtgattgtgtccttttataccagcattagtggcattctgaaggccgaaatgtttcccgcccaagtgcgcgccctgggcgtgggtctcagttacgccgtggccaatgccctgtttggcggttccgccgaatatgtggccctgtccctcaaaagctggggcagtgagaccacctttttctggtacgtgaccattatgggtgccctggcctttattgtgagcctgatgctccaccgcaaaggcaagggtattcgcctctag

SEQ ID NO: 8 - DNA, 6 bp, Artificial Sequence
  Description of Artificial Sequence Synthetic oligonucleotide
catatg

SEQ ID NO: 9 - DNA, 30 bp, Artificial Sequence
  Description of Artificial Sequence Synthetic oligonucleotide
caccaccaccatcatcattaatgaaagctt

SEQ ID NO: 10 - protein, 16 aa, Artificial Sequence
  Description of Artificial Sequence Synthetic peptide
MHHHHHHENLYFQGKL

SEQ ID NO: 11 - protein, 20 aa, Artificial Sequence
  Description of Artificial Sequence Synthetic peptide
GATNFSLLKQAGDVEENPGP

SEQ ID NO: 12 - DNA, 6 bp, Artificial Sequence
  Description of Artificial Sequence Synthetic oligonucleotide
aagctt

SEQ ID NO: 13 - DNA, 6 bp, Artificial Sequence
  Description of Artificial Sequence Synthetic oligonucleotide
ggtacc

SEQ ID NO: 14 - DNA, 2757 bp, Synechocystis sp.
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaaacttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaacgttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttaccctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcggtgcgcaactccggc

SEQ ID NO: 15 - protein, 919 aa, Synechocystis sp.
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVNVRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFLPSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTAVRNSG

SEQ ID NO: 16 - DNA, 1314 bp, Synechocystis sp.
atgtttgaaagggatatcgtggctactgataacaacaaggctgtcctgcactaccccggtggcgagttcgaaatggacatcatcgaggcttctgagggtaacaacggtgttgtcctgggcaagatgctgtctgagactggactgatcacttttgacccaggttatgtgagcactggctccaccgagtcgaagatcacctacatcgatggcgatgcgggaatcctgcgttaccgcggctatgacatcgctgatctggctgagaatgccaccttcaacgaggtttcttacctacttatcaacggtgagctaccaaccccagatgagcttcacaagtttaacgacgagattcgccaccacacccttctggacgaggacttcaagtcccagttcaacgtgttcccacgcgacgctcacccaatggcaaccttggcttcctcggttaacattttgtctacctactaccaggaccagctgaacccactcgatgaggcacagcttgataaggcaaccgttcgcctcatggcaaaggttccaatgctggctgcgtacgcacaccgcgcacgcaagggtgctccttacatgtacccagacaactccctcaatgcgcgtgagaacttcctgcgcatgatgttcggttacccaaccgagccatacgagatcgacccaatcatggtcaaggctctggacaagctgctcatcctgcacgctgaccacgagcagaactgctccacctccaccgttcgtatgatcggttccgcacaggccaacatgtttgtctccatcgctggtggcatcaacgctctgtccggcccactgcacggtggcgcaaaccaggctgttctggagatgctcgaagacatcaagagcaaccacggtggcgacgcaaccgagttcatgaacaaggtcaagaacaaggaagacggcgtccgcctcatgggcttcggacaccgcgtttacaagaactacgatccacgtgcagcaatcgtcaaggagaccgcacacgagatcctcgagcacctcggtggcgacgatcttctggatctggcaatcaagctggaagaaattgcactggctgatgattacttcatctcccgcaagctctacccgaacgtagacttctacaccggcctgatctaccgcgcaatgggcttcccaactgacttcttcaccgtattgttcgcaatcggtcgtctgccaggatggatcgctcactaccgcgagcagctcggtgcagcaggcaacaagatcaaccgcccacgccaggtctacaccggcaacgaatcccgcaagttggttcctcgcgaggagcgctaa

SEQ ID NO: 17 - protein, 437 aa, Synechocystis sp. [sequence omitted]

SEQ ID NO: 18 - DNA, 4148 bp, Artificial Sequence
  Description of Artificial Sequence Synthetic polynucleotide
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaaacttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaacgttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttaccctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcggtgcgcaactccggcgccaccaactcaactccggcgccaccaactttagcctgctcaaacaagccggcgatgtggaagagaaccccggtcccatgtttgaaagggatatcgtggctactgataacaacaaggctgtcctgcactaccccggtggcgagttcgaaatggacatcatcgaggcttctgagggtaacaacggtgttgtcctgggcaagatgctgtctgagactggactgatcacttttgacccaggttatgtgagcactggctccaccgagtcgaagatcacctacatcgatggcgatgcgggaatcctgcgttaccgcggctatgacatcgctgatctggctgagaatgccaccttcaacgaggtttcttacctacttatcaacggtgagctaccaaccccagatgagcttcacaagtttaacgacgagattcgccaccacacccttctggacgaggacttcaagtcccagttcaacgtgttcccacgcgacgctcacccaatggcaaccttggcttcctcggttaacattttgtctacctactaccaggaccagctgaacccactcgatgaggcacagcttgataaggcaaccgttcgcctcatggcaaaggttccaatgctggctgcgtacgcacaccgcgcacgcaagggtgctccttacatgtacccagacaactccctcaatgcgcgtgagaacttcctgcgcatgatgttcggttacccaaccgagccatacgagatcgacccaatcatggtcaaggctctggacaagctgctcatcctgcacgctgaccacgagcagaactgctccacctccaccgttcgtatgatcggttccgcacaggccaacatgtttgtctccatcgctggtggcatcaacgctctgtccggcccactgcacggtggcgcaaaccaggctgttctggagatgctcgaagacatcaagagcaaccacggtggcgacgcaaccgagttcatgaacaaggtcaagaacaaggaagacggcgtccgcctcatgggcttcggacaccgcgtttacaagaactacgatccacgtgcagcaatcgtcaaggagaccgcacacgagatcctcgagcacctcggtggcgacgatcttctggatctggcaatcaagctggaagaaattgcactggctgatgattacttcatctcccgcaagctctacccgaacgtagacttctacaccggcctgatctaccgcgcaatgggcttcccaactgacttcttcaccgtattgttcgcaatcggtcgtctgccaggatggatcgctcactaccgcgagcagctcggtgcagcaggcaacaagatcaaccgcccacgccaggtctacaccggcaacgaatcccgcaagttggttcctcgcgaggagcgctaa

SEQ ID NO: 19 - DNA, 1428 bp, Artificial Sequence
  Description of Artificial Sequence Synthetic polynucleotide
atgtacgagaagattcaaccccctagcgaaggcagcaaaattcgctttgaagccggcaagccgatcgttcccgacaacccgatcattcccttcattcgtggtgacggcgctggcgttgatatctggcccgcaactgagcgcgttctcgatgccgctgtcgctaaagcctatggcggtcagcgcaaaatcacttggttcaaagtctacgcgggtgatgaagcctgcgacctctacggcacctaccaatatctgcctgaagatacgctgacagcgatccgcgagtacggcgtggcaatcaaaggcccgctgacgacgccgatcggtggtggcattcgatcgctgaacgtggcgctacggcaaatcttcgatctctatgcctgcgtccgcccctgtcgctactacaccggcacaccctcgccccaccgcacgcccgaacaactcgatgtggtggtctaccgcgaaaacaccgaggatatctacctcggcatcgaatggaagcaaggtgatcccaccggcgatcgcctgatcaagctgctgaacgaggacttcattcccaacagccccagcttgggtaaaaagcaaatccgtttggattccggcattggtattaagccgatcagtaaaacgggtagccagcgtctgattcgtcgtgcgatcgagcatgccctacgcctcgaaggccgcaagcgacatgtcacccttgtccacaagggcaacatcatgaagttcacggaaggtgctttccgggactggggctatgaactggccacgactgagttccgaaccgactgtgtgactgaacgggagagctggattcttgccaaccaagaaagcaagccggatctcagcttggaagacaatgcgcggctcatcgaacctggctacgacgcgatgacgcccgaaaagcaggcagcagtggtggctgaagtgaaagctgtgctcgacagcatcggcgccacccacggcaacggtcagtggaagtctaaggtgctggttgacgatcgcattgctgacagcatcttccagcagattcaaacccgcccgggtgaatactcggtgctggcgacgatgaacctcaatggcgactacatctctgatgcagcggcggcggtggtcggtggcctgggcatggcccccggtgccaacattggcgacgaagcggcgatctttgaagcgacccacggcgcagcgcccaagcacgctggcctcgatcgcattaaccccggctcggtcatcctctccggcgtgatgatgctggagtacctaggctggcaagaggctgctgacttgatcaccaagggcatcagccaagcgatcgctaaccgtgaggtcacctacgatctggctcggttgatggaaccggcggttgatcaaccactcaagtgctcggaatttgccgaagccatcgtcaagcatttcgacgattag

SEQ ID NO: 20 - protein, 475 aa, Artificial Sequence
  Description of Artificial Sequence Synthetic polypeptide
MYEKIQPPSEGSKIRFEAGKPIVPDNPIIPFIRGDGTGVDIWPATERVLDAAVAKAYGGQRKITWFKVYAGDEACDLYGTYQYLPEDTLTAIREYGVAIKGPLTTPIGGGIRSLNVALRQIFDLYACVRPCRYYTGTPSPHRTPEQLDVVVYRENTEDIYLGIEWKQGDPTGDRLIKLLNEDFIPNSPSLGKKQIRLDSGIGIKPISKTGSQRLIRRAIEHALRLEGRKRHVTLVHKGNIMKFTEGAFRDWGYELATTEFRTDCVTERESWILANQESKPDLSLEDNARLIEPGYDAMTPEKQAAVVAEVKAVLDSIGATHGNGQWKSKVLVDDRIADSIFQQIQTRPGEYSVLATMNLNGDYISDAAAAVVGGLGMAPGANIGDEAAIFEATHGTAPKHAGLDRINPGSVILSGVMMLEYLGWQEAADLITKGISQAIANREVTYDLARLMEPAVDQPLKCSEFAEAIVKHFDD

SEQ ID NO: 21 - DNA, 1293 bp, Synechococcus elongatus
gtgaaaaacgtgctggcgatcattctcggtggaggcgcaggcagtcgtctctatccactaaccaaacagcgcgccaaaccagcggtccccctggcgggcaaataccgcttgatcgatattcccgtcagcaattgcatcaacgctgacatcaacaaaatctatgtgctgacgcagtttaactctgcctcgctcaaccgccacctcagtcagacctacaacctctccagcggctttggcaatggctttgttgaggtgctagcagctcagattacgccggagaaccccaactggttccaaggcaccgccgatgcggttcgccagtatctctggctaatcaaagagtgggatgtggatgagtacctgatcctgtcgggggatcatctctaccgcatggactatagccagttcattcagcggcaccgagacaccaatgccgacatcacactctcggtcttgccgatcgatgaaaagcgcgcctctgattttggcctgatgaagctagatggcagcggccgggtggtcgagttcagcgaaaagcccaaaggggatgaactcagggcgatgcaagtcgataccacgatcctcgggcttgaccctgtcgctgctgctgcccagcccttcattgcctcgatgggcatctacgtcttcaagcgggatgttctgatcgatttgctcagccatcatcccgagcaaaccgactttggcaaggaagtgattcccgctgcagccacccgctacaacacccaagcctttctgttcaacgactactgggaagacatcggcacgatcgcctcattctacgaggccaatctggcgctgactcagcaacctagcccacccttcagcttctacgacgagcaggcgccgatttacacccgcgctcgctacctgccgccaaccaagctgctcgattgccaggtgacccagtcgatcattggcgagggctgcattctcaagcaatgcaccgttcagaattccgtcttagggattcgctcccgcattgaggccgactgcgtgatccaggacgccttgttgatgggcgctgacttctacgaaacctcggagctacggcaccagaatcgggccaatggcaaagtgccgatgggaatcggcagtggcagcaccatccgtcgcgccatcgtcgacaaaaatgcccacattggccagaacgttcagatcgtcaacaaagaccatgtggaagaggccgatcgcgaagatctgggctttatgatccgcagcggcattgtcgttgtggtcaaaggggcggttattcccgacaacacggtgatctaa

SEQ ID NO: 22 - protein, 430 aa, Synechococcus elongatus
MKNVLAIILGGGAGSRLYPLTKQRAKPAVPLAGKYRLIDIPVSNCINADINKIYVLTQFNSASLNRHLSQTYNLSSGFGNGFVEVLAAQITPENPNWFQGTADAVRQYLWLIKEWDVDEYLILSGDHLYRMDYSQFIQRHRDTNADITLSVLPIDEKRASDFGLMKLDGSGRVVEFSEKPKGDELRAMQVDTTILGLDPVAAAAQPFIASMGIYVFKRDVLIDLLSHHPEQTDFGKEVIPAAATRYNTQAFLFNDYWEDIGTIASFYEANLALTQQPSPPFSFYDEQAPIYTRARYLPPTKLLDCQVTQSIIGEGCILKQCTVQNSVLGIRSRIEADCVIQDALLMGADFYETSELRHQNRANGKVPMGIGSGSTIRRAIVDKNAHIGQNVQIVNKDHVEEADREDLGFMIRSGIVVVVKGAVIPDNTVI

SEQ ID NO: 23 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctaggattaacagggacggaattaggtacactttattcggtcaaccagtttaccagcattctatttatgatgttctacggcatcgttcaggataaactcggtctgaagaaaccgctcatctggtgtatgagtttcattctggtcttgaccggaccgtttatgatttacgtttatgaaccgttactgcaaagcaatttttctgtaggtctaattctgggggcgctcttttttggcctggggtatctggcgggatgcggtttgcttgacagcttcaccgaaaaaatggcgcgaaattttcatttcgaatatggaacagcgcgcgcctggggatcttttggctatgctattggcgcgttctttgccggtatattttttagtatcagtccccatatcaacttctggttggtctcgctatttggcgctgtatttatgatgatcaacatgcgttttaaagataaggatcaccagtgcatagcggcggatgcgggaggggtaaaaaaagaggattttatcgcagttttcaaggatcgaaacttctgggttttcgtcatatttattgtggggacgtggtctttctataacatttttgatcaacaactctttcctgtcttttatgcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggtgttgtgattatggcgttgcgtatcctttcctgcgcgttgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatag

SEQ ID NO: 24 - protein, 250 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCIAADAGGVKKEDFIAVFKDRNFWVFVIFIVGTWSFYNIFDQQLFPVFYAGLFE

SEQ ID NO: 25 - DNA, 2133 bp, Synechococcus sp.
atggtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaaacttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaa

SEQ ID NO: 26 - protein, 710 aa, Synechococcus sp.
MVAAQNLYILHIQTHGLLRGQNLELGRDADTGGQTKYVLELAQAQAKSPQVQQVDIITRQITDPRVSVGYSQAIEPFAPKGRIVRLPFGPKRYLRKELLWPHLYTFADAILQYLAQQKRTPTWIQAHYADAGQVGSLLSRWLNVPLIFTGHSLGRIKLKKLLEQDWPLEEIEAQFNIQQRIDAEEMTLTHADWIVASTQQEVEEQYRVYDRYNPERKLVIPPGVDTDRFRFQPLGDRGVVLQQELSRFLRDPEKPQILCLCRPAPRKNVPALVRAFGEHPWLRKKANLVLVLGSRQDINQMDRGSRQVFQEIFHLVDRYDLYGSVAYPKQHQADDVPEFYRLAAHSGGVFVNPALTEPFGLTILEAGSCGVPVVATHDGGPQEILKHCDFGTLVDVSRPANIATALATLLSDRDLWQCYHRNGIEKVPAHYSWDQHVNTLFERMETVALPRRRAVSFVRSRKRLIDAKRLVVSDIDNTLLGDRQGLENLMTYLDQYRDHFAFGIATGRRLDSAQEVLKEWGVPSPNFWVTSVGSEIHYGTDAEPDISWEKHINRNWNPQRIRAVMAQLPFLELQPEEDQTPFKVSFFVRDRHETVLREVRQHLRRHRLRLKSIYSHQEFLDILPLAASKGDAIRHLSLRWRIPLENILVAGDSGNDEEMLKGHNLGVVVGNYSPELEPLRSYERVYFAEGHYANGILEALKHYRFFEAIA

SEQ ID NO: 27 - DNA, 735 bp, Synechocystis sp.
atgcgacagttattgctaatttctgacctggacaatacctgggtcggagatcaacaagccctggaacatttgcaagaatatctaggcgatcgccggggaaatttttatttggcctatgccacggggcgttcctaccattccgcgagggagttgcaaaaacaggtgggactcatggaaccggactattggctcaccgcggtggggagtgaaatttaccatccagaaggcctggaccaacattgggctgattacctctctgagcattggcaacgggatatcctccaggcgatcgccgatggttttgaggccttaaaaccccaatctcccttggaacaaaacccatggaaaattagctatcatctcgatccccaggcttgccccaccgtcatcgaccaattaacggagatgttgaaggaaaccggcatcccggtgcaggtgattttcagcagtggcaaagatgtggatttattgccccaacggagtaacaaaggtaacgccacccaatatctgcaacaacatttagccatggagccgtctcaaaccctggtgtgtggggactccggcaatgatattggcttatttgaaacttccgctcggggtgtcattgtccgtaatgcccagccggaattattgcactggtatgaccaatggggggattctcgtcattatcgggcccaatcgagccatgctggcgctatcctagaggcgatcgcccatttcgattttttgagctga

SEQ ID NO: 28 - protein, 244 aa, Synechocystis sp.
MRQLLLISDLDNTWVGDQQALEHLQEYLGDRRGNFYLAYATGRSYHSARELQKQVGLMEPDYWLTAVGSEIYHPEGLDQHWADYLSEHWQRDILQAIADGFEALKPQSPLEQNPWKISYHLDPQACPTVIDQLTEMLKETGIPVQVIFSSGKDVDLLPQRSNKGNATQYLQQHLAMEPSQTLVCGDSGNDIGLFETSARGVIVRNAQPELLHWYDQWGDSRHYRAQSSHAGAILEAIAHFDFLS

SEQ ID NO: 29 - DNA, 2535 bp, Synechococcus elongatus
atgagtgattccaccgcccaactcagctacgaccccaccacgagctacctcgagcccagtggcttggtctgtgaggatgaacggacttctgtgactcccgagaccttgaaacgggcttacgaggcccatctctactacagccagggcaaaacctcagcgatcgccaccctgcgtgatcactacatggcactggcctacatggtccgcgatcgcctcctgcaacggtggctagcttcactgtcgacctatcaacaacagcacgtcaaagtggtctgttacctgtccgctgagtttttgatgggtcggcacctcgaaaactgcctgatcaacctgcatcttcacgaccgcgttcagcaagttttggatgaactgggtctcgattttgagcaactgctagagaaagaggaagaacccgggctaggcaacggtggcctcggtcgcctcgcagcttgtttcctcgactccatggctaccctcgacattcctgccgtcggctatggcattcgctatgagttcggtatcttccaccaagaactccacaacggctggcagatcgaaatccccgataactggctgcgctttggcaacccttgggagctagagcggcgcgaacaggccgtggaaattaagttgggcggccacacggaggcctaccacgatgcgcgaggccgctactgcgtctcttggatccccgatcgcgtcattcgcgccatcccctacgacacccccgtaccgggctacgacaccaataacgtcagcatgttgcggctctggaaggctgagggcaccacggaactcaaccttgaggctttcaactcaggcaactacgacgatgcggttgccgacaaaatgtcgtcggaaacgatctcgaaggtgctctatcccaacgacaacaccccccaagggcgggaactgcggctggagcagcagtatttcttcgtctcggcttcgctccaagacatcatccgtcgccacttgatgaaccacggtcatcttgagcggctgcatgaggcgatcgcagtccagcttaacgacacccatcccagcgtggcggtgccggagttgatgcgcctcctgatcgatgagcatcacctgacttgggacaatgcttggacgattacacagcgcaccttcgcctacaccaaccacacgctgctacctgaagccttggaacgctggcccgtgggcatgttccagcgcactttaccgcgcttgatggagattatctacgaaatcaactggcgcttcttggccaatgtgcgggcctggtatcccggtgacgacacgagagctcgccgcctctccctgattgaggaaggagctgagccccaggtgcgcatggctcacctcgcctgcgtgggcagtcatgccatcaacggtgtggcagccctgcatacgcaactgctcaagcaagaaaccctgcgagatttctacgagctttggcccgagaaattcttcaacatgaccaacggtgtgacgccccgccgctggctgctgcaaagtaatcctcgcctagccaacctgatcagcgatcgcattggcaatgactggattcatgatctcaggcaactgcgacggctggaagacagcgtgaacgatcgcgagtttttacagcgctgggcagaggtcaagcaccaaaataaggtcgatctgagccgctacatctaccagcagactcgcatagaagtcgatccgcactctctctttgatgtgcaagtcaaacggattcacgaatacaaacgccagctcctcgctgtcatgcatatcgtgacgctctacaactggctgaagcacaatccccagctcaacctggtgccgcgcacttttatctttgcgggcaaagcggccccgggttactaccgtgccaagcaaatcgtcaaactgatcaatgcggtcgggagcatcatcaaccatgatcccgatgtccaagggcgactgaaggtcgtcttcctacctaacttcaacgtttccttggggcagcgcatttatccagctgccgatttgtcggagcaaatctcaactgcagggaaagaagcgtccggcaccggcaacatgaagttcaccatgaatggcgcgctgacaatcggaacctacgatggtgccaacatcgagatccgcgaggaagtcggccccgaaaacttcttcctgtttggcctgcgagccgaagatatcgcccgacgccaaagtcggggctatcgacctgtggagttctggagcagcaatgcggaactgcgggcagtcctcgatcgctttagcagtggtcacttcacaccggatcagcccaacctcttccaagacttggtcagcgatctgctgcagcgggatgagtacatgttgatggcggactatcagtcctacatcgactgccagcgcgaagctgctgctgcctaccgcgattccgatcgctggtggcggatgtcgctactcaacaccgcgagatcgggcaagttctcctccgatcgcacgatcgctgactacagcgaacagatctgggaggtcaaaccagtccccgtcagcctaagcactagcttttag

SEQ ID NO: 30 - protein, 844 aa, Synechococcus elongatus
MSDSTAQLSYDPTTSYLEPSGLVCEDERTSVTPETLKRAYEAHLYYSQGKTSAIATLRDHYMALAYMVRDRLLQRWLASLSTYQQQHVKVVCYLSAEFLMGRHLENCLINLHLHDRVQQVLDELGLDFEQLLEKEEEPGLGNGGLGRLAACFLDSMATLDIPAVGYGIRYEFGIFHQELHNGWQIEIPDNWLRFGNPWELERREQAVEIKLGGHTEAYHDARGRYCVSWIPDRVIRAIPYDTPVPGYDTNNVSMLRLWKAEGTTELNLEAFNSGNYDDAVADKMSSETISKVLYPNDNTPQGRELRLEQQYFFVSASLQDIIRRHLMNHGHLERLHEAIAVQLNDTHPSVAVPELMRLLIDEHHLTWDNAWTITQRTFAYTNHTLLPEALERWPVGMFQRTLPRLMEIIYEINWRFLANVRAWYPGDDTRARRLSLIEEGAEPQVRMAHLACVGSHAINGVAALHTQLLKQETLRDFYELWPEKFFNMTNGVTPRRWLLQSNPRLANLISDRIGNDWIHDLRQLRRLEDSVNDREFLQRWAEVKHQNKVDLSRYIYQQTRIEVDPHSLFDVQVKRIHEYKRQLLAVMHIVTLYNWLKHNPQLNLVPRTFIFAGKAAPGYYRAKQIVKLINAVGSIINHDPDVQGRLKVVFLPNFNVSLGQRIYPAADLSEQISTAGKEASGTGNMKFTMNGALTIGTYDGANIEIREEVGPENFFLFGLRAEDIARRQSRGYRPVEFWSSNAELRAVLDRFSSGHFTPDQPNLFQDLVSDLLQRDEYMLMADYQSYIDCQREAAAAYRDSDRWWRMSLLNTARSGKFSSDRTIADYSEQIWEVKPVPVSLSTSF

SEQ ID NO: 31 - DNA, 909 bp, Escherichia coli
atggctgccattaatacgaaagtcaaaaaagccgttatccccgttgcgggattaggaaccaggatgttgccggcgacgaaagccatcccgaaagagatgctgccacttgtcgataagccattaattcaatacgtcgtgaatgaatgtattgcggctggcattactgaaattgtgctggttacacactcatctaaaaactctattgaaaaccactttgataccagttttgaactggaagcaatgctggaaaaacgtgtaaaacgtcaactgcttgatgaagtgcagtctatttgtccaccgcacgtgactattatgcaagttcgtcagggtctggcgaaaggcctgggacacgcggtattgtgtgctcacccggtagtgggtgatgaaccggtagctgttattttgcctgatgttattctggatgaatatgaatccgatttgtcacaggataacctggcagagatgatccgccgctttgatgaaacgggtcatagccagatcatggttgaaccggttgctgatgtgaccgcatatggcgttgtggattgcaaaggcgttgaattagcgccgggtgaaagcgtaccgatggttggtgtggtagaaaaaccgaaagcggatgttgcgccgtctaatctcgctattgtgggtcgttacgtacttagcgcggatatttggccgttgctggcaaaaacccctccgggagctggtgatgaaattcagctcaccgacgcaattgatatgctgatcgaaaaagaaacggtggaagcctatcatatgaaagggaagagccatgactgcggtaataaattaggttacatgcaggccttcgttgaatacggtattcgtcataacacccttggcacggaatttaaagcctggcttgaagaagagatgggcattaagaagtaa

SEQ ID NO: 32 - protein, 302 aa, Escherichia coli
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 33 - DNA, 1470 bp, Synechocystis sp.
atgaaatccccccaggctcaacaaatcctagaccaggcccgccgtttgctctacgaaaaagccatggtcaaaatcaatgggcaatacgtggggacggtggcggccattccccaatcggatcaccatgatttgaactatacggaagttttcattcgggacaatgtgccggtgatgatcttcttgttactgcaaaatgaaacggaaattgtccaaaactttttggaaatttgcctcaccctccaaagtaagggctttcccacctacggcatttttcccactagttttgtggaaacggaaaaccatgaactcaaggcagactatggccaacgggcgatcggtcgagtttgctcggtggatgcgtccctctggtggcctattttggcctattactacgtgcaaagaaccggcaatgaagcctgggctagacaaacccatgtgcaattggggctacaaaagtttttaaacctcattctccatccagtctttcgggatgcacccactttgtttgtgcccgacggggcctttatgattgaccgccccatggatgtgtggggagcgccgttggaaatccaaaccctgctctacggagccctgaaaagtgcggcggggttactgttaatcgacctcaaggcgaagggttattgcagcaataaagaccatccttttgacagcttcacgatggagcagagtcatcaatttaacctgagtgtggattggctcaaaaaactccgcacctatctgctcaagcattattggattaattgcaatattgtccaagctctccgccgccgtcccacggaacagtacggtgaagaagccagcaacgaacataatgtccacacagaaaccattcccaactggctccaggattggctcggcgatcggggaggctatttaatcggcaatatccgcacgggtcgccccgattttcgctttttctccctgggtaattgcttgggggcaattttcgatgtcactagcttggcccagcaacgttcctttttccgtttggtattaaataatcagcgggagttatgtgcccaaatgcccctgaggatttgccatccccccctcaaagatgacgattggcgcagtaaaaccggctttgaccgcaaaaatttaccctggtgctaccacaacgccggccattggccctgtttattttggtttctggtggtggcggtgctccgccatagctgccattccaactacggcacggtggagtatgcggaaatggggaacctaattcgcaataactatgaggtgcttttgcgccgtttgcccaagcataaatgggctgaatattttgatggccccacgggcttttgggtcgggcaacaatcccgttcctaccaaacctggaccattgtgggcctattgctagtacaccatttcacagaagttaaccccgacgatgctttgatgttcgatttgcctagtttgaaaagtttgcatcaagcgctgcattaa

SEQ ID NO: 34 - protein, 489 aa, Synechocystis sp.
MKSPQAQQILDQARRLLYEKAMVKINGQYVGTVAAIPQSDHHDLNYTEVFIRDNVPVMIFLLLQNETEIVQNFLEICLTLQSKGFPTYGIFPTSFVETENHELKADYGQRAIGRVCSVDASLWWPILAYYYVQRTGNEAWARQTHVQLGLQKFLNLILHPVFRDAPTLFVPDGAFMIDRPMDVWGAPLEIQTLLYGALKSAAGLLLIDLKAKGYCSNKDHPFDSFTMEQSHQFNLSVDWLKKLRTYLLKHYWINCNIVQALRRRPTEQYGEEASNEHNVHTETIPNWLQDWLGDRGGYLIGNIRTGRPDFRFFSLGNCLGAIFDVTSLAQQRSFFRLVLNNQRELCAQMPLRICHPPLKDDDWRSKTGFDRKNLPWCYHNAGHWPCLFWFLVVAVLRHSCHSNYGTVEYAEMGNLIRNNYEVLLRRLPKHKWAEYFDGPTGFWVGQQSRSYQTWTIVGLLLVHHFTEVNPDDALMFDLPSLKSLHQALH

SEQ ID NO: 35 - DNA, 1500 bp, Synechocystis sp.
atgaattcatcccttgtgatcctttaccaccgtgagccctacgacgaagttagggaaaatggcaaaacggtgtatcgagagaaaaagagtcccaacgggattttgcccaccctcaaaagtttttttgccgatgcggaacagagcacctgggtcgcatggaaacaggtttcgccgaagcaaaaggatgattttcaggcggatatgtccattgaaggccttggcgatcgttgtacggtgcgccgggtgcccctgacggcggagcaggtaaaaaacttctatcacatcacttccaaggaagccttttggcccattctccactctttcccctggcagttcacctacgattcttctgattgggataattttcagcacattaaccgcttatttgccgaggcggcctgtgccgatgccgatgacaatgcattgttttgggtccacgactataacctctggttagcgcccctttacattcgtcagctcaagcccaacgccaagattgcctttttccaccacacccccttccccagcgttgatattttcaatattttgccctggcgggaggcgatcgtagaaagcttgctggcctgtgatctctgtggttttcatattccccgctacgtagaaaattttgtcgccgtggcccgtagtctcaagccggtggaaatcaccagacgggttgtggtagaccaagcctttaccccctacggtacggccctggcggaaccggaactcaccacccagttgcgttatggcgatcgcctcattaacctcgatgcgtttcccgtgggcaccaatccggcaaatatccgggcgatcgtggccaaagaaagtgtgcaacaaaaagttgctgaaattaaacaagatttaggcggtaagaggctaattgtttccgctgggcgggtggattacgtgaagggcaccaaggaaatgttgatgtgctatgaacgtctactggagcgtcgccccgaattgcagggggaaattagcctggtagtccccgtagccaaggccgctgagggaatgcgtatttatcgcaacgcccaaaacgaaattgaacgactggcagggaaaattaacggtcgctttgccaaactgtcctggacaccagtgatgctgttcacctctcctttagcctatgaggagctcattgccctgttctgtgccgccgacattgcctggatcactcccctgcgggatgggctaaacctggtggctaaggagtatgtggtggctaaaaatggcgaagaaggagttctgatcctctcggaatttgccggttgtgcggtggaactacccgatgcggtgttgactaacccctacgcttccagccgtatggacgaatccattgaccaggccctggccatggacaaagacgaacagaaaaaacgcatggggagaatgtacgccgccattaagcgttacgacgttcaacaatgggccaatcacctactgcgggaagcctacgccgatgtggtactgggagagcccccccaaatgtag

SEQ ID NO: 36 - protein, 499 aa, Synechocystis sp.
MNSSLVILYHREPYDEVRENGKTVYREKKSPNGILPTLKSFFADAEQSTWVAWKQVSPKQKDDFQADMSIEGLGDRCTVRRVPLTAEQVKNFYHITSKEAFWPILHSFPWQFTYDSSDWDNFQHINRLFAEAACADADDNALFWVHDYNLWLAPLYIRQLKPNAKIAFFHHTPFPSVDIFNILPWREAIVESLLACDLCGFHIPRYVENFVAVARSLKPVEITRRVVVDQAFTPYGTALAEPELTTQLRYGDRLINLDAFPVGTNPANIRAIVAKESVQQKVAEIKQDLGGKRLIVSAGRVDYVKGTKEMLMCYERLLERRPELQGEISLVVPVAKAAEGMRIYRNAQNEIERLAGKINGRFAKLSWTPVMLFTSPLAYEELIALFCAADIAWITPLRDGLNLVAKEYVVAKNGEEGVLILSEFAGCAVELPDAVLTNPYASSRMDESIDQALAMDKDEQKKRMGRMYAAIKRYDVQQWANHLLREAYADVVLGEPPQM

SEQ ID NO: 37 - DNA, 1434 bp, Synechocystis sp.
atgaagattttatttgtggcggcggaagtatcccccctagcaaaggtaggtggcatgggggatgtggtgggttccctgcctaaagttctgcatcagttgggccatgatgtccgtgtcttcatgccctactacggtttcatcggcgacaagattgatgtgcccaaggagccggtctggaaaggggaagccatgttccagcagtttgctgtttaccagtcctatctaccggacaccaaaattcctctctacttgttcggccatccagctttcgactcccgaaggatctatggcggagatgacgaggcgtggcggttcacttttttttctaacggggcagctgaatttgcctggaaccattggaagccggaaattatccattgccatgattggcacactggcatgatccctgtttggatgcatcagtccccagacatcgccaccgttttcaccatccataatcttgcttaccaagggccctggcggggcttgcttgaaactatgacttggtgtccttggtacatgcagggagacaatgtgatggcggcggcgattcaatttgccaatcgggtgactaccgtttctcccacctatgcccaacagatccaaaccccggcctatggggaaaagctggaagggttattgtcctacctgagtggtaatttagtcggtattctcaacggtattgatacggagatttacaacccggcggaagaccgctttatcagcaatgttttcgatgcggacagtttggacaagcgggtgaaaaataaaattgccatccaggaggaaacggggttagaaattaatcgtaatgccatggtggtgggtatagtggctcgcttggtggaacaaaaggggattgatttggtgattcagatccttgaccgcttcatgtcctacaccgattcccagttaattatcctcggcactggcgatcgccattacgaaacccaactttggcagatggcttcccgatttcctgggcggatggcggtgcaattactccacaacgatgccctttcccgtcgagtctatgccggggcggatgtgtttttaatgccttctcgctttgagccctgtgggctgagtcaattgatggccatgcgttatggctgtatccccattgtgcggcggacagggggtttggtggatacggtatccttctacgatcctatcaatgaagccggcaccggctattgctttgaccgttatgaacccctggattgctttacggccatggtgcgggcctgggagggtttccgtttcaaggcagattggcaaaaattacagcaacgggccatgcgggcagactttagttggtaccgttccgccggggaatatatcaaagtttataagggcgtggtggggaaaccggaggaattaagccccatggaagaggaaaaaatcgctgagttaactgcttcctatcgctaa

SEQ ID NO: 38 - protein, 477 aa, Synechocystis sp.
MKILFVAAEVSPLAKVGGMGDVVGSLPKVLHQLGHDVRVFMPYYGFIGDKIDVPKEPVWKGEAMFQQFAVYQSYLPDTKIPLYLFGHPAFDSRRIYGGDDEAWRFTFFSNGAAEFAWNHWKPEIIHCHDWHTGMIPVWMHQSPDIATVFTIHNLAYQGPWRGLLETMTWCPWYMQGDNVMAAAIQFANRVTTVSPTYAQQIQTPAYGEKLEGLLSYLSGNLVGILNGIDTEIYNPAEDRFISNVFDADSLDKRVKNKIAIQEETGLEINRNAMVVGIVARLVEQKGIDLVIQILDRFMSYTDSQLIILGTGDRHYETQLWQMASRFPGRMAVQLLHNDALSRRVYAGADVFLMPSRFEPCGLSQLMAMRYGCIPIVRRTGGLVDTVSFYDPINEAGTGYCFDRYEPLDCFTAMVRAWEGFRFKADWQKLQQRAMRADFSWYRSAGEYIKVYKGVVGKPEELSPMEEEKIAELTASYR

SEQ ID NO: 39 - DNA, 5386 bp, Artificial Sequence
  Description of Artificial Sequence Synthetic polynucleotide
tgggcgcggggcatgactatcgtcgccgcacttatgactgtcttctttatcatgcaactcgtaggacaggtggtacctacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctgctagcgaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagctttcattaatgatgatggtggtggtggctgccggtcgcacgggtgtcgctgtatttcttcaggtcttccaggtgcgccagacggttttctttgttaatacgttgggtggtgatacgatccggatagcaacgcataaacatgttggtgaagtgctcgccgtagtggatacgttcgttcgcgctcggctcgaacagcggatacgcgctcgcttcgaagttcggctcgtgaaagtacgcgcacgcgaaacgttcacgggtgttcagtttaaccttgtgcggggtgctcagcagttggccaccggtcatgaactgcagaatatcgcccggaaaaacggtccacacacccggggtcggggtaacgaaggtccacggttcgtcgtgctcaaacatgcccgcgctgctctcgcccggcagccagttacggttacgcttttcgccctccaccggcggacggatatacaggccaccaacatcgtcttgcgccgcaatcaccagcagaccgtagtcggtgtgcgcaccaataccacggctcagggtgctggtctgcggcgggaaacgcagcacacgcatgtggtgccagccatcacgggtcaggtcggtgaaggtgttgatcggcagttcaaaacccagcgcggtcagtttcagcagacgctcgcccgccagacccagttcctccataaaggttttcatgctcttttgataggtgttgttcggccacggaaccggaccatggcacggccaacccgctttaacacgctgatcgcccacgctcaggtccttgcacacggtaaaaatttccgggaaatccggcttgcccgcggtaacttcctcgccgctcgccacataaccgctgtaggtcaggtcgctaacgcagctgcttttgaaggtcagcggctctttgcaaaattgcttgctcgccgccatcgcttcttgggtcttacgatcctgctcgctgtcggttttaatctggaagataccatccttttgccacgcctgaatcagcgcacgacccaggctgatgtccgccgcgcaaccggtcacttcggtcggcagttcaaaggtctgcaggttggtcatgctggtttcctctttgtccatgtacaggctcggatgctcattccacacaacgtccgggctgcataaccctagtgagggaaatactccccatctacttggagcgtgtatcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccgg

SEQ ID NO: 40 - DNA, 3063 bp, Artificial Sequence
  Description of Artificial Sequence Synthetic polynucleotide
atttagcgtcttctaatccagtgtagacagtagttttggctccgttgagcactgtagccttgggcgatcgctctaaacattacataaattcacaaagttttcgttacataaaaatagtgtctacttagctaaaaattaagggttttttacacctttttgacagttaatctcctagcctaaaaagcaagagtttttaactaagactcttgccctttacaacctcgaaggagcgtcagatctcatatgcaccaccaccatcaccacgaaaacctgtactttcagggcaagcttatgattcatgccccctcccgctggggcgtgtttcccagtctgggtctctgctcccccgatgtggtgtggaacgaacaccccagcctgtacatggataaggaagagaccagtatgaccaatctgcaaacctttgaactgcccaccgaggtgaccggttgcgccgccgatattagcctcggtcgcgccctgattcaagcctggcaaaaggatggcatcttccaaatcaagaccgattccgaacaagatcgcaagacccaagaggccatggccgccagcaaacaattttgcaaggaacccctgacctttaaatccagctgcgtgagcgatctcacctacagtggctatgtggccagtggtgaagaggtgaccgccggcaagcccgattttcccgagatttttaccgtgtgcaaggatctgagtgtgggtgatcaacgcgtgaaagccggttggccctgccatggtcccgtgccctggcccaacaatacctatcaaaaatccatgaagacctttatggaagaactcggtctggccggtgaacgcctgctcaaactgaccgccctcggctttgagctgcccattaacacctttaccgatctcacccgcgatggttggcaccacatgcgcgtgctgcgctttcctccccaaaccagcaccctgagccgcggtattggtgcccacaccgattacggcctgctcgtgattgccgcccaagatgatgtgggcggtctgtatattcgccctcccgtggaaggcgagaaacgcaaccgcaattggctccccggcgaaagttccgccggcatgtttgaacacgatgaaccctggacctttgtgacgcccacgcccggcgtgtggaccgtgtttcccggtgatattctgcaatttatgaccggcggtcaactgctctccacgccccacaaagtgaagctcaacacccgcgaacgctttgcctgcgcctactttcacgaacccaattttgaggccagtgcctatcccctgtttgaaccctccgccaacgagcgcattcactacggcgagcactttaccaatatgtttatgcgctgctatcccgatcgcattaccacccaacgcattaacaaggaaaatcgcctggcccacctcgaggatctgaaaaagtatagtgatacccgcgccaccggtagtggtgccaccaactttagcctgctcaaacaagccggcgatgtggaagagaaccccggtcccatgaccgaaagtattaccagcaatggcaccctggtggccagtgatacccgtcgccgcgtgtgggccattgtgagtgccagcagtggtaacctggtggagtggtttgatttttacgtgtatagcttttgcagtctctactttgcccacattttctttcccagtggcaataccaccacccaactgctgcaaaccgccggcgtgtttgccgccggttttctgatgcgccccattggcggttggctctttggccgcattgccgatcgtcgcggtcgcaagaccagcatgctgattagcgtgtgcatgatgtgctttggctccctgattattgcctgcctccccggctatgatgccattggcacctgggcccccgccctgctcctgctggcccgcctctttcaaggcctgagcgtgggcggtgaatacggcaccagcgccacctatatgagtgaaattgccctggagggccgcaaaggtttttacgccagttttcaatatgtgaccctgattggcggtcaactgctcgccattctcgtggtggtgattctccaacaaattctgaccgattcccaactgcacgaatggggctggcgcattccctttgccatgggtgccgccctggccattgtggccctgtggctccgtcgccaactcgatgaaaccagccaaaaagaggtgcgcgccctgaaagaagccggcagttttaaaggtctctggcgcaaccgcaaggcctttctcatggtgctgggctttaccgccggcggtagtctgtccttttacacctttaccacctacatgcaaaaatatctcgtgaacaccaccggcatgcacgccaatgtggccagcgtgattatgaccgccgccctgtttgtgtttatgctcattcaacccctgattggcgccctcagcgataagattggtcgtcgcaccagtatgctgatttttggcggtatgagtgccctctgcaccgtgcccattctcaccgccctgcaacacgtgtccagcccctacgccgcctttgccctcgtgatgctggccatggtgattgtgtccttttataccagcattagtggcattctgaaggccgaaatgtttcccgcccaagtgcgcgccctgggcgtgggtctcagttacgccgtggccaatgccctgtttggcggttccgccgaatatgtggccctgtccctcaaaagctggggcagtgagaccacctttttctggtacgtgaccattatgggtgccctggcctttattgtgagcctgatgctccaccgcaaaggcaagggtattcgcctctagggtaccaggcaaacccatccccaaccccctgctgggcctggatagcaccggtggtggtcaccaccaccatcaccactagagtactgtatgcatcgagtgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctt

SEQ ID NO: 41 - DNA, 223 bp, Unknown
  Description of Unknown psbA promoter sequence
atttagcgtcttctaatccagtgtagacagtagttttggctccgttgagcactgtagccttgggcgatcgctctaaacattacataaattcacaaagttttcgttacataaaaatagtgtctacttagctaaaaattaagggttttttacacctttttgacagttaatctcctagcctaaaaagcaagagtttttaactaagactcttgccctttacaacctc

SEQ ID NO: 42 - DNA, 158 bp, Unknown
  Description of Unknown rrnB terminator sequence
tgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctt

SEQ ID NO: 43 - DNA, 1578 bp, Artificial Sequence
  Description of Artificial Sequence Synthetic polynucleotide
gaggttgtaaagggcaagagtcttagttaaaaactcttgctttttaggctaggagattaactgtcaaaaaggtgtaaaaaacccttaatttttagctaagtagacactatttttatgtaacgaaaactttgtgaatttatgtaatgtttagagcgatcgcccaaggctacagtgctcaacggagccaaaactactgtctacactggattagaagacgctaaatggtacctacgatctcatatgatacacgctccaagtagatggggagtatttccctcactagggttatgcagcccggacgttgtgtggaatgagcatccgagcctgtacatggacaaagaggaaaccagcatgaccaacctgcagacctttgaactgccgaccgaagtgaccggttgcgcggcggacatcagcctgggtcgtgcgctgattcaggcgtggcaaaaggatggtatcttccagattaaaaccgacagcgagcaggatcgtaagacccaagaagcgatggcggcgagcaagcaattttgcaaagagccgctgaccttcaaaagcagctgcgttagcgacctgacctacagcggttatgtggcgagcggcgaggaagttaccgcgggcaagccggatttcccggaaatttttaccgtgtgcaaggacctgagcgtgggcgatcagcgtgttaaagcgggttggccgtgccatggtccggttccgtggccgaacaacacctatcaaaagagcatgaaaacctttatggaggaactgggtctggcgggcgagcgtctgctgaaactgaccgcgctgggttttgaactgccgatcaacaccttcaccgacctgacccgtgatggctggcaccacatgcgtgtgctgcgtttcccgccgcagaccagcaccctgagccgtggtattggtgcgcacaccgactacggtctgctggtgattgcggcgcaagacgatgttggtggcctgtatatccgtccgccggtggagggcgaaaagcgtaaccgtaactggctgccgggcgagagcagcgcgggcatgtttgagcacgacgaaccgtggaccttcgttaccccgaccccgggtgtgtggaccgtttttccgggcgatattctgcagttcatgaccggtggccaactgctgagcaccccgcacaaggttaaactgaacacccgtgaacgtttcgcgtgcgcgtactttcacgagccgaacttcgaagcgagcgcgtatccgctgttcgagccgagcgcgaacgaacgtatccactacggcgagcacttcaccaacatgtttatgcgttgctatccggatcgtatcaccacccaacgtattaacaaagaaaaccgtctggcgcacctggaagacctgaagaaatacagcgacacccgtgcgaccggcagccaccaccaccatcatcattaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 44 - DNA, 48 bp, Unknown
  Description of Unknown T7 terminator sequence
caaaaaacccctcaagacccgtttagaggccccaaggggttatgctag

SEQ ID NO: 45 - DNA, 3943 bp, Artificial Sequence
  Description of Artificial Sequence Synthetic polynucleotide
gagcgtgtatcatatgagatctgacgctccttcgaggttgtaaagggcaagagtcttagttaaaaactcttgctttttaggctaggagattaactgtcaaaaaggtgtaaaaaacccttaatttttagctaagtagacactatttttatgtaacgaaaactttgtgaatttatgtaatgtttagagcgatcgcccaaggctacagtgctcaacggagccaaaactactgtctacactggattagaagacgctaaatggtacctacgatctcatatgatacacgctccaagtagatggggagtatttccctcactagggttatgcagcccggacgttgtgtggaatgagcatccgagcctgtacatggacaaagaggaaaccagcatgaccaacctgcagacctttgaactgccgaccgaagtgaccggttgcgcggcggacatcagcctgggtcgtgcgctgattcaggcgtggcaaaaggatggtatcttccagattaaaaccgacagcgagcaggatcgtaagacccaagaagcgatggcggcgagcaagcaattttgcaaagagccgctgaccttcaaaagcagctgcgttagcgacctgacctacagcggttatgtggcgagcggcgaggaagttaccgcgggcaagccggatttcccggaaatttttaccgtgtgcaaggacctgagcgtgggcgatcagcgtgttaaagcgggttggccgtgccatggtccggttccgtggccgaacaacacctatcaaaagagcatgaaaacctttatggaggaactgggtctggcgggcgagcgtctgctgaaactgaccgcgctgggttttgaactgccgatcaacaccttcaccgacctgacccgtgatggctggcaccacatgcgtgtgctgcgtttcccgccgcagaccagcaccctgagccgtggtattggtgcgcacaccgactacggtctgctggtgattgcggcgcaagacgatgttggtggcctgtatatccgtccgccggtggagggcgaaaagcgtaaccgtaactggctgccgggcgagagcagcgcgggcatgtttgagcacgacgaaccgtggaccttcgttaccccgaccccgggtgtgtggaccgtttttccgggcgatattctgcagttcatgaccggtggccaactgctgagcaccccgcacaaggttaaactgaacacccgtgaacgtttcgcgtgcgcgtactttcacgagccgaacttcgaagcgagcgcgtatccgctgttcgagccgagcgcgaacgaacgtatccactacggcgagcacttcaccaacatgtttatgcgttgctatccggatcgtatcaccacccaacgtattaacaaagaaaaccgtctggcgcacctggaagacctgaagaaatacagcgacacccgtgcgaccggcagccaccaccaccatcatcattaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcgctagcaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtaggtaccatttagcgtc

SEQ ID NO: 46 - protein, 6 aa, Artificial Sequence
  Description of Artificial Sequence Synthetic 6xHis tag
HHHHHH